(19) United States
(12) Patent Application Publication
(10) Pub. No.: US 2023/0044222 A1
(43) Pub. Date: Feb. 9, 2023

(54) FLUID CHARACTERISTIC INDICATOR

(71) Applicant: Paul J. Gilbert, Phoenix, AZ (US)

(72) Inventor: Paul J. Gilbert, Scottsdale, AZ (US)

(21) Appl. No.: 17/972,417

(22) Filed: Oct. 24, 2022

Related U.S. Application Data
(63) Continuation of application No. PCT/US2021/028465, filed on Apr. 21, 2021.
(60) Provisional application No. 63/013,990, filed on Apr. 22, 2020.

(51) Int. Cl. A61B 5/15; G01N 21/78

(52) U.S. Cl. CPC A61B 5/150961; G01N 21/78; A61B 5/150412; A61B 5/150755; A61B 5/150221; A61B 5/150099; A61B 5/150419; A61B 5/150824

(57) ABSTRACT

An apparatus comprises a diffuser, a connection tube section forming a tubular fluid passage, a first indicating element section that indicates a measurement related to a body fluid disposed between a distal end and the connection tube section, a second indicating element section that indicates that the first indicating element section is suitable for use, and a housing having a generally tubular body. The diffuser is at least partially disposed in the generally tubular body. A fluid chamber adapted to receive fluid is cooperatively defined by the housing and the diffuser. The first indicating element section is visibly disposed in a first section of the housing. The second indicating element section is disposed within the fluid chamber. A micro-needle collector having pipettes to draw the fluid from a source to the distal end is attachable to the distal end.

### CROSS REFERENCE TO RELATED APPLICATIONS

This application is related to co-pending U.S. Nonprovisional patent application Ser. No. 17/972,080 also titled “Fluid Characteristic Indicator” and filed on even date herewith, both of which are continuation applications of Patent Cooperation Treaty International Application Number PCT/US2021/028465 titled “Fluid Characteristic Indicator” and filed on Apr. 21, 2021, which claims the priority benefit of U.S. Provisional Patent Application No. 63/013,990, titled “Fluid Characteristic Indicator” and filed Apr. 22, 2020, the priority benefit of both of which is hereby claimed, and the entirety of both of which is hereby incorporated by reference.

### FIELD OF THE INVENTION

This application relates to apparatus and methods for measuring fluid characteristics, and more particularly to apparatus and methods for measuring and indicating a characteristic of a body fluid such as may be obtained from a medical or veterinary patient.

### BACKGROUND OF THE INVENTION

Although a practitioner may obtain a sample of a fluid from a medical or veterinary patient and order a variety of laboratory tests thereon, the processing of the sample in the clinic, its transportation to the laboratory, processing and analysis in the laboratory, and reporting of results all cause delay and expense. It may be desirable for the clinician to obtain an immediate and reliable indication of one or more characteristics of a fluid obtained from a patient.

For example, when inserting a nasogastric tube, the tube may be undesirably positioned in the patient's lungs, instead of the stomach. Introduction of fluid which was intended to be introduced into the patient's stomach into the lung instead may be disastrous. However, correct insertion of the nasogastric tube in the stomach may be confirmed by aspirating fluid through the tube and measuring the pH of the fluid. A pH below 6 is characteristic of stomach fluid and signals the clinician that the tube has been correctly inserted.

Although there are a variety of supplies and equipment that could, in theory, be used in a clinical setting to measure a fluid characteristic, it is not always convenient or safe to perform reliable and accurate measurements. Test equipment may not necessarily be available in the clinic at the time it is needed, may require calibration or preparation of the equipment or accessories before use, cleaning after use, or non-trivial training of personnel for correct measurement, operation, and interpretation of the result. Also, the cost of some equipment makes it unaffordable in some clinics. The test results may be needed immediately, which can make obtaining and interpreting an accurate measurement challenging. Also, the sample itself may be unstable, either generally, over time, or as a result of exposure to air, test probes, containers, reagents, materials, or the like, which allows only limited time for performing a measurement. Some test equipment or procedures require that the sample be handled in the open, which can result in spills and exposure of personnel to the sample, which may present chemical, biological, or radiological hazards.

### SUMMARY

There are disclosed herein several example embodiments of systems, apparatus, and methods for obtaining and containing a fluid obtained from a person or animal, measuring or detecting one or more characteristics of the fluid, displaying at least a first indication relating to the measurement, and displaying at least a second indication as to whether the first indication or indications are suitable for use. Also disclosed herein are methods of constructing and using such systems and apparatus. The person or animal may be, for example but without limitation, a medical, dental, or veterinary patient. The fluid may be, for example but without limitation, a patient's body fluid, such as gastric aspirate, gastric fluid, blood, filtered blood, plasma, amniotic fluid, cerebral spinal fluid, feces, urine, sputum, saliva, other fluids obtained from a patient's body, and the like. The indicators could also be used to measure characteristics of particulates, granules, powders, other solids, and the like, which can be transported in or with fluids, including gases, and the term fluid herein is intended to include fluids with entrained particulates, granules, powders, other solids, and the like.

In an example embodiment, an apparatus includes a diffuser, the diffuser having: a generally longitudinal shape with a proximal end opposite a distal end; a tip section at the distal end; a connection tube section forming a tubular fluid passage extending from the proximal end toward the distal end along at least a part of the distance between the proximal end and the distal end; a first indicating element section disposed between the tip section and the connection tube section, the first indicating element section indicating a measurement related to a body fluid; and a second indicating element section disposed in the connection tube section, the second indicating element section indicating that the first indicating element section is suitable for use.

Another embodiment takes the form of a method comprising preparing a skin surface for a sample, applying a micro-needle collector to the skin surface, capturing an image of an indicator, transmitting the image, and receiving test results, the test results indicative of the first indicating element section and the second indicating element section.

Yet another embodiment takes the form of a kit comprising: an indicator, a micro-needle collector attachable to the distal end of the housing of the indicator, the micro-needle collector having a plurality of pipettes to draw the fluid from a source to the distal end of the housing, and surface preparation components.

These and other aspects are disclosed in more detail in the following detailed description and the accompanying drawings.

### BRIEF DESCRIPTION OF THE DRAWINGS

FIG. 1A is an isometric view of a first example embodiment 100 of apparatus for obtaining and containing a fluid obtained from a person or animal, measuring or detecting one or more characteristics of the fluid, and displaying an indication relating to the measurement, constructed according to an aspect of the present invention;

FIG. 1B is an end view of the indicator 100 of FIG. 1A, looking toward the proximal end thereof;

FIG. 1C is a half-sectioned view of the indicator 100 of FIGS. 1A-1B;

FIG. 1D is a further isometric view of the indicator 100 of FIGS. 1A-1D, taken from a different angle;

FIG. 50 is a diagram showing schematically a first example embodiment of a system or assembly including a fluid characteristic indicator constructed according to an aspect of the invention, along with environmental components with which the indicator may be used, wherein the indicator comprises first and second indicating elements or media;

FIG. 51 is a diagram showing schematically a second example embodiment of a system or assembly including a fluid characteristic indicator constructed according to an aspect of the invention, along with environmental components with which the indicator may be used, wherein the indicator comprises first and second indicating elements or media;

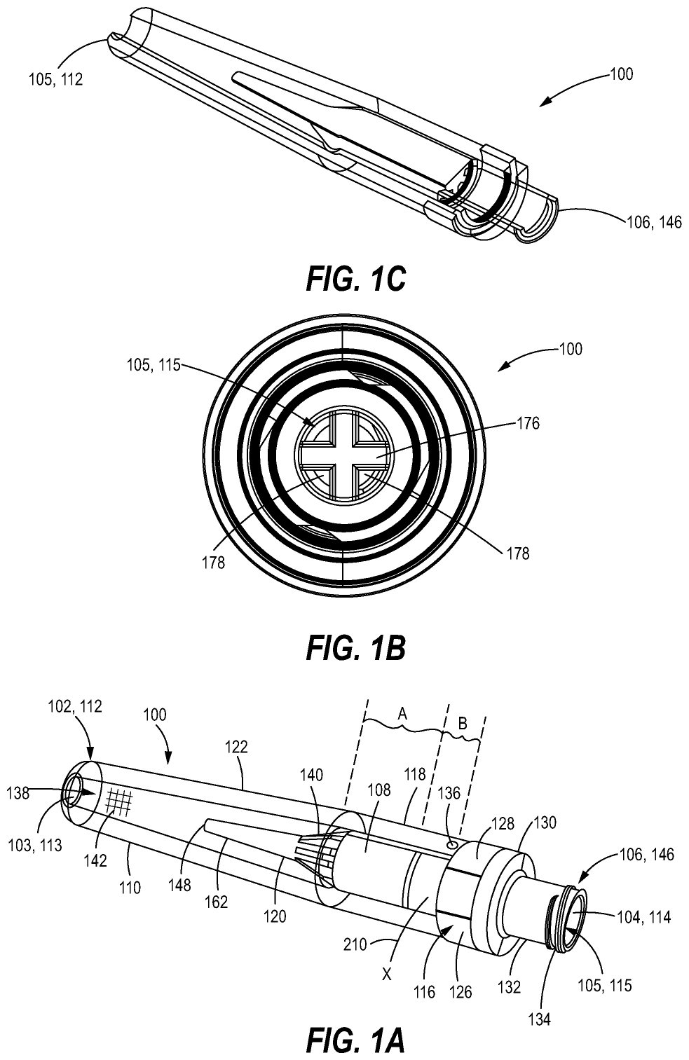

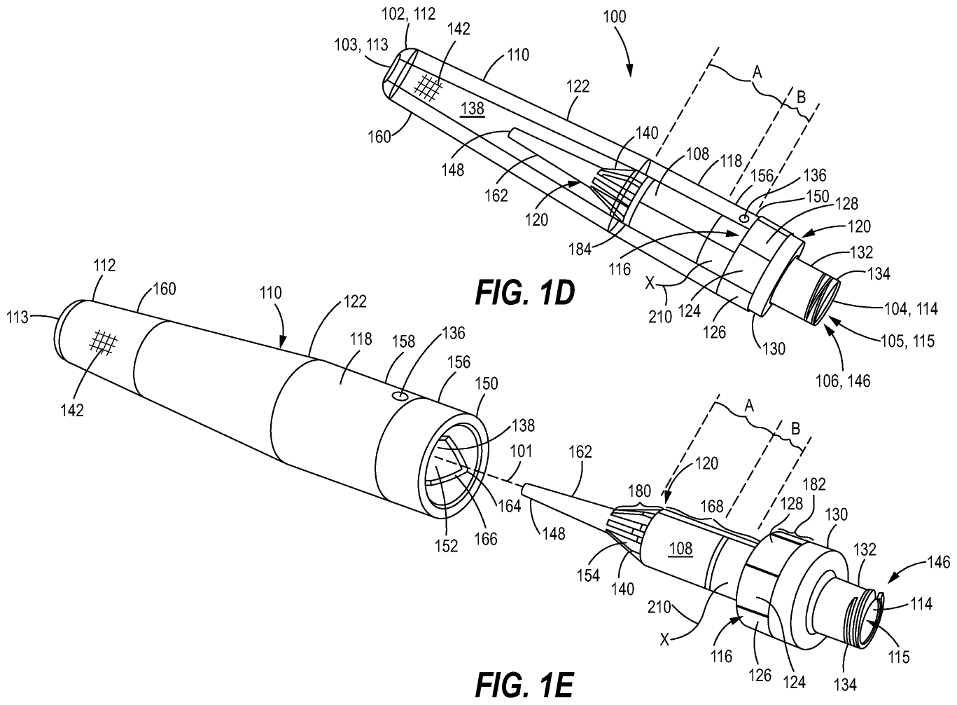

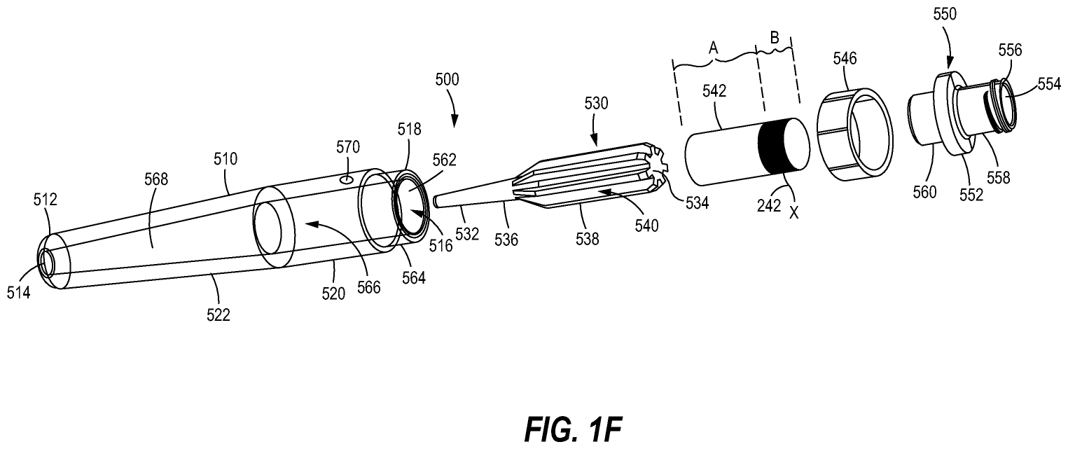

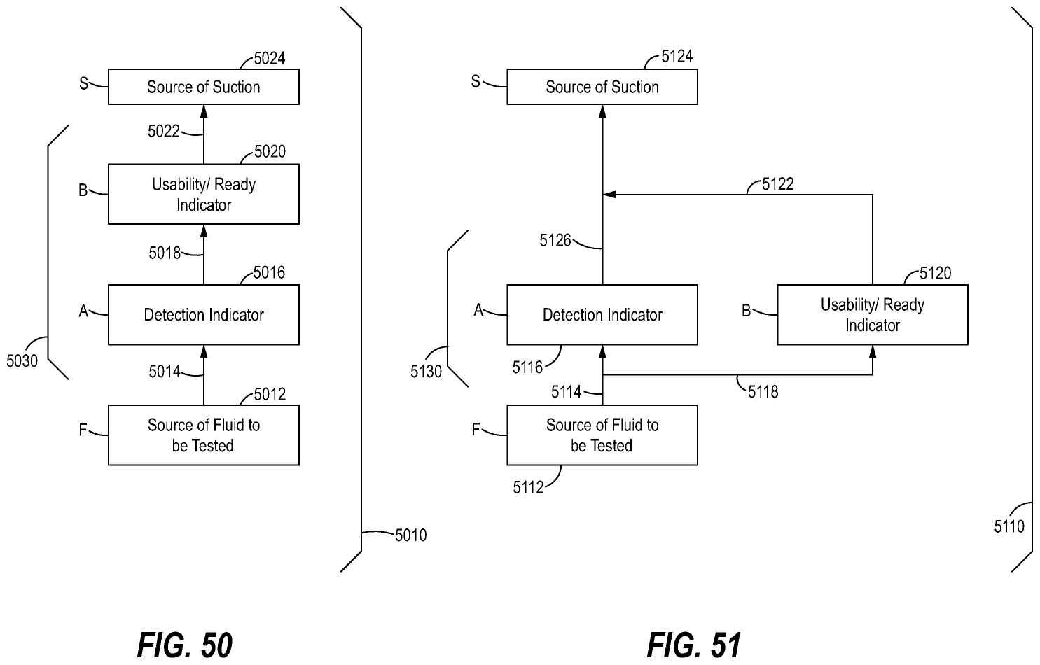

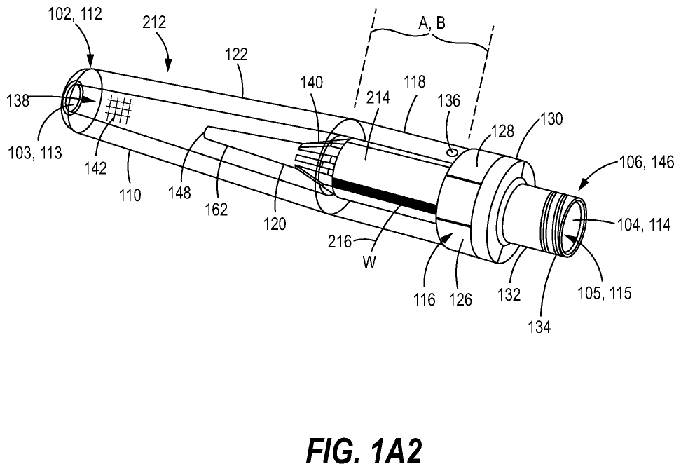

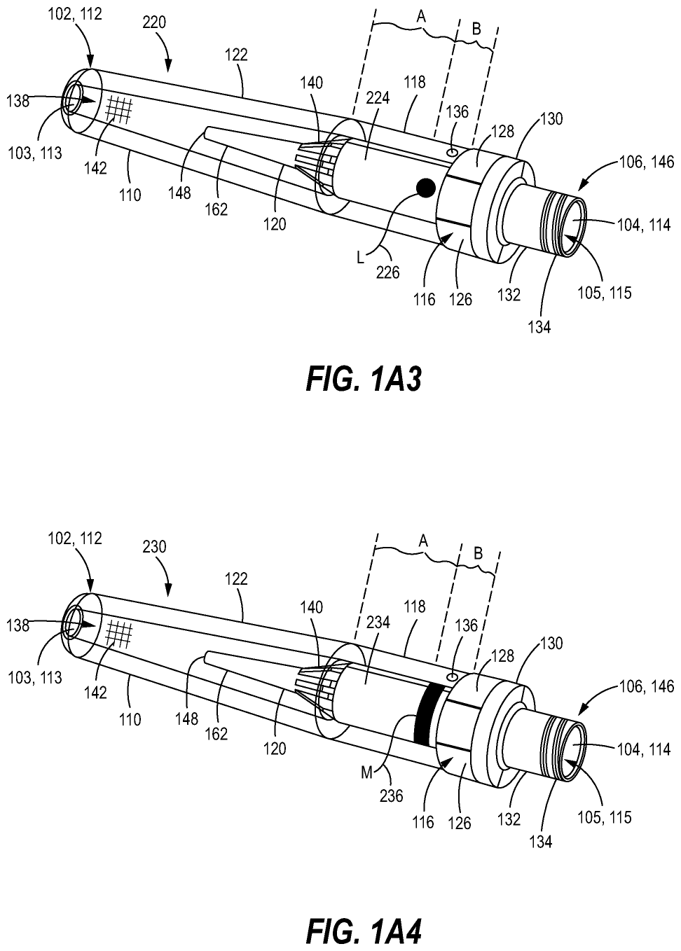

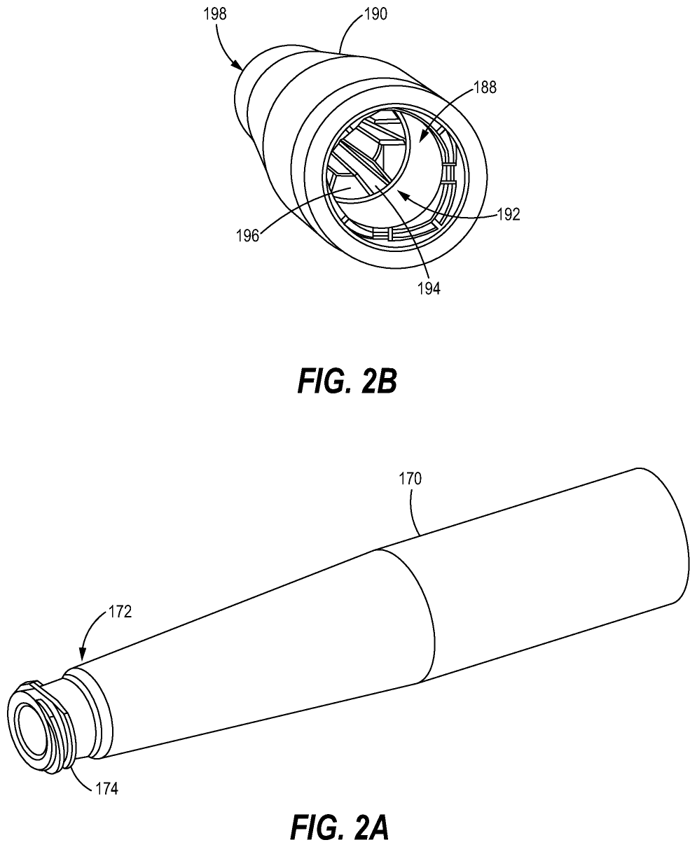

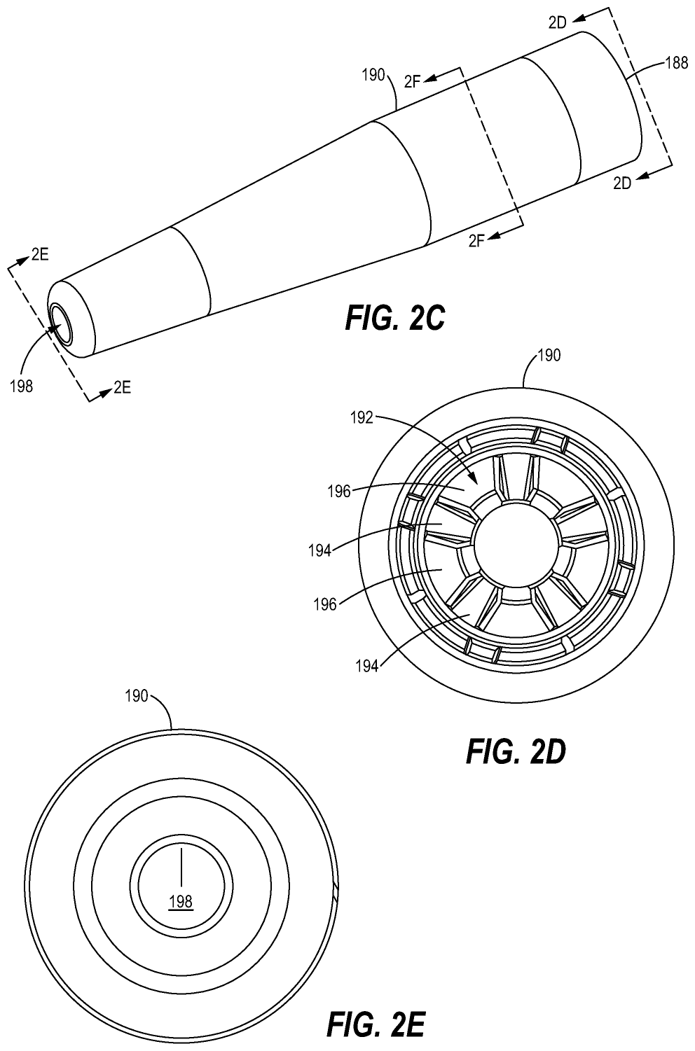

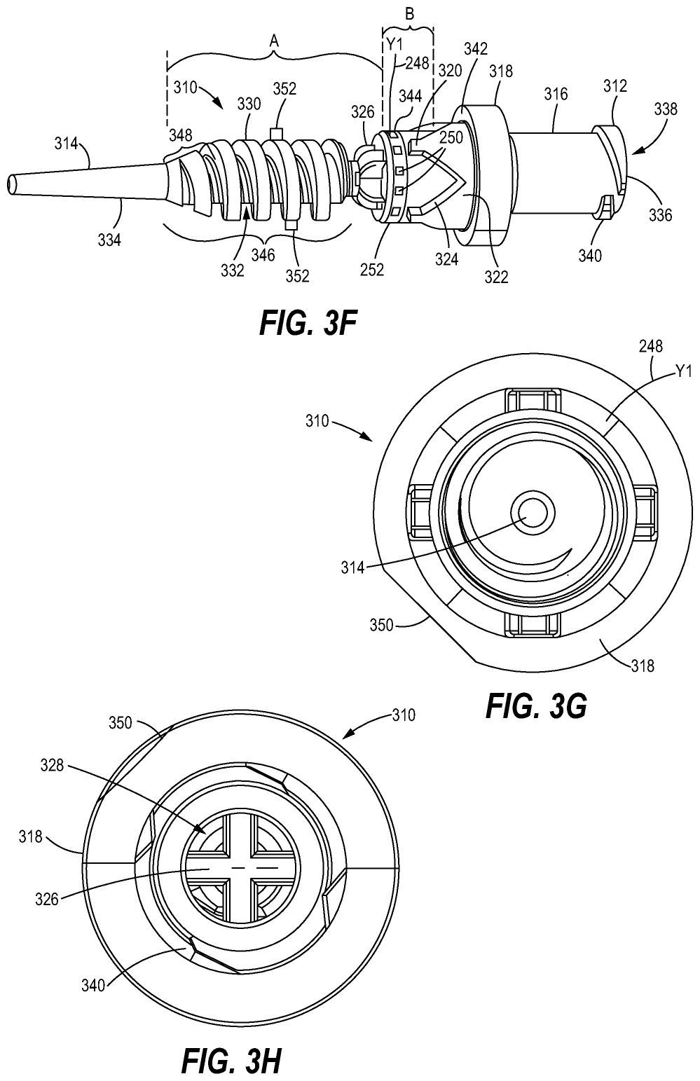

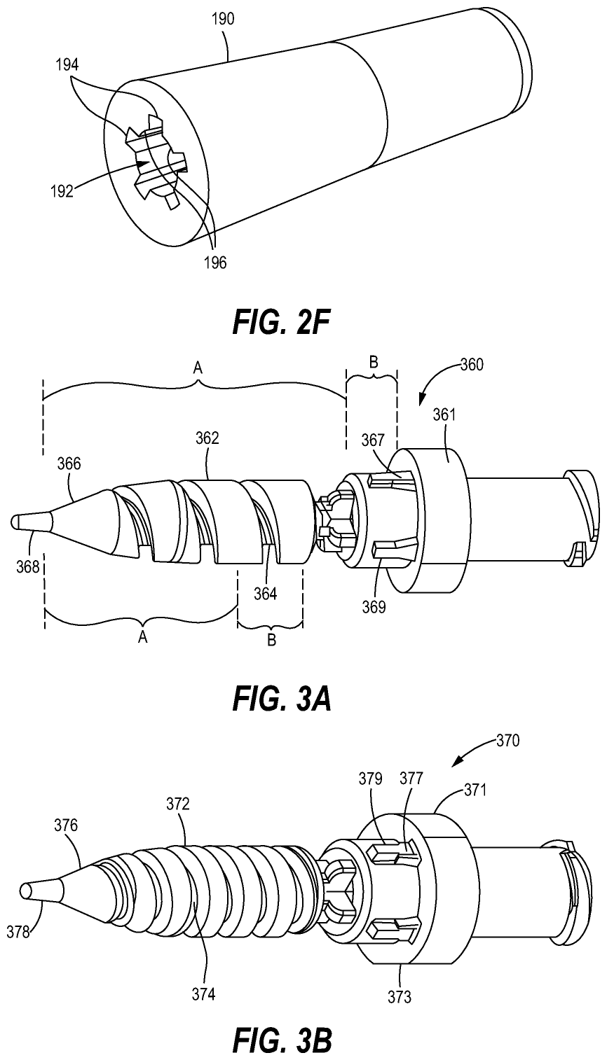

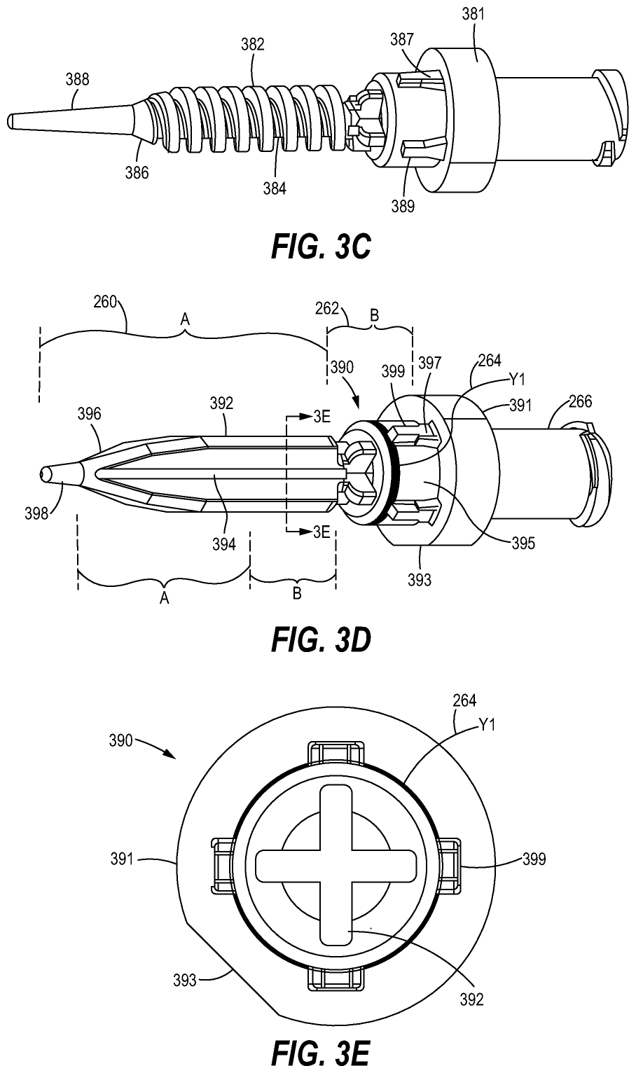

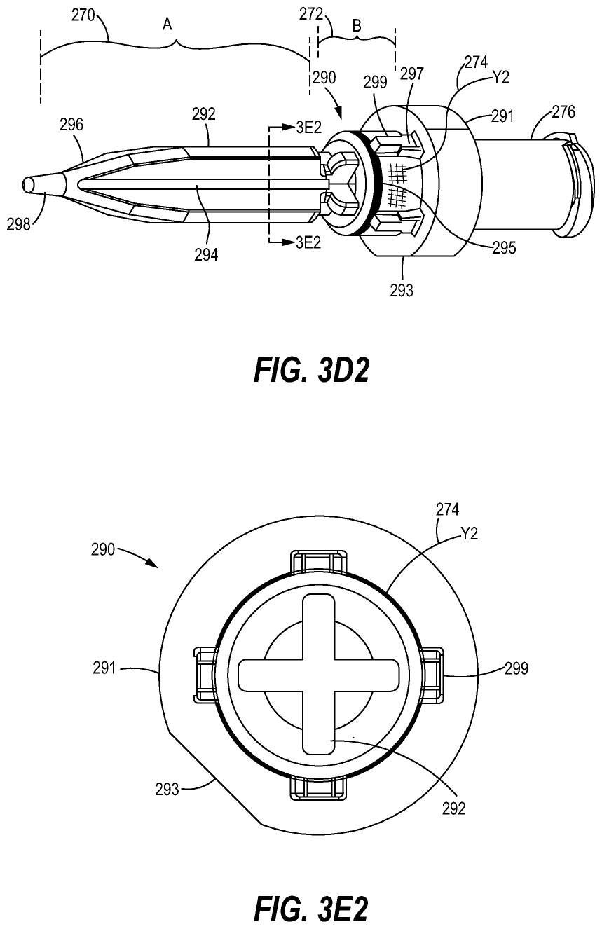

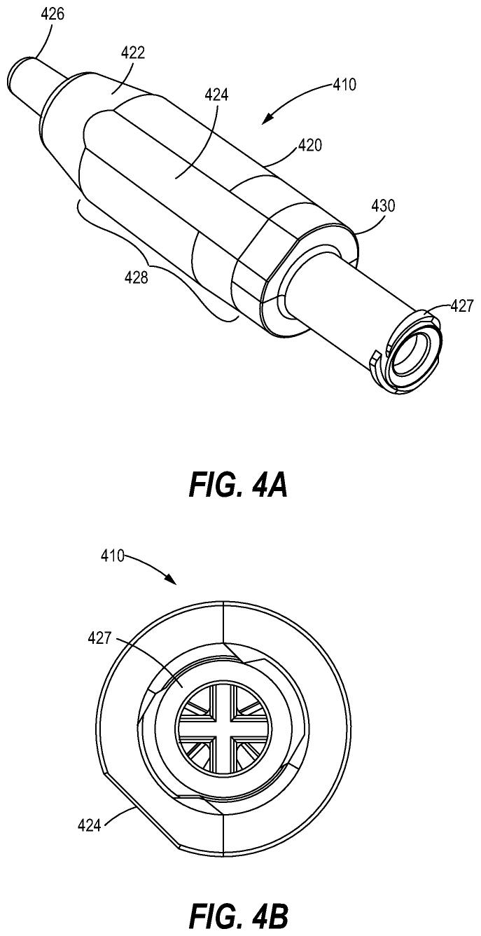

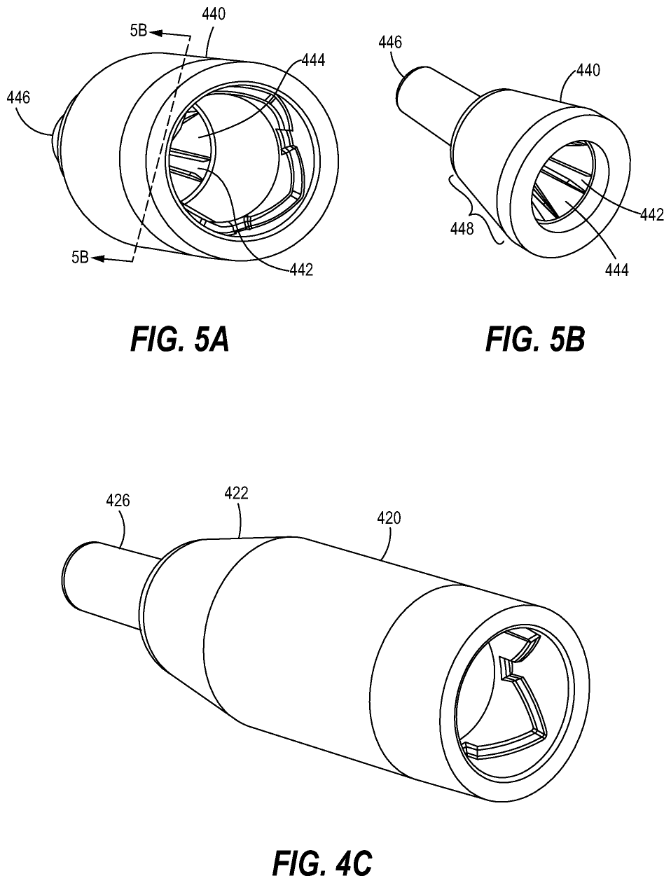

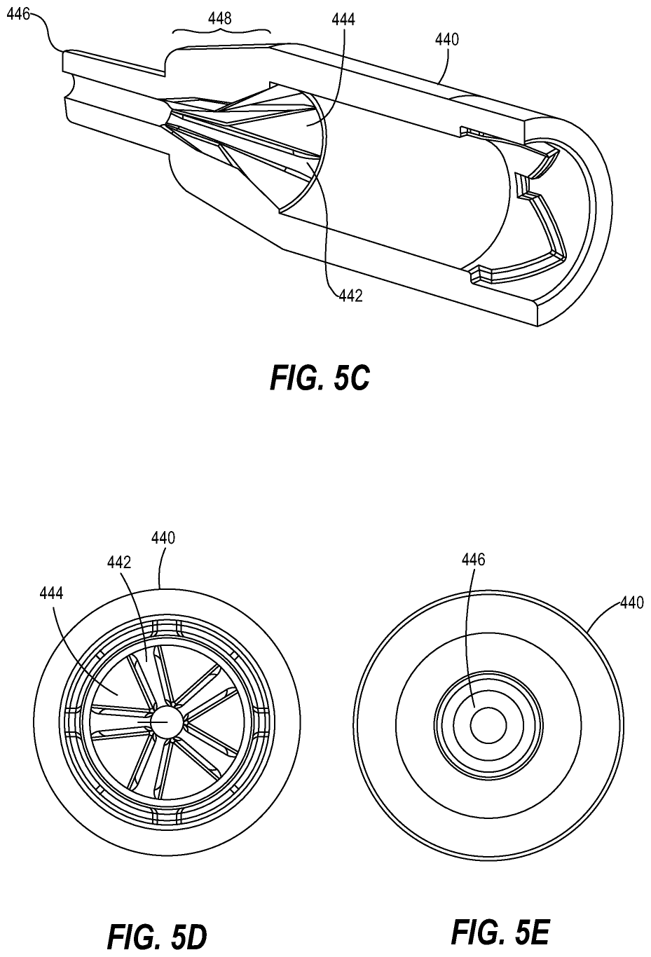

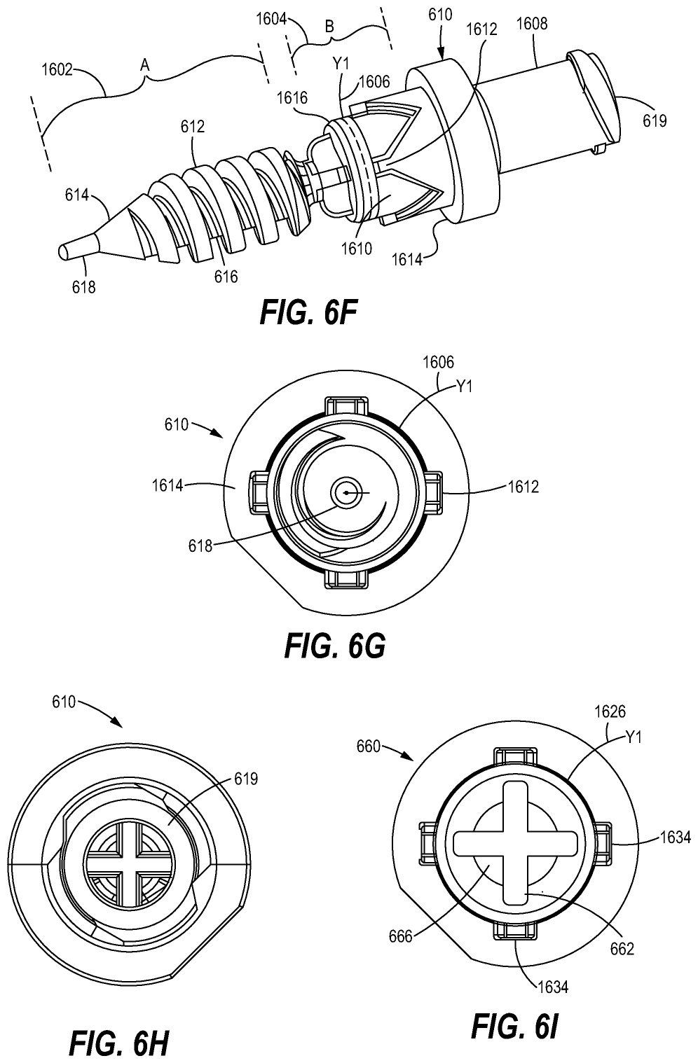

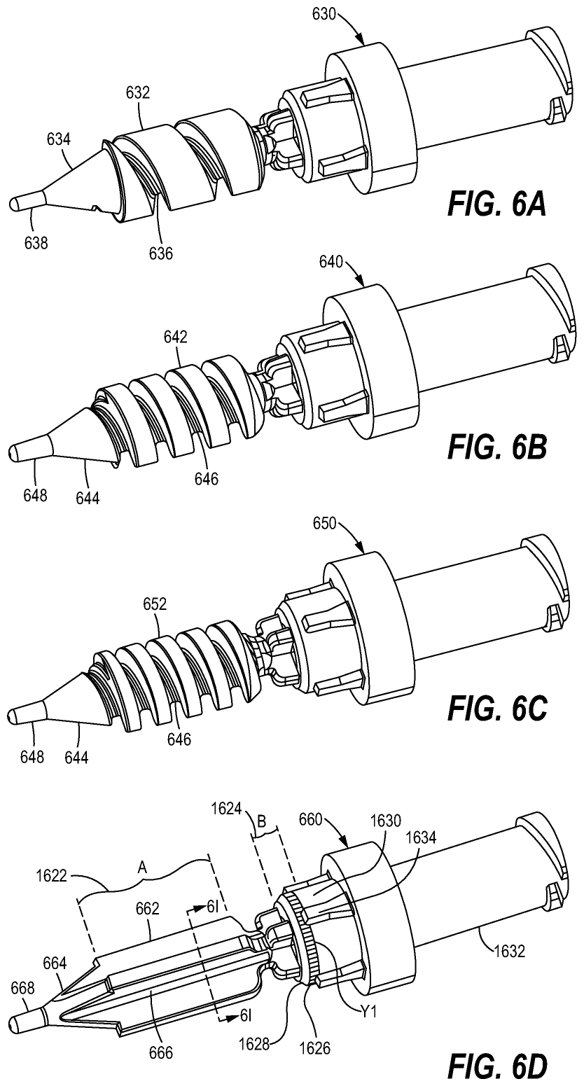

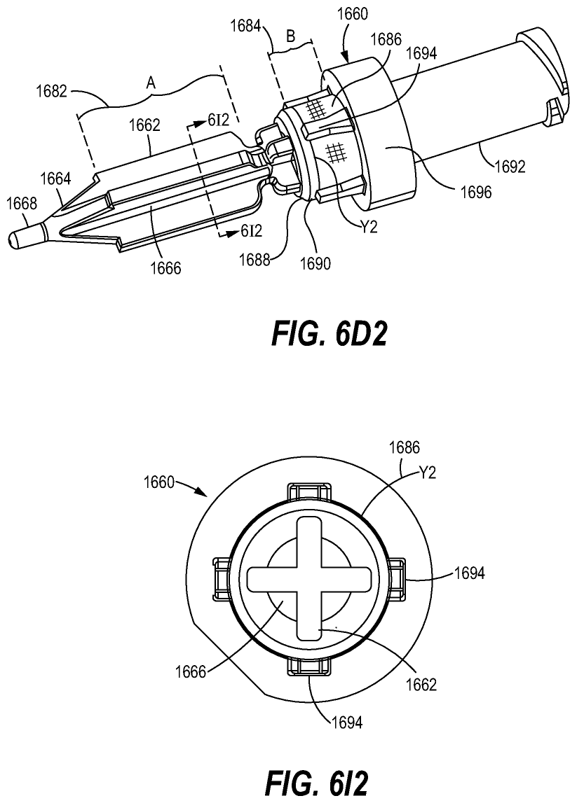

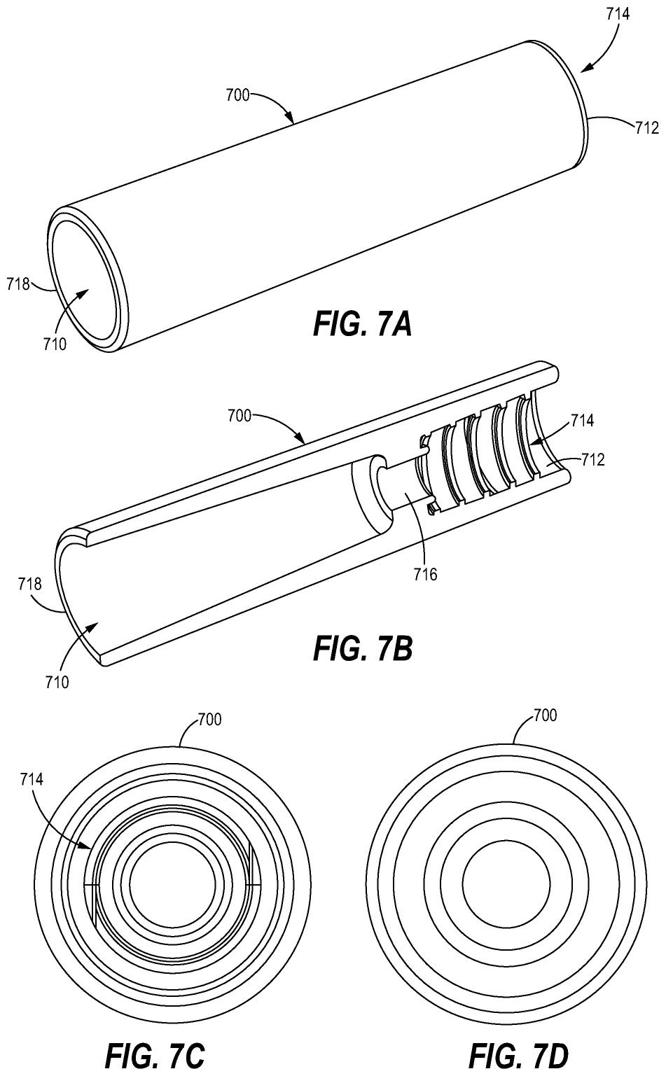

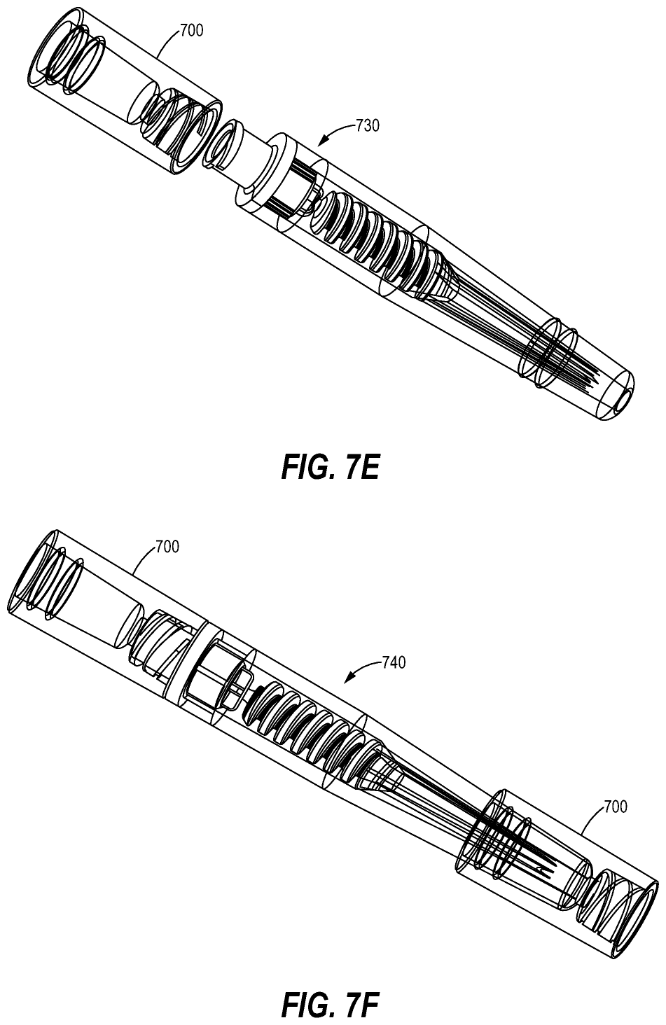

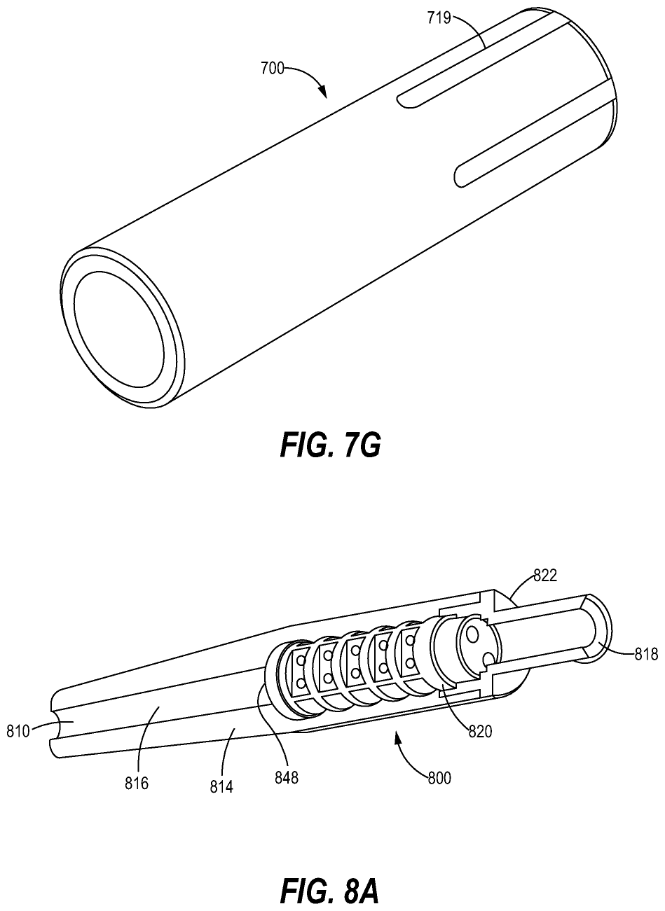

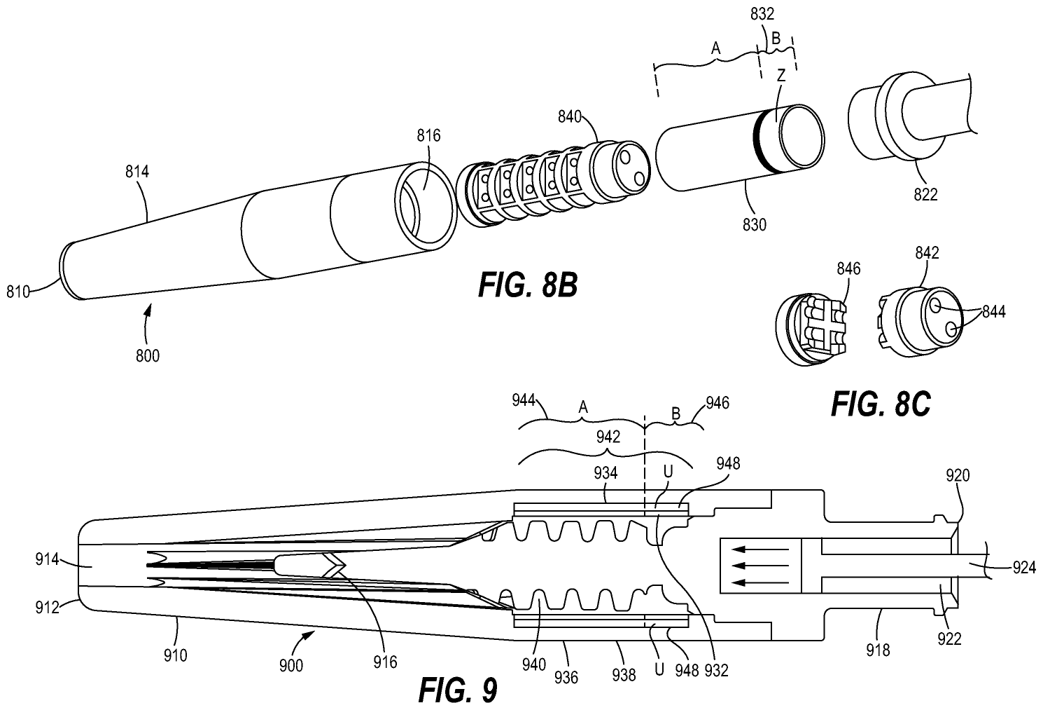

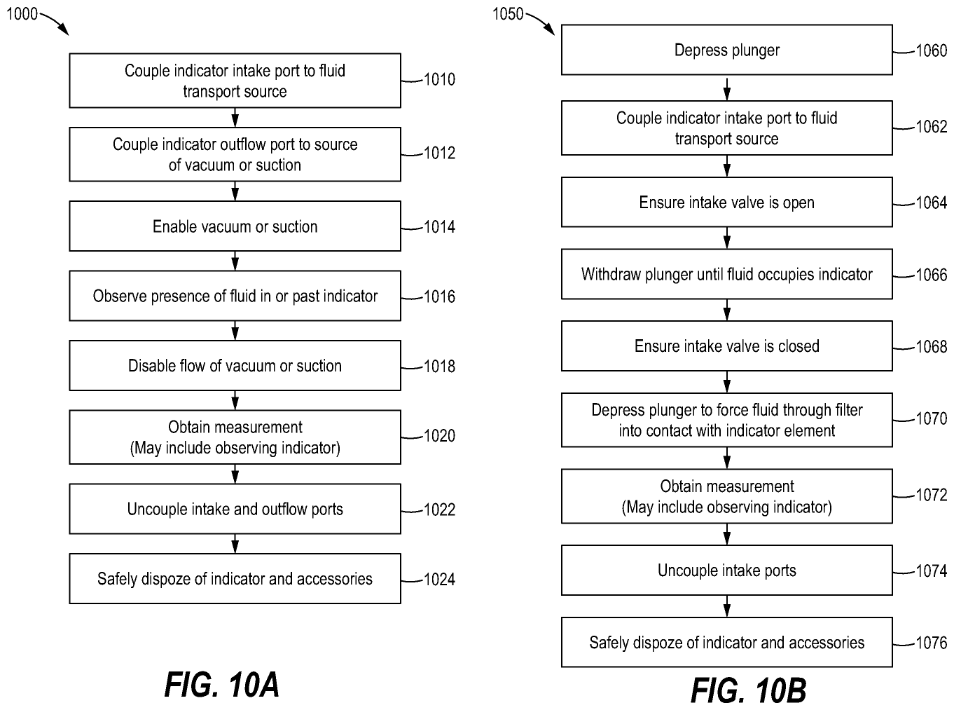

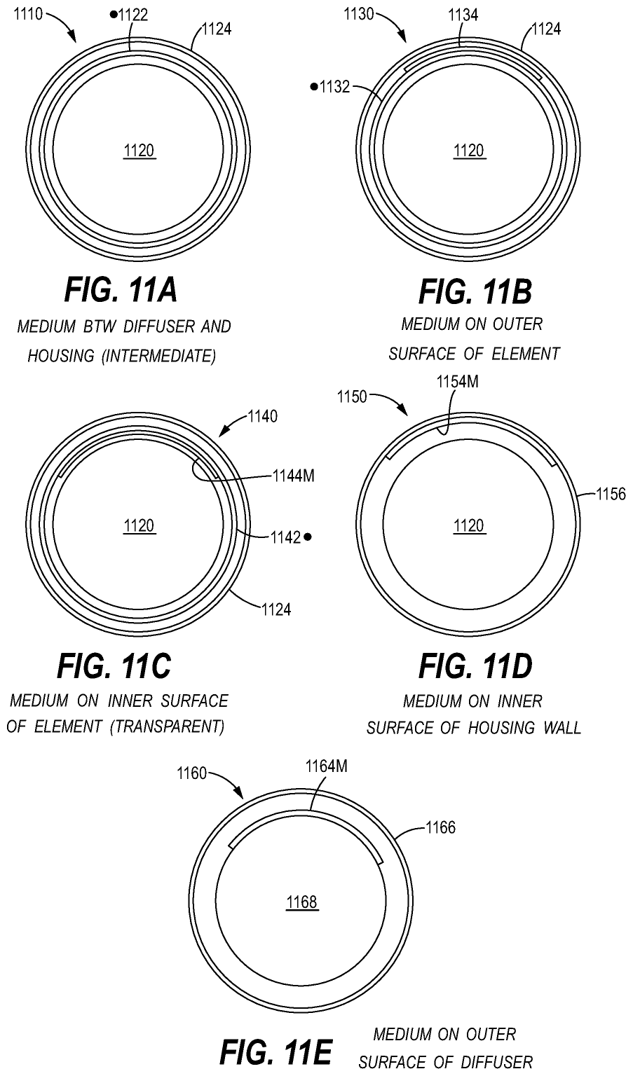

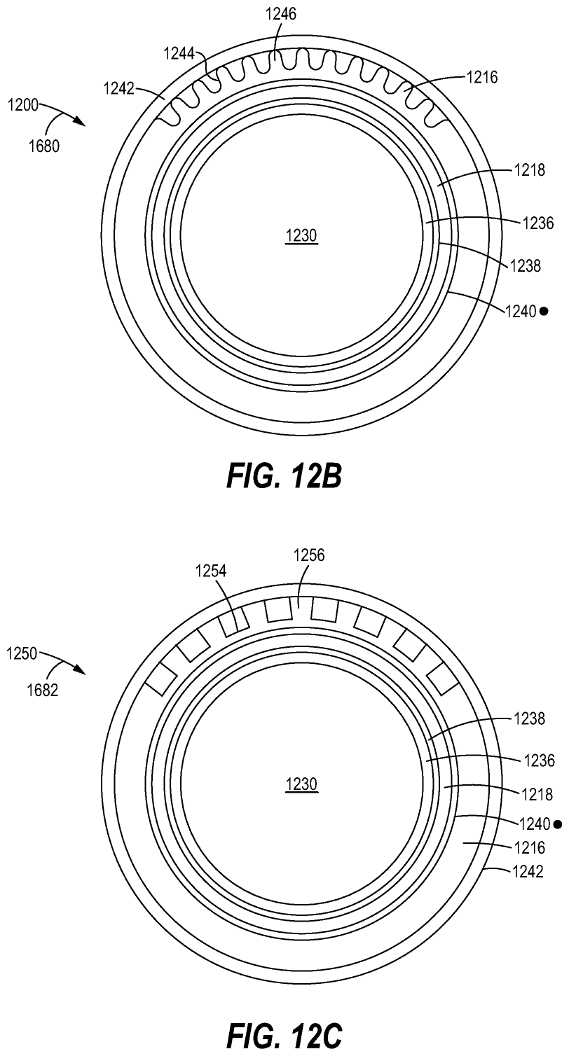

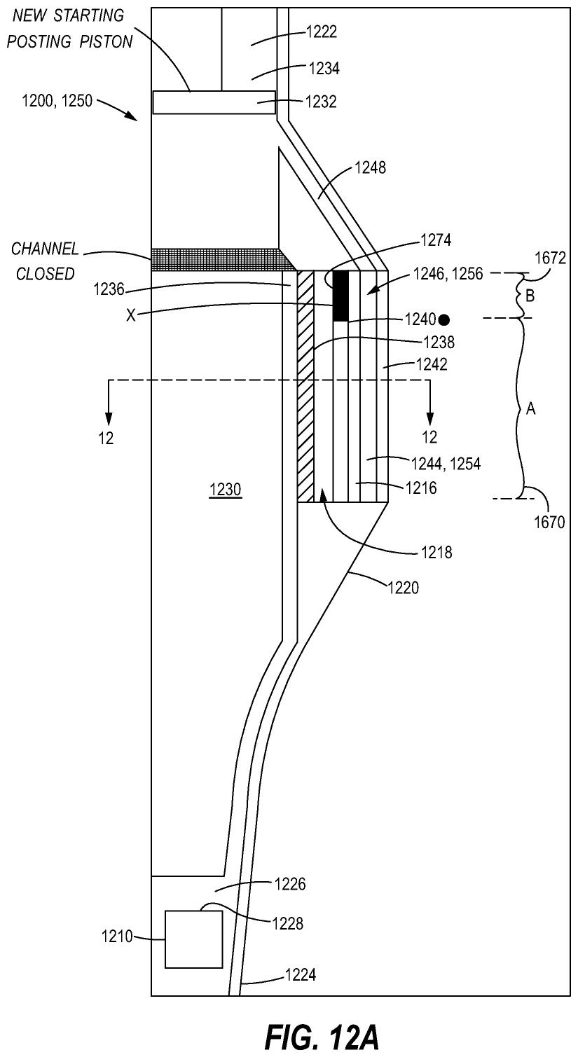

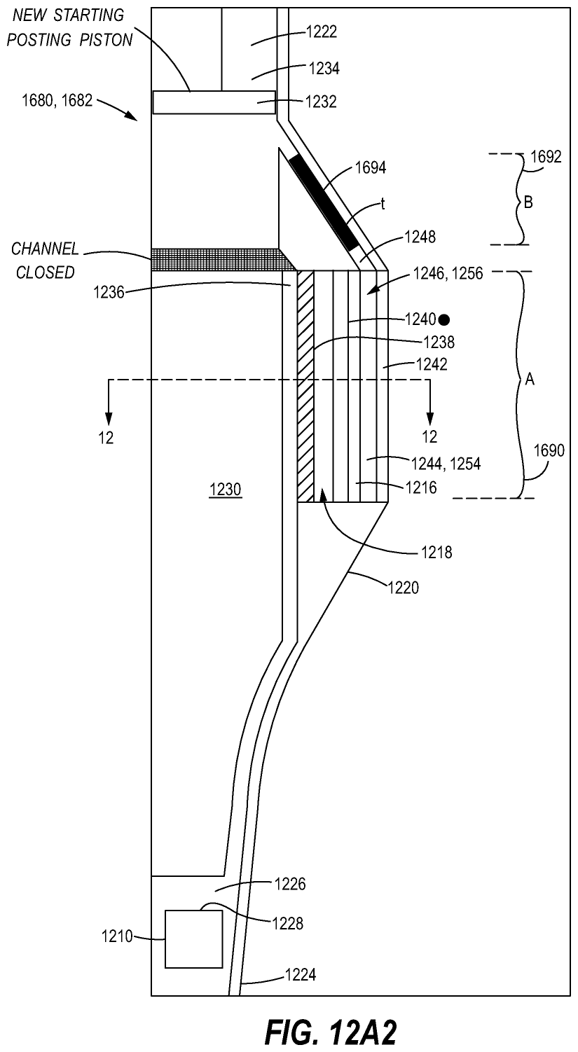

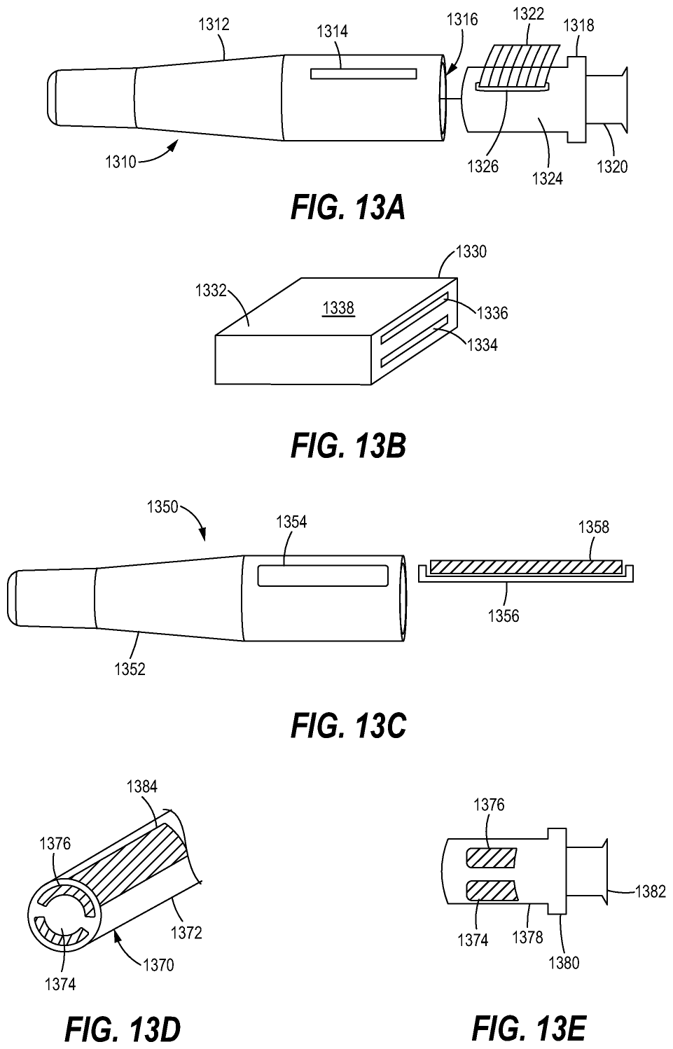

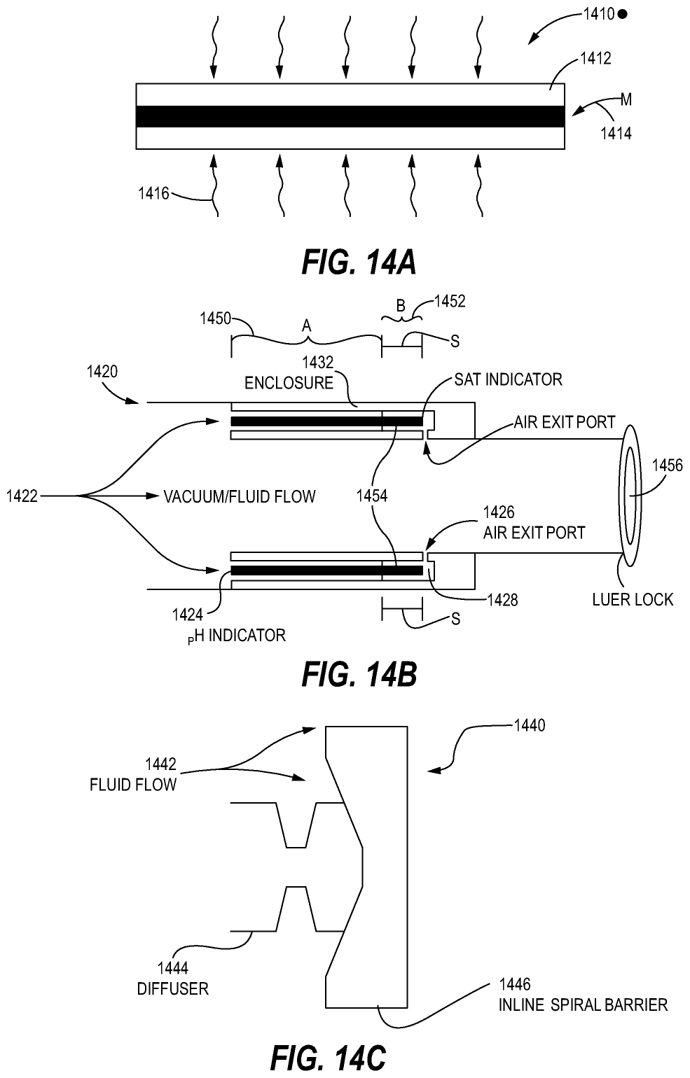

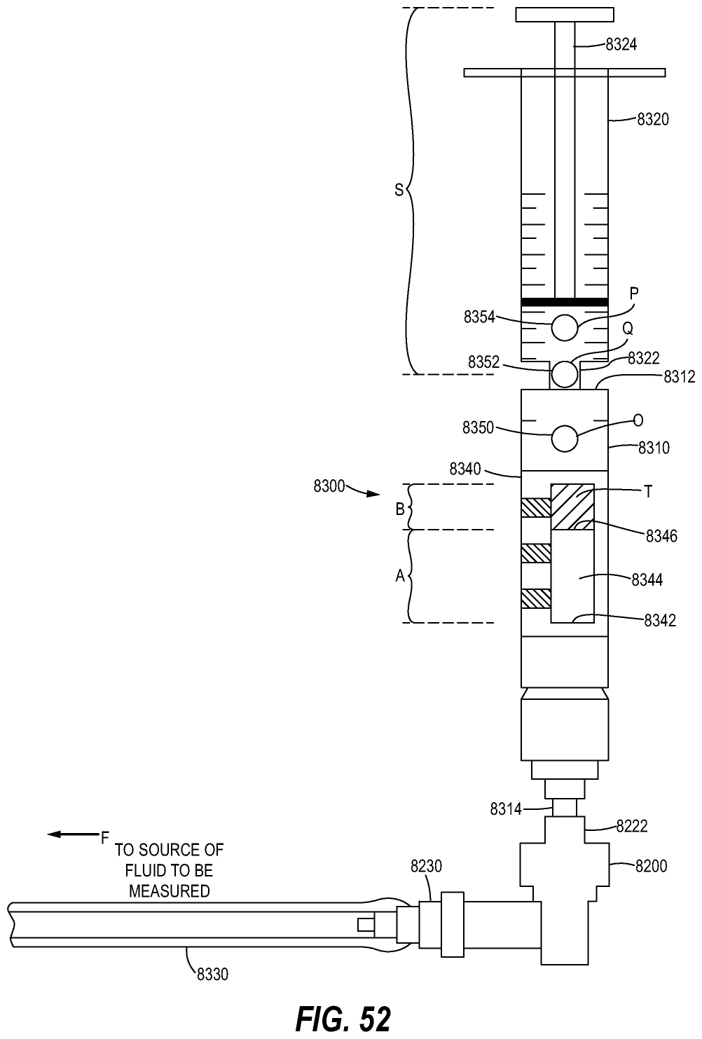

FIG. 1E is a partial exploded view depicting the housing 110 and diffuser 120 components of indicator 100 separately, for example, as they might appear prior to assembly as a unit;

FIG. 2A is a side view of a housing 170 of a further example embodiment of a body fluid characteristic indicator, according to a further aspect of the invention, the housing having a male Luer lock fitting at the distal end thereof;

FIG. 1A2 is an isometric view of a further example embodiment 212 of a fluid characteristic indicator including an example configuration of first and second indicating elements;

FIG. 1A3 is an isometric view of a further example embodiment 220 of a fluid characteristic indicator including an example configuration of first and second indicating elements;

FIG. 1A4 is an isometric view of a further example embodiment 230 of a fluid characteristic indicator including an example configuration of first and second indicating elements;

FIG. 2B is an isometric view of a further embodiment 190 of a housing according to an aspect of the present invention, which housing provides a central fluid passage of modified cross section;

FIG. 2C is a side view of the housing 190 of FIG. 2B;

FIG. 2D is a view of housing 190 of FIGS. 2B-2C looking into the proximal end opening 188 as shown by view lines 2D-2D of FIG. 2C;

FIG. 2E is a view of housing 190 of FIGS. 2B-2D looking into the distal end opening 198 as shown by view lines 2E-2E of FIG. 2C;

FIG. 2F is a cutaway view of the housing 190 of FIGS. 2B-2E, taken along the view lines 2F-2F of FIG. 2C.

FIG. 3F is a side isometric view of an alternative example embodiment of a diffuser 310 which may be used in constructing an indicator 100 in accord with a further aspect of the invention;

FIG. 3G is an end view of the diffuser 310 of FIG. 3F, taken from the distal end 314 thereof;

FIG. 3H is an end view of the diffuser 310 of FIGS. 3F and 3G, taken from the proximal end 312 thereof;

FIG. 3A is a side isometric view of a further alternative example embodiment 360 of a diffuser which may be used in constructing an indicator in accord with an aspect of the invention;

FIG. 3B is a side isometric view of a further alternative example embodiment 370 of a diffuser which may be used in constructing an indicator in accord with an aspect of the invention;

FIG. 3C is a side isometric view of a further alternative example embodiment 380 of a diffuser which may be used in constructing an indicator in accord with an aspect of the invention;

FIG. 3D is a side isometric view of a further alternative example embodiment 390 of a diffuser which may be used in constructing an indicator in accord with an aspect of the invention;

FIG. 3E is a partial cutaway view of the diffuser 390 of FIG. 3D taken along the view lines 3E-3E thereof;

FIG. 3D2 is a side isometric view of a further alternative example embodiment 290 of a diffuser which may be used in constructing an indicator in accord with an aspect of the invention;

FIG. 3E2 is a partial cutaway view of the diffuser 290 of FIG. 3D2 taken along the view lines 3E2-3E2 thereof;

FIG. 4A is an isometric view of a further example embodiment 410 of an indicator device constructed according to an aspect of the invention;

FIG. 4B is an end view of the indicator 410 of FIG. 4A, looking toward the proximal end 427 thereof;

FIG. 4C is a side isometric view of the indicator 420 of FIGS. 4A-4B;

FIG. 5A is a view of a further example embodiment 440, constructed according to an aspect of the present invention, of a housing which is similar to the housing 420 of FIGS. 4A and 5C, but which provides a central fluid passage modified cross section to incorporate fluid guide channels;

FIG. 5B is a cutaway view of the housing 440 of FIG. 5A taken along the view lines 5B-5B, looking into the housing from its proximal end;

FIG. 5C is a is a partial cutaway view of the housing 440 of FIGS. 5A-5B;

FIG. 5D is an end view looking into the housing 440 of FIGS. 5A-5C from the proximal end thereof;

FIG. 5E is an end view of the housing 440 of FIGS. 5A-5D looking toward the distal end 446 thereof;

FIG. 6F is an isometric view of a further example embodiment 610 of a diffuser, according to an aspect of the invention, exhibiting a lower-fluid-volume modification;

FIG. 6G is an end view of the diffuser 610 of FIG. 6F, looking toward the distal end 618 thereof;

FIG. 6H is an end view of the diffuser 610 of FIGS. 6F-6G, looking toward the proximal end 619 thereof;

FIG. 6A is a side view of a further example embodiment 630, according to an aspect of the invention, of a diffuser exhibiting a modification to accommodate lower fluid volume than earlier-described embodiments;

FIG. 6B is a side view of a further example embodiment 640, according to an aspect of the invention, of a diffuser exhibiting a modification to accommodate lower fluid volume than earlier-described embodiments;

FIG. 6C is a side view of a further example embodiment 650, according to an aspect of the invention, of a diffuser exhibiting a modification to accommodate lower fluid volume than earlier-described embodiments;

FIG. 6D is a side view of a further example embodiment 660, according to an aspect of the invention, of a diffuser exhibiting a modification to accommodate lower fluid volume than earlier-described embodiments;

FIG. 6I is an end view of the diffuser 660 of FIG. 6D, taken along the view lines 61-61 thereof, looking toward the distal end 668 thereof;

FIG. 6D2 is a side view of a further example embodiment 1660, according to an aspect of the invention, of a diffuser exhibiting a modification to accommodate lower fluid volume than earlier-described embodiments;

FIG. 6I2 is an end view of the diffuser 1660 of FIG. 6D2, taken along the view lines 612-612 thereof, looking toward the distal end 1668 thereof;

FIG. 1F is a partial exploded view of further example embodiment 500 of a body fluid characteristic indicator, according to a further aspect of the invention;

FIG. 7A is a side view of an adapter 700 constructed according to an aspect of the present invention for converting from a catheter tip connection to a locking or threaded fitting such as a Luer lock fitting;

FIG. 7B is a cutaway view of the adapter 700 of FIG. 7A;

FIG. 7C is an end view of the adapter 700 of FIGS. 7A-7B looking toward the proximal end 712 thereof;

FIG. 7D is an end view of the adapter 700 of FIGS. 7A-7C looking toward the distal end 718 thereof;

FIG. 7G is a side view of the adapter 700 showing striations 719 which may optionally be applied thereto;

FIG. 7E is a side stylized transparent view of the adapter 700 of FIGS. 7A-7D as it may be used with an example embodiment 730 of an indicator according to an aspect of the invention, prior to assembly of the adapter 700 on the indicator 730;

FIG. 7F is a side stylized transparent view of two instances of the adapter 700 of FIGS. 7A-7D as they may be assembled onto each end of an example embodiment 740 of an indicator according to an aspect of the invention;

FIG. 8A is a partial cutaway view of another example embodiment 800 of an indicator constructed according to a further aspect of the invention;

FIG. 8B is an exploded view of the indicator 800 of FIG. 8A;

FIG. 8C is an exploded view showing two instances of blocks 842 which may be used in the indicator 800 of FIGS. 8A and 8B;

FIG. 9 is a cross-section view of another example embodiment 900 of an indicator constructed according to a further aspect of the invention, in which the indicator provides a filter to filter the fluid to which the indicator element is exposed;

FIG. 10A is a flow chart of an example embodiment of a method 1000, according to an aspect of the invention, which may be used in conjunction with embodiments of indicators of the types disclosed herein for obtaining a measurement or detection;

FIG. 10B is a flow chart of a further example embodiment of a method 1050, according to an aspect of the invention, which may be used in conjunction with embodiments of indicators of the types disclosed herein for obtaining a measurement or detection;

FIG. 11A is a simplified cross-section diagram of an example embodiment 1110 according to an aspect of the invention showing an arrangement of an indicating element 1122 formed as a tube or rolled section of a material having the measuring capability, and certain other components thereof;

FIG. 11B is a simplified cross-section diagram of an example embodiment 1130 according to an aspect of the invention showing an arrangement of an indicating element 1132, wherein the measuring component or medium is disposed on an outer surface of a tubular substrate, and certain other components thereof;

FIG. 11C is a simplified cross-section diagram of an example embodiment 1140 according to an aspect of the invention showing an arrangement of an indicating element 1142, wherein the measuring component or medium is disposed on an inner surface of a tubular substrate, and certain other components thereof;

FIG. 11D is a simplified cross-section diagram of an example embodiment 1150 according to an aspect of the invention showing an arrangement wherein the measuring component or medium is disposed on the inner surface of the wall 1156 of the housing;

FIG. 11E is a simplified cross-section diagram of an example embodiment 1160 according to an aspect of the invention showing an arrangement wherein the measuring component or medium is disposed on the outer surface of the diffuser 1168;

FIG. 12A depicts a simplified schematic cross-section view of further example embodiments 1200, 1250, of an indicator device constructed according to aspects of the invention;

FIG. 12B is a simplified cross-section view of indicator 1200 of FIG. 12A, taken along section line 12-12 of FIG. 12A;

FIG. 12C is a simplified cross-section view of indicator 1250 of FIG. 12A taken along section line 12-12 of FIG. 12A;

FIG. 12A2 depicts a simplified schematic cross-section view of further example embodiments 1680 and 1682, of an indicator device constructed according to aspects of the invention;

FIG. 13A depicts a partly-exploded side view of a further example embodiment 1310 of an indicator constructed according to aspects of the invention;

FIG. 13B is a side view of an example embodiment 1330 of a cassette constructed according to an aspect of the invention for storing a supply of indicating element sheets;

FIG. 13C is a partly-exploded side view of a further embodiment 1350 of an indicator constructed in accord with a further aspect of the invention;

FIG. 13D is a simplified schematic diagram of a further embodiment 1370 of an indicator constructed according to a further aspect of the invention, wherein there is provided a plurality of indicating elements;

FIG. 13E is a simplified schematic diagram of a further embodiment 1380 of a diffuser constructed according to a further aspect of the invention, and adapted, for example, for use with the indicator 1370 and housing 1372 of FIG. 13D;

FIG. 14A is a side cross-section view of a further embodiment 1410 of an indicating element constructed according to an aspect of the invention;

FIG. 14B is a simplified cross-section view of a further example embodiment of an indicator 1420 constructed according to the invention; and

FIG. 14C is a simplified cross section view of a further example embodiment 1440 of an indicator constructed according to the invention.

FIG. 52 is a side elevation view of a combination of apparatus 8300 comprising a fluid characteristic indicator and additional components for use in obtaining measurements of characteristics of fluid;

FIG. 53 is a set of three schematic depictions of an indicator, constructed according to an aspect of the invention, wherein the indicator includes a movable cover to aid a user in interpreting information displayed by a second indicating element thereof;

FIG. 54 is a detail view of a further example embodiment of a diffuser 1760 constructed according to an aspect of the invention;

FIG. 55 is a further detail view of the diffuser 1760 of FIG. 54, in which a second indicating element thereof is omitted so that additional structures and features may be seen;

FIG. 56 is a detail view of a further example embodiment of a diffuser 1800 constructed according to an aspect of the invention;

FIG. 57 is a further detail view of the diffuser 1800 of FIG. 56, in which a second indicating element thereof is omitted so that additional structures and features may be seen;

FIG. 58 is a further detail view of the diffuser 1800 of FIG. 56, in which a second indicating element thereof is omitted so that additional structures and features may be seen;

FIG. 59 is a detail view of a further example embodiment of a diffuser 1840 constructed according to an aspect of the invention;

FIG. 60 is a further detail view of the diffuser 1840 of FIG. 59, in which a second indicating element thereof is omitted so that additional structures and features may be seen;

FIG. 61 depicts a side-view of an indicator with a micro-needle collector obtaining a fluid sample from a fluid source, in accordance with an embodiment of the present disclosure;

FIG. 62 depicts a partially exploded view of the micro-needle collector, in accordance with an embodiment of the present disclosure;

FIG. 63 depicts an end view of the micro-needle collector, in accordance with an embodiment of the present disclosure;

FIG. 64 depicts a block diagram, in accordance with an embodiment of the present disclosure;

FIG. 65 depicts a method, in accordance with an embodiment of the present disclosure;

FIG. 66 depicts a perspective view of an indicator attached to a micro-needle collector 6100 obtaining a fluid from a patient arm;

FIG. 67 depicts the image of the indicator after a fluid has been collected by the indicator;

FIG. 68 depicts an indicator kit, in accordance with an embodiment of the present disclosure;

FIG. 69 depicts an indicator in a bulb-syringe configuration, in accordance with an embodiment of the present disclosure;

FIG. 70 depicts a needle and syringe configuration, in accordance with an embodiment of the present disclosure;

FIG. 71 depicts a first test-tube configuration of an indicator, in accordance with an embodiment of the present disclosure;

FIG. 72 depicts a vacuum container configuration, in accordance with an embodiment of the present disclosure;

FIG. 73 depicts a molded and encapsulated indicator, in accordance with an embodiment of the present disclosure; and

FIG. 74 depicts another embodiment of an indicator, in accordance with an embodiment of the present disclosure.

### DETAILED DESCRIPTION

There are disclosed herein several example embodiments of systems, apparatus, and methods for obtaining and containing a fluid obtained from a person or animal, measuring or detecting one or more characteristics of the fluid, and displaying an indication relating to the measurement. Also disclosed herein are methods of constructing and using such systems and apparatus. The person or animal may be, for example but without limitation, a medical, dental, or veterinary patient. The fluid may be, for example but without limitation, a patient's body fluid, such as gastric aspirate, gastric fluid, blood, filtered blood, plasma, amniotic fluid, cerebral spinal fluid, feces, sputum, urine, saliva, other fluids obtained from a patient's body, and the like. In some embodiments, these fluids may be mixed as with a gastrointestinal bleed. The term fluid is intended to include, without limitation, liquids, gases, mucous, gels, and may also include other matter amenable to handling in nasogastric tubes, feeding tubes, suction tubes, or similar tubing found in hospitals, physician, veterinarian, and dental offices, and other clinical settings. The indicators could also be used to measure characteristics of particulates, granules, powders, other solids, and the like, which can be transported in or with fluids, including gases, and the term fluid herein is intended to include fluids with entrained particulates, granules, powders, other solids, and the like.

Examples of tests that may be performed on the fluids include pH tests and immunoglobulin assays that would allow the determination of the presence or absence of certain proteins, including DNA fragments. One example of an immunoglobulin assay would be a plasma test of HCG (Human Chorionic Gonadotropin) test to determine pregnancy. The tests may also determine the presence of certain toxins, drugs, detection of dangerous levels of drugs or proteins—which may be performed via an immunoglobulin test on urine, and the like. Determining the presence of DNA fragments found in viruses or bacteria via immunoglobulin assay would be another potential use of the indicator.

FIG. 1A is an isometric view of a first example embodiment 100 of apparatus for obtaining and containing a fluid obtained from a person or animal, measuring or detecting one or more characteristics of the fluid, displaying at least a first indication relating to the measurement, and displaying at least a second indication as to whether the first indication or indications are suitable for use. The first example embodiment 100 is constructed according to an aspect of the present invention, and the apparatus may be referred to hereinafter as indicator device 100 or indicator 100. FIG. 1B is an end view of the indicator 100 taken from the proximal end 106 thereof. FIG. 1C is a half-section view of the indicator 100. FIG. 1D is another isometric view of indicator 100 taken from a different angle.

As best seen in FIGS. 1A-1D, indicator 100 preferably has a generally tubular shape having a distal end 102 and a proximal end 106. The indicator 100 has a distal-end or intake port 103 at the distal end 102, and a proximal-end or outflow port 104 at the proximal end 106. A fluid communication chamber, lumen, or passage 105 extends between the ports 103 and 104 and permits flow of fluid therethrough. The fluid passage 105 may include various volumes, spaces, conduits, channels, chambers, and the like in fluid communication within the indicator 100.

A first indicating element or medium 108 (or a first segment of such an element or medium, or a first group of such elements or media—labeled as the region ‘A’ throughout the figures) sensitive to a fluid characteristic or property to be measured or sensed is disposed within fluid passage 105 and is arranged such that fluid present therein will contact indicating element 108. A second indicating element or medium 210 (or a first segment of such an element or medium, or a first group of such elements or media—labeled as the region ‘B’ throughout the figures) sensitive to a fluid characteristic or property is disposed within fluid passage 105 and is also arranged such that fluid present therein will contact indicating element 210.

An indicator window 118, which is preferably substantially transparent, is provided in the indicator 100 to render the indicating elements 108 and 210 externally visible or observable. In general operation, the distal end port 103 of the indicator is coupled for fluid communication with a source, conduit, or reservoir of or containing a patient's body fluid or other fluid to be tested, which source may, for example, be a nasogastric tube or feeding tube inserted into the patient's stomach, or a suction tube in use in treating or diagnosing the patient. In some embodiments, the proximal end port 104 is coupled to a source of vacuum or suction so as to draw or aspirate fluid through distal end port 103 into fluid passage 105 and into contact with or in the vicinity of the indicating elements 108 and 210. Responsive to the contact with or the presence of the fluid, the indicating elements 108 and 201 may exhibit an externally observable indication, or change therein, relating to respective fluid characteristics or properties to be sensed or measured. For example but without limitation, the indicating elements 108 and 210 may be constructed of a pH-indicating paper which presents a color or shade or other visible indicia corresponding to the pH of the fluid, or a range thereof, or the value of the characteristic on one side or the other of a defined threshold. Indicating elements or media of other types, or sensitive to other fluid characteristics or properties, could also be used, as discussed further.

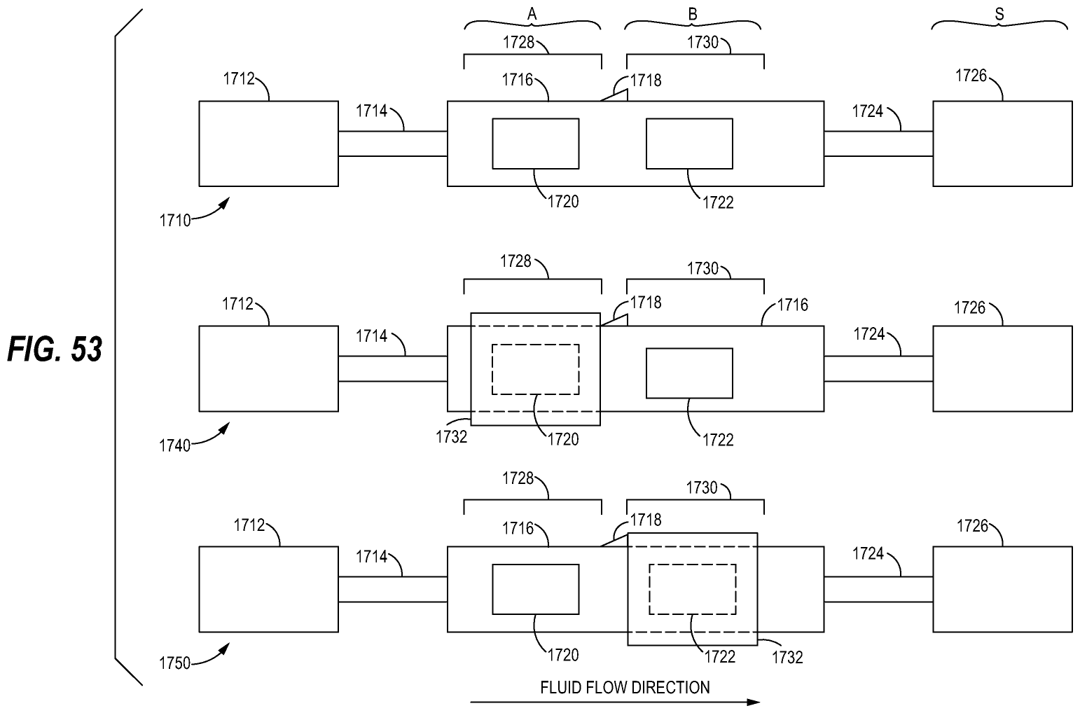

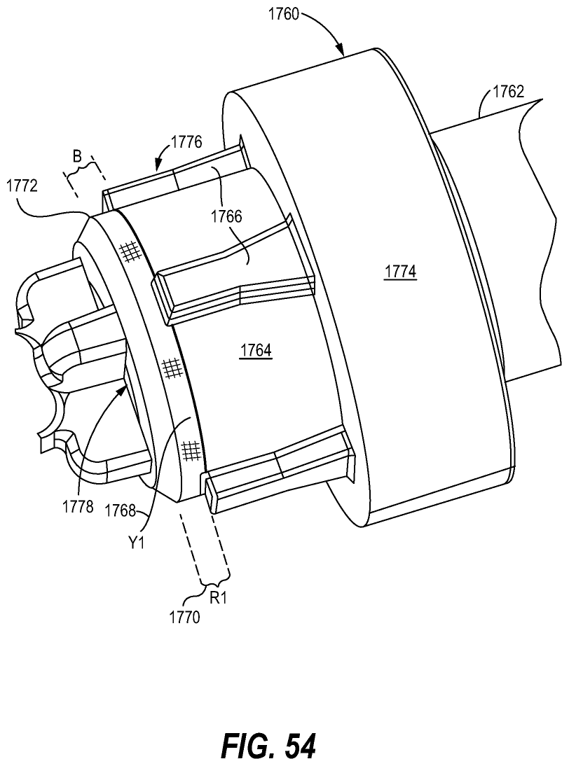

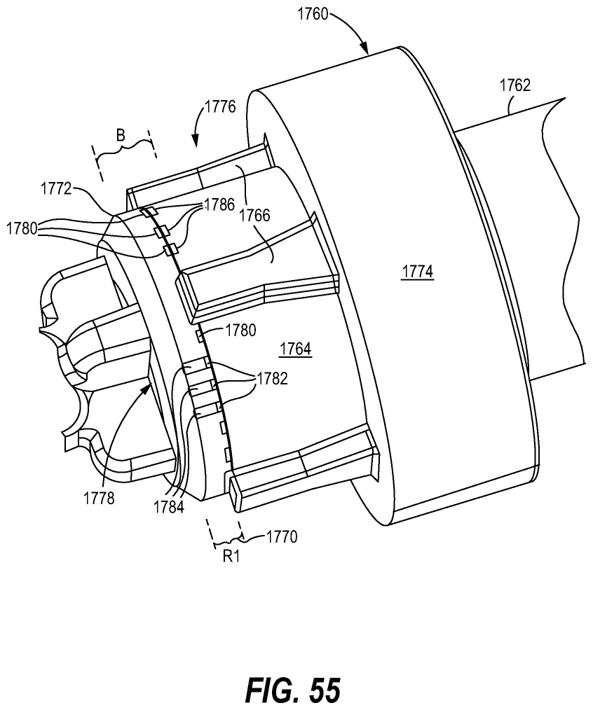

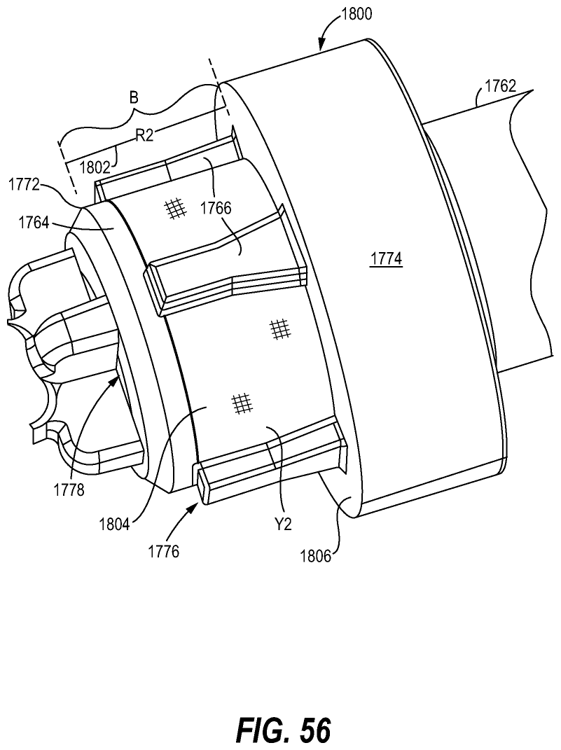

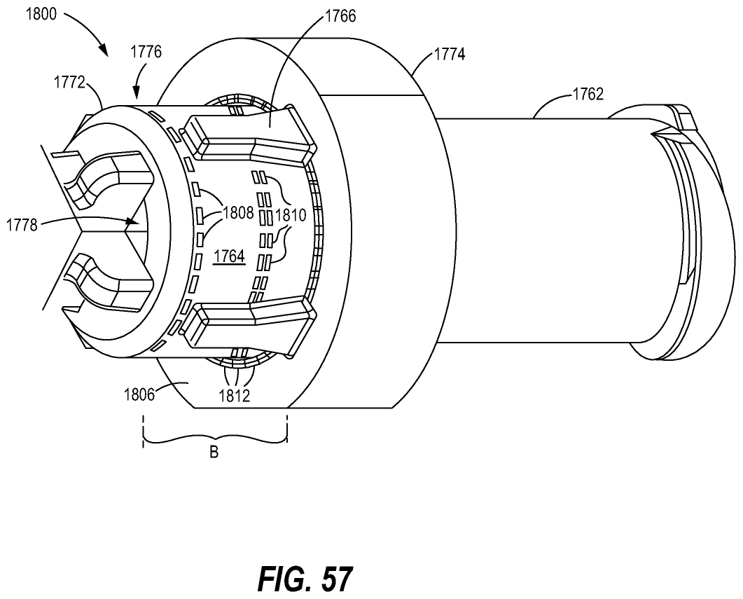

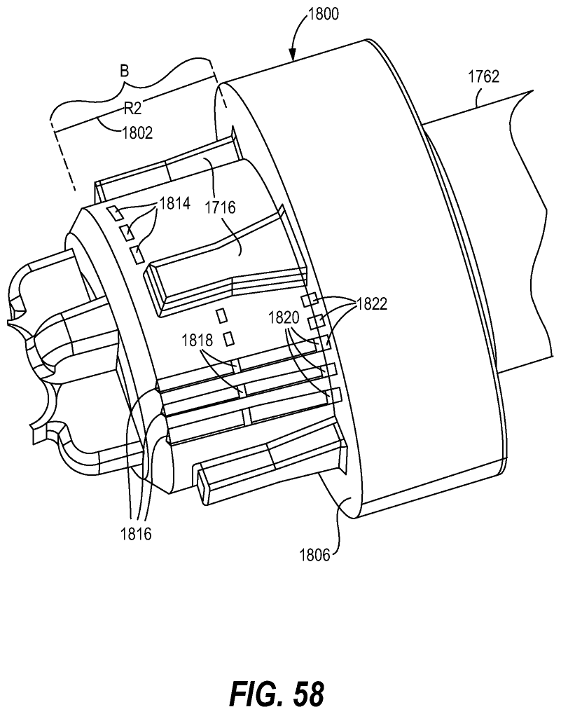

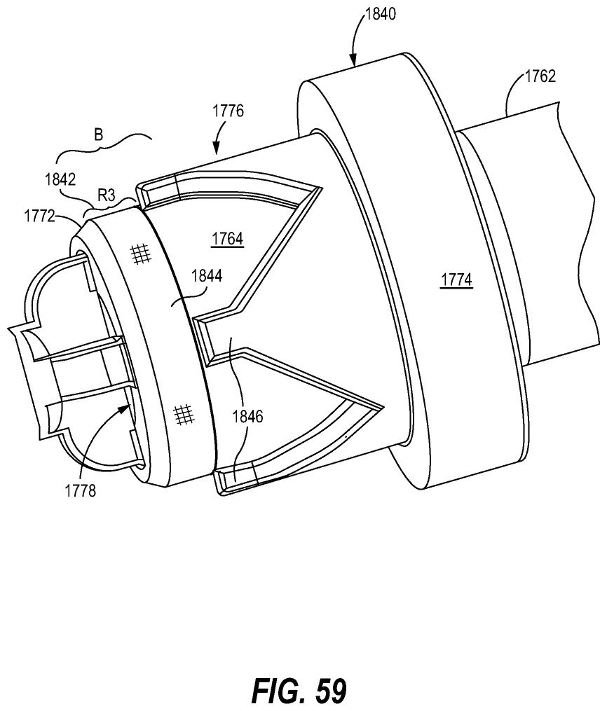

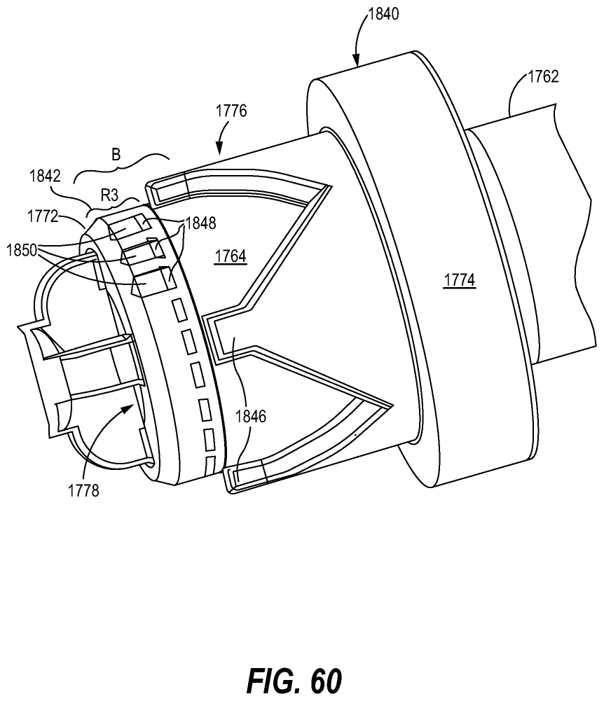

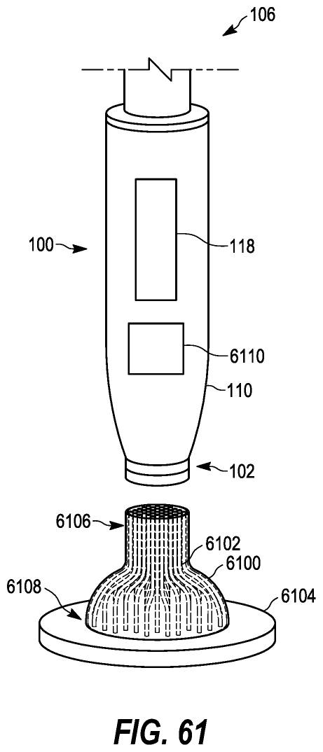

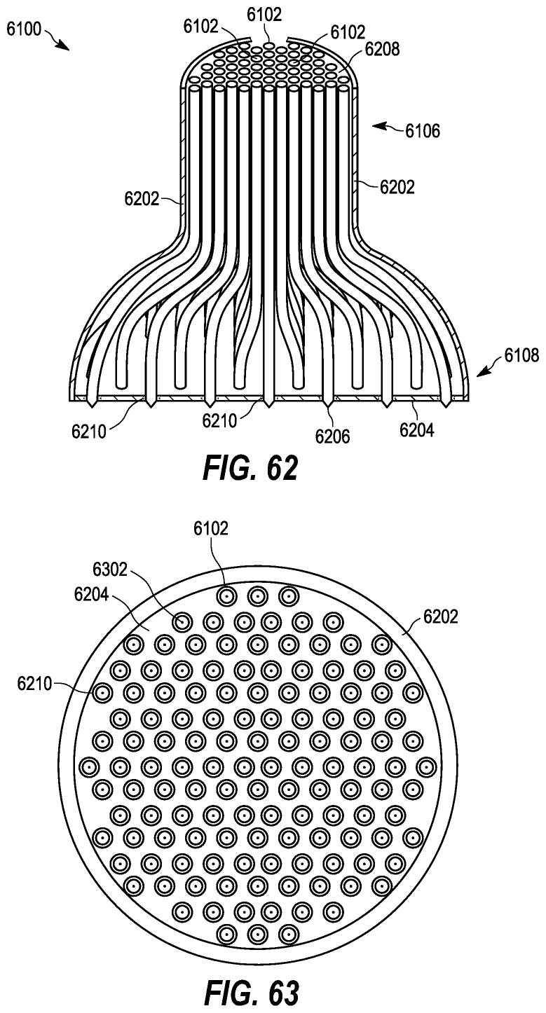

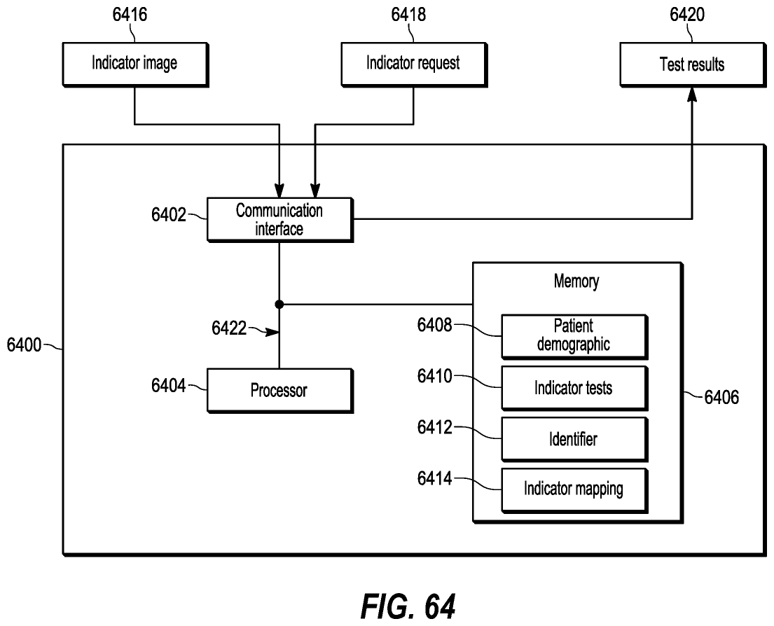

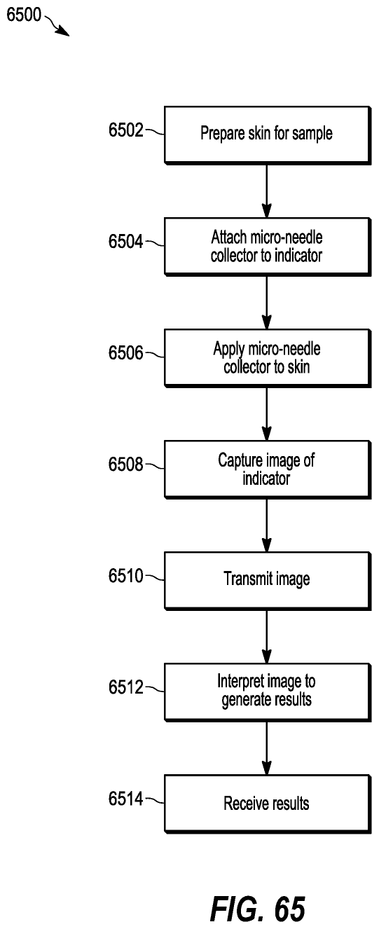

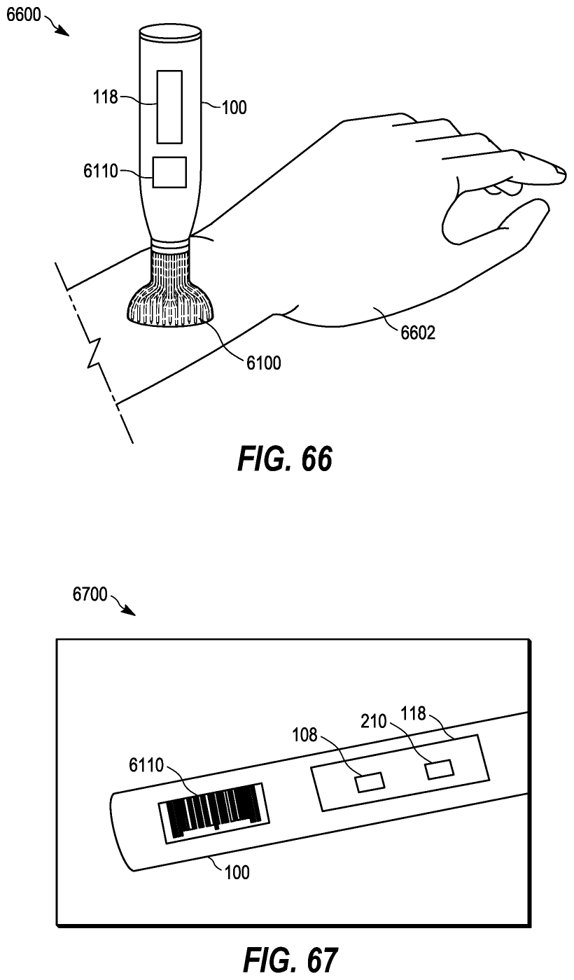

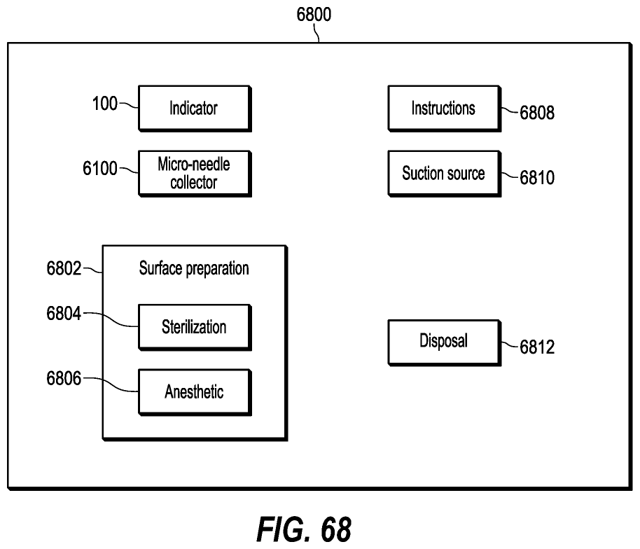

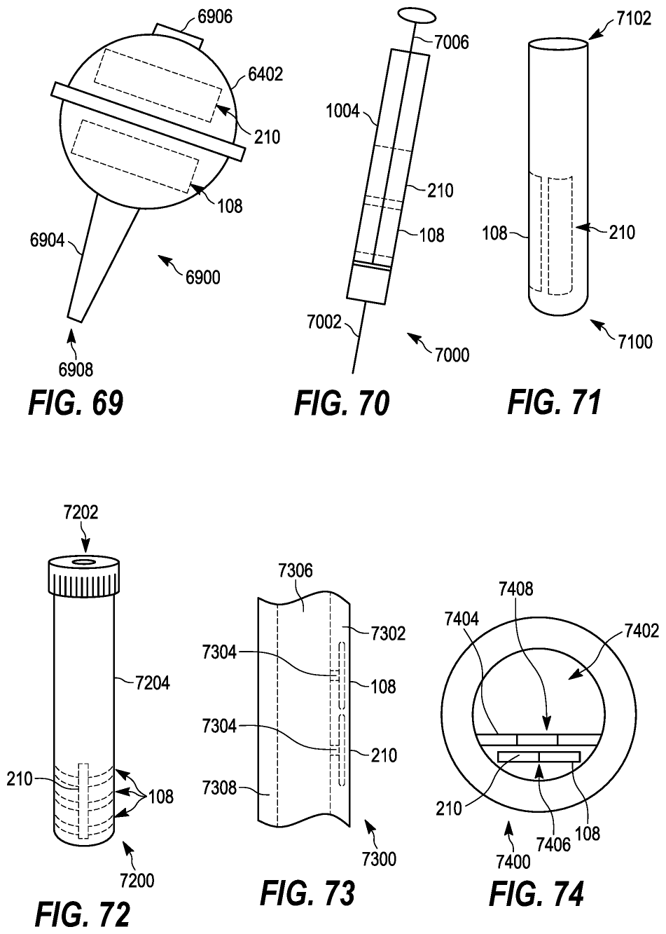

As best seen in FIGS. 50 and 51, there are shown schematically more general arrangements 5010, 5110 of first and second indicating elements or media, sources of fluid to be tested, and sources of suction, or the like, to cause the fluid to move toward and into contact with the indicating elements or media, which will be discussed before returning to discussion of the indicator 100 of FIGS. 1A-1D and to discussion of other embodiments.

As best seen in FIG. 50, a first example arrangement of elements 5010 comprises a source 5012 of fluid to be tested (e.g., as received from the distal end port 103), a first indicating element or medium 5016 (e.g., the first indicating element 108), a second indicating element or medium 5020 (e.g., the second indicating element 210), and a source of suction 5024 (e.g., as attached to the proximal end port 104) or the like to cause the fluid to move toward and into contact with the indicating elements or media. The source 5012 of fluid to be tested is coupled or in fluid communication with the first indicating element or medium 5016 (or a vessel containing it) via a fluid pathway, conduit, or channel 5014 (e.g., the passage 105). The first indicating element or medium 5016 (or the vessel containing it) is coupled or in fluid communication with the second indicating element or medium 5020 (or a vessel containing it) via a fluid pathway, conduit, or channel 5018. The second indicating element 5020 (or the vessel containing it) is coupled or in fluid communication with the source of suction 5024 via a fluid pathway, conduit, or channel 5022. As these components are arranged, operation of the source of suction 5024 may cause fluid from the fluid source 5012 to be drawn into contact with first indicating element or medium 5016 and then into contact with second indicating element or medium 5020.

As best seen in FIG. 51, a second example arrangement of elements 5110 comprises a source 5112 of fluid to be tested (e.g., as received from the distal end port 103), a first indicating element or medium 5116 (e.g., the first indicating element 108), a second indicating element or medium 5120 (e.g., the second indicating element 210) that may act as a usability/ready indicator, and a source of suction 5124 (e.g., as attached to the proximal end port 104) or the like to cause the fluid to move toward and into contact with the indicating elements or media. The source 5112 of fluid to be tested is coupled or in fluid communication with the first indicating element or medium 5116 (or a vessel containing it) via fluid pathway, conduit, or channel 5114, and with the second indicating element or medium 5120 (or a vessel containing it) via fluid pathway, conduit, or channel 5118. The source of suction 5124 is coupled or in fluid communication with the first indicating element or medium 5116 (or the vessel containing it) via a fluid pathway, conduit, or channel 5522, and with the second indicating element or medium 5120 (or the vessel containing it) via fluid pathway, conduit, or channel 5122. As these components are arranged, operation of the source of suction 5124 may cause fluid from the fluid source 5112 to be drawn into contact with first indicating element or medium 5116 and with second indicating element or medium 5120 either simultaneously or with no expectation as to the order in which contact occurs.

As best seen in FIGS. 50 and 51, the sources 5012 and 5112 of fluid to be tested may be represented generally using the reference symbol “F”. The fluid sources may be, as non-limiting examples, a medical or veterinary patient or a fluid-containing organ thereof, or may be a sample container, or another other container or vessel.

The sources of suction 5024 and 5124 may be represented generally using the reference symbol “S”. The suction sources may be, as non-limiting example, a domestic suction or vacuum system that might be found in a medical office or hospital, a vacuum pump, a suction bulb, a syringe, an evacuated test tube, or the like. Alternatively or additionally, other means to cause fluid to move from the fluid sources toward and into contact with the indicating elements or media may be used. For example, the pressure may be applied at the fluid source via a source of air or other compressed gas, a syringe or bulb, a pump, or similar apparatus.

Responsive to the contact with or the presence of the fluid, the first indicating elements or media 5016 and 5116, may exhibit an externally observable indication, or change therein, relating to respective fluid characteristics or properties to be sensed or measured. For example but without limitation, the indicating elements 5016 and 5116 may be constructed of a pH-indicating paper which presents a color or shade or other visible indicia corresponding to the pH of the fluid, or a range thereof, or the value of the characteristic on one side or the other of a defined threshold. Indicating elements or media of other types, or sensitive to other fluid characteristics or properties, could also be used, as discussed further.

Responsive to contact with or the presence of the fluid, the second indicating elements or media 5020 and 5120, may exhibit an externally observable indication, or change therein, relating to respective fluid characteristics or properties that are sensed or measured which provide information to an observer regarding whether one or more indications provided by the first indicating elements or media 5016 and 5116 are suitable or ready for use. The first and second indicating elements or media 5016 and 5020 or 5116 and 5120 may be integrated in a single indicator package or housing as shown by brackets 5030 and 5130, but could also occupy separate indicator housings, containers, or the like. Where the first and second indicating elements or media 5016 and 5020 or 5116 and 5120 are integrated in a single indicator package or housing 5030 or 5130, fluid pathways, conduits, or channels between and around them, such as 5014, 5018, 5022, 5114, 5118, 5122, and 5126 may be, at least in part, contained within or formed by such package or housing.

Thus, the first indicating element 5016, 5116 may be considered to be a detection indicator, and the second indicating element 5020, 5120 may be considered to be a usability or ready indicator, with respect to an indicator device of which they may be a part or otherwise associated.

Suitability or readiness for use depends on several factors, including without limitation, the application environment, the type of fluid being tested, the characteristic or measurement intended to be made by the first indicating elements or media, the particular types of first indicating elements or media being used, environmental or other exposures to which the first indicating elements or media might be subject during storage or preparation for use, and the like. These factors may also determine whether the first schematic arrangement 5010 of elements of FIG. 50, the second schematic arrangement 5110 of FIG. 51, some modification of these, or another arrangement, are suitable for use.

For example, if the first indicating element 5016, 5116 may be damaged by prior exposure to water, such that a subsequent measurement or indication would be unreliable, the second indicating element 5020, 5120 may be a water detecting material that changes color permanently upon exposure to water. In that case, the effective operation of the second indicating element 5020, 5120, at the time an indicator device is used, may not depend on the order in which the fluid under test exposes the first and second indicating elements, and therefore either of arrangements 5010 or 5510 could be used to enable the indication of the second indicating element to warn an observer that the indication from the first indicating element is unreliable. Example commercial products suitable for implementing the second indicating element in applications where the second indicating element is intended to signal prior contact with water include water contact indicator tapes offered under the product numbers 5557, 5557NP, 5558, 5559, and 5559i by the 3M Company of St. Paul, Minn.

For another example, if it were necessary that the first indicating element 5016, 5116 be adequately exposed to or saturated by the fluid to be tested before its indication may be relied upon by a user, the second indicating element 5020, 5120 may be sensitive to the same fluid as the first indicating element. In that case, the order of fluid exposure may be important, and arrangement 5010 could be used, whereby the arrangement ensures that the second indicating element 5020 is not exposed to fluid until the first indicating element 5016 has been adequately exposed, and the indication of the second indicating element 50020 would inform an observer that the indication from the first indicating element 5016 may reliably be used.

Thus, the structure, composition, and materials of the first and second indicating elements could be identical or different. The first and second indicating elements may be differentiated, for non-limiting examples, by their locations or placement in the fluid path or in an indicating device, or by color, shape, and sensitivity to different fluid characteristics or properties.

Any appropriate chemical-property indicating devices, media, measuring components, or substances including but not limited to litmus, pH indicating strips, paper, cloth, or any other substrate impregnated with or bearing a pH indicator, or the like, may be used to implement the indicating elements 5016, 5020, 5116, and 5120.

The indicators disclosed herein, including without limitation indicator 100, may generally be used to obtain a measurement of the gastric pH. This measurement may be employed for purposes in addition to establishing correct insertion of a nasogastric tube, including, without limitation, determination that the stomach is prepared to receive a therapeutic agent, or that an appropriate quantity of a therapeutic agent affecting pH, has been introduced. As an alternative to a pH-sensitive medium, the first and second indicator elements may be implemented using media indicating chemical properties other than pH, which may verify correct insertion of a nasogastric tube, signal incorrect insertion of a nasogastric tube, or verify correct or sufficient introduction of a therapeutic, buffering, or irrigation agent. The indicators and the first and second indicating elements may be used with fluids other than gastric aspirates, including without limitation blood, plasma, and gases, and may be used with such fluids including particulates, granules, powders and other solids that may be entrained therein.

Any of the indicating elements, and the measuring components, substances, or media thereof, described for use with the indicators herein, may be realized using any suitable paper, material or device which can measure, sense, or detect a characteristic or property of a fluid and provide a visual or other externally discernible indication of the measurement. In this description of the properties of the indicating elements, it is intended that references to the indicating elements generally also include the measuring components, substance, or media thereof. Moreover, the indicating elements may include without limitation various configurations wherein the indicating element is: (a) formed as a substrate or matrix, which may or may not be permeable, with the measuring components, substance, or media impregnated, entrained, enmeshed, combined, or otherwise disposed therein; (b) formed as a substrate, with the measuring components, substance, ink, matrix, or media applied to, attached to, adhered to, printed on, or otherwise disposed on a surface or embedded in a matrix thereof; or (c) formed as a structure substantially composed of the measuring components, substance, or media, e.g., as a solid, matrix, or gel. The measuring components, substances, or media may be solid or flexible and may be dry or remain wet or tacky during the shelf life of commercial embodiments. Wet or tacky measuring components may be needed or desirable for use in measuring characteristics of particulates, granules, and powders. The indicating element preferably furnishes a visual indication of a chemical property, such as pH, which may, for example, be manifested as a change in color, reflectivity, or the like. The indication may, for example, be represented as a distinguishable one of two well-defined states (for example, the indicator might be red in one state blue in another), or a distinguishable one of a larger plurality of recognizable discrete states, or as an observable point, value, or range along a continuum (or example, the indicator might display a shade of color, within a range of colors).

The first and second indicating elements or media may also be sensitive to other properties, such as the presence or activity of certain proteins, such as a paper with antibodies that react or provide an indication responsive to the presence or absence of the proteins. Any protein for which an indicative antibody or other sensing substance is available could be the target of the indicating element. For one non-limiting example, the indicator may sense the presence of Troponin in concentrations above a threshold. Troponin is a protein found in higher concentrations during a myocardial infarction, and thus, an indicator equipped with an indicating element sensitive to Troponin could be useful clinically to provide a rapid determination of that condition, allowing for more immediate treatment. Moreover, the first and second indicating elements or media may be sensitive to other substances or properties, including without limitation, genetic markers, toxins, drugs (e.g., acetaminophen), albumin, Human Chorionic Gonadotropin (HCG), any substance for which an immunoassay is available, or any other substance or property.

The first and second indicating elements or media may be adapted to be sensitive to selected characteristics or properties of the fluid, regardless of the presence or absence of other materials, chemicals, or agents in the fluid. Some commercially-available indicating media, including some commercial test strips, are sensitive to or present reliable indications as to a sensed or measured characteristic of a fluid only when certain other accompanying chemicals or agents are present. For example, some test strips operate correctly only when the fluid under test is buffered or is accompanied by certain classes of organic molecules. In applications where the indicator is expected to operate reliably even if these conditions may not be satisfied, the indicating element should be selected to avoid those whose correct operation relies on these conditions. In some cases, an ordinary indicating element or medium may be used as the first indicating element or medium, and an indicating element or medium selected to avoid the buffer or accompanying organic molecule requirements may be used as the second indicating element or medium, or vice versa.

The first and second indicating elements or media may furnish a simple indication, such as a color, throughout such indicating elements or an exposed or saturated part thereof. The indicating elements may also display shapes, letters, icons, symbols, or other indicia in addition to or instead of colors, and the measurement or indication may by represented or distinguished by position, orientation, or quantity of indicia, or some visible change in appearance (or other detectable change) of the indicating elements. For non-limiting examples, the indication or measurement may be provided as the appearance (or absence) of a word or phrase (e.g., “OK”, “STOP”, etc.), the quantity of visible symbols, the quantity of symbols which are a particular color or have undergone a change in color, the highest or lowest of several displayed numbers, the highest or lowest of several numbers which have undergone a change in color, a recognizable pattern of indicia, or the like.

Either or both of the first and second indicating elements may be provided in plurality. The indicating elements may also bear a plurality of measuring components, substances, or media. The plurality of measuring components, substances, or media may be sensitive to different respective properties or substances. It may be desirable to have plural first indicating elements, e.g., of the same type, at diverse locations to provide additional opportunities for exposure and observation, in a device. It may also be desirable to have plural first indicating elements of differing types or sensitivities to different fluid properties or characteristics. These may be juxtaposed or organized in groups or patterns so as to present measurements by way of combinations or cooperation of several indicia or components. Thus, different measuring components, substances, or media may overlap or be adjacently arranged, so that the measurement indication is presented as a composite or blended color, or distinguishable patterns or symbols, or the like. Other arrangements could also be used.

It may likewise be desirable to have plural second indicating elements, as needed, to provide information as to whether any one or more of the indications from one or more first indicating elements are suitable for use. For example, if multiple first indicating elements are provided, having sensitivities to different fluid characteristics, multiple second indicating elements may also be needed to inform a user or observer that the indications from the first indicating element are (or are not) suitable for use. However, even if plural first indicating elements are used, a single instance of the second indicating element may suffice to inform the user or observer whether the indications from the first indicating elements are suitable for use. Accordingly, although each of the first indicating element and the second indicating element is generally hereinafter referred to in the singular, it will be appreciated that such references may include a group of such elements.

The first and second indicating elements may be different segments or regions of a single object, such as a strip of indicating paper.

Although the indicating elements are generally described herein as presenting a visible indication, they (or any of them) may also or instead be adapted to produce an indication suitable for sensing, measurement or detection by a machine or other apparatus, and such indications might not be visible or usefully discernible by a human observer. For example, either of the first or second indicating elements, or a combination of the first and second indicating elements, may be able to be read by a bar code machine. The bar code machine could scan the indicating elements and provide the results to the medical records of the patient. For example, it may be an electronic medical record that is updated with the results of the bar code scan, or it may cause a print-out of the results to be inserted into a paper copy of the medical records of the patients. Further, a colorimeter or a colorimetric machine may be able to discern the color of the first and/or second indicating elements and similarly update a patient's medical records. Additionally, responsive to determining that the second indicating element indicates a ‘do-not-use’ reading, the bar code scanner may be configured to alert the user via a visual or audible indication.

The reference symbol “A” is used in the Figures to denote instances of the first indicating element or medium (or segment or group thereof), such as 5016 or 5116, or a zone, section, or region in which such instance resides. The reference symbol “B” is used in the Figures to denote instances of the second indicating element or medium (or segment or group thereof), such as 5020 or 5120, or a zone, section, or region in which such instance resides.

Several configurations of the first and second indicating elements or media will be described herein and shown in the Figures. The reference symbol “X” is used in the Figures generally to denote one or more instances of the second indicating element where the second indicating element takes a form similar to that of the first indicating element, and is located further from the source of fluid being tested than the first indicating element. (The term “further from the source of fluid being tested” refers to a relative location along the path of fluid flow.) The second indicating element 210 of FIGS. 1A-1E is an example of such an instance, where each of the first and second indicating elements 108 and 210 are formed as a tube. First and second indicating elements 108 and 210 could, for example, be tubes constructed of a paper indicating medium, longitudinally end-wise arranged. First and second indicating elements 108 and 210 could also be longitudinally-adjacent portions of a single tube constructed of a paper indicating medium. First and second indicating elements 108 and 210 may be sensitive to the same properties or different properties of a fluid being tested.

As best seen in FIGS. 1A-1D, indicator 100 preferably comprises a housing 110 and a diffuser element 120, a portion of which is telescoped into the housing 110. FIG. 1E is a partial exploded view depicting the housing 110 and diffuser 120 components of indicator 100 separately, for example, as they might appear prior to assembly as a unit, by telescoping a distal end 148 of diffuser 120 into a housing proximal-end opening 152 at the proximal end 150 of the housing as shown by broken line 101. Although an embodiment of indicator 100 may be constructed by so assembling the housing 110 and diffuser 120, indicator 100 could also be constructed in other ways that may not require the housing 110 and diffuser 120 to exist as separate elements which are later joined. The subsequent description of the indicator 100 may best be understood by considering, in conjunction, FIGS. 1A-1E.

Housing 110 is constructed as a generally tubular or barrel shaped body, which has a distal end 112, and a proximal end 150. The housing 110 has, adjoined in series from proximal end 150 to distal end 112, a first section 156, a second section 158 including the indicator window 118, a third or central section 122, and a fourth section 160. These sections are not necessarily separate parts or components, although they could be, and are identified for ease of reference to structure or function. The housing has a distal opening or intake port 113 at the distal end 112, and a housing proximal-end opening 152 at the proximal end 150, which are joined by an interior central fluid communication chamber, lumen, or housing fluid passage 138 extending generally longitudinally between the ports 152 and 113. In the assembled form of indicator 100, housing fluid passage 138 serves as the distal portion the fluid passage 105, and housing distal port 113 serves as the intake distal port 103. The housing fluid passage 138 may include various volumes, spaces, conduits, channels, chambers, and the like in fluid communication within the housing 110, and may include all or less than all of the open interior space in the housing not occupied by solid materials.

Housing 110 preferably has a first diameter at first section 156 and second section 158 to receive a portion of diffuser 120. Housing 110 may have at fourth section 160 and distal end 112 a second diameter, relatively smaller than the first diameter. The diameter of fourth section 160 and distal end 112, and the shapes thereof, preferably allow fourth section 160 to mate, couple, or connect with a fluid transport source (not shown), which may take the form of tubing, conduits, adapters, appliances, containers, reservoirs, catheters, feeding tubes, suction tubes, or the like, which may be used in a clinical environment to furnish the fluid with which indicator 100 is to be used to perform a test, or obtain a measurement, or indication of one or more characteristic or property of the fluid. For example, the fourth section 160 and distal end 112 may have a diameter and be formed with a “catheter tip” shape suitable for mating, coupling, or connection with a catheter, such as a nasogastric tube or feeding tube which is inserted into a patient's stomach and which serves as the fluid transport source. The catheter tip can also be mated with other types of receiving connector, plugs, hubs, and the like.

Because vacuum or suction will typically be used to draw or aspirate fluid from the fluid transport source into the indicator 100, the mating, coupling, or connection between housing 110 and the fluid transport source preferably forms a substantially vacuum-tight seal at the vacuum or suction pressures used in a clinical environment. The indicator 100 could also be used in a mode where fluid under test is furnished from a source under some pressure, in which case the seal is preferably sufficient to avoid leakage of the fluid. The housing 110 or portions thereof, such as fourth section 160, which will mate or couple with a fluid transport source or adapter, may have a surface texture or surface treatment 142 that forms or enhances the seal. For example, the surface treatment 142 may be a flexible, resilient, or tacky coating that grips or adheres to a mating surface, such as the inside diameter, of the fluid transport source. Surface treatment 142 may also be implemented as a pattern or texture on or in the surface of housing 110 that increases adhesion or local contact pressure between housing 110 and the fluid transport source, or otherwise enhances the seal. For example, the surface treatment 142 may be constructed as one or more circumferential ridges disposed near distal end 112, but could also be any other suitable pattern or texture, including hatching, a random or pseudo-random texture, geometrical shapes, or the like.

Although housing 110 is depicted in FIGS. 1A-1E, with a “catheter tip” distal end 112 configuration that accommodates frictional, sealing engagement with the fluid transport source, housing 110 could also have another structure for coupling to the fluid transport source. FIG. 2A is a side view of an embodiment 170 of a housing constructed in accordance with a further aspect of the present invention. Housing 170 is generally similar to the housing 110 of FIGS. 1A-1E, but has a male lock fitting 174 (e.g., a Luer lock type fitting) at the distal end 172, instead of the catheter tip. Housing 170 is generally similar to housing 110 of FIGS. 1A-1E as heretofore described, and except for the differences explained below, the foregoing description of housing 110 housing 170, mutatis mutandis, and is hereby incorporated by reference. The lock fitting 174 may mate with a complementary fitting on the fluid transport source. The positive mating avoids the need to rely on friction alone for a seal and may avoid malfunction or spills when tension applied between the indicator 100 and the fluid transport source may tend to separate these components. Any other appropriate coupling or fitting could also be used. Thus, in constructing an embodiment of indicator 100, housing 170 of FIG. 2A may be substituted for housing 110.

Alternatively, an adapter, such as the adapter 700 of FIGS. 7A-7G, discussed further below, could be used to make the coupling between distal end 112 and a fluid transport source having a Luer lock or similar fitting.

As a further alternative, any of the housings described herein could have a port with an appropriate puncturable seal for use with a sample collection appliance of the type typically used in clinical settings for collection of blood samples. Such appliances typically have a cup-shaped receptacle with a two-ended hollow needle extending in both directions from the closed end of the cup. In typical use in collecting a blood sample from a patient, the outward-extending end of the needle is inserted into the patient's vein. An evacuated test tube with a puncturable seal is then inserted, seal-end first, into the cup, so that the inward-extending end of the needle punctures the seal. Because the test tube is evacuated, blood is drawn through the needle into the test tube. When an adequate sample of blood has been acquired, the test tube is removed from the cup, and the seal recloses around the puncture. The appliance may then be removed from the patient. A similar seal could be provided on the distal end of any of the housings described herein, so that the associated indicator may be used with the aforementioned sample collection appliance in the same manner as a sample test tube. Moreover, the proximal end port may be sealed or omitted, and the indicator could be evacuated, so that when the indicator is inserted into the cup of the sample collection accessory, puncturing the seal, the partial vacuum draws blood through the needle into the indicator, without requiring an external source of vacuum or suction.

As best seen in FIGS. 1A-1E, third or central section 122 may provide a tapering transition from the smaller diameter of the fourth section 160 to the larger diameter of the second section 158 and the first section 156. The section of the housing fluid passage 138 that extends through third or central section 122 may also have an interior diameter that is similarly tapered, accommodating the tapered shape of the tip section 162 of diffuser 120 which extends through the housing fluid passage 138 in the area of the central section 122. As will be described further in greater detail, among the functions of the diffuser 120 are to control the distribution of fluid that enters the indicator 100 at the distal end port 103, 113 to the first and second indicating elements 108 and 210. In some cases, the diffuser 120 may have one or more of these functions: to divide, render more uniform or otherwise optimize or improve the fluid distribution to one or more of the indicating elements 108 and 210; to increase contact of the fluid with one or more of the indicating elements 108 and 210; to increase exposure or saturation of one or more of the indicating elements 108 and 210 with the fluid; to cause exposure or saturation to the entirety of the first indicating element 108 to occur at approximately the same time or nearly simultaneously; to cause exposure or saturation of the entirety of the second indicating element 210 to occur simultaneously; to cause exposure or saturation of both of the first and second indicating elements 108 and 210 to occur simultaneously, or in a controlled progression from distal to proximal ends, to the greatest extent possible; and/or to filter the fluid so as to exclude or preferentially route or direct components thereof toward the indicating element. The diffuser 120 may have additional functions. In an example embodiment of the housing 110 constructed according to an aspect of the invention, the portion of the housing fluid passage 138 in the area of central section 122 is a generally coaxial channel formed as the space between the tapered diffuser tip section 162 and the interior surface of central section 122. In some embodiments the diffuser 120 may act as a filter or may hold the indicator in place. It may be connected or in two separate parts. In some embodiments, the filter may be the filter paper that also houses the secondary indicator.

The second section 158 of housing 110 preferably contains an indicator window 118 which is substantially transparent to allow observation of the first and second indicating elements 108 and 210 disposed in the interior of housing 110. As described below in greater detail, in some embodiments, the first and second indicating elements 108 and 210 may be tubular or cylindrical and may be generally coaxial with the body of housing 110 and the diffuser 120, in which case it may be preferable that the indicator window 118 allow observation at any angle about the general longitudinal axis of the indicator 100. The indicator window 118 may, for non-limiting example, be realized by constructing section 158, or some longitudinal extent thereof, from a transparent material, which would afford observation from any angle about the longitudinal axis. However, in some embodiments, the indicating elements 108 and 210, or the measuring, sensing, or indicating part thereof, may be occupy a full circumference around the general longitudinal axis. For example, one of more of the indicating elements 108 and 210 could be constructed as a strip or dot of indicating medium (or plurality of them) that appears only on one side (or some other limited angular extent) of diffuser 120. In that case, section 158 may have a smaller indicator window 118 configured to ensure that the location of the indicating elements 108 and 210 are conspicuously visible through the indicator window 118, to avoid erroneous readings that could occur if the user mistakes some other internal component as the indicating elements 108 and 210. In some cases, fluid or air flow through the device, or handling of the device, may tend to dislodge any of the indicating elements from a preferred location with respect to the indicator window or other elements. It is generally desirable that the indicating element not move when the indicator is in use. A retaining shoulder 184 of reduced diameter (i.e., extending inward) may be provided on the interior surface of the housing 110 near the distal end of the first section 156 to prevent undesired migration of the indicating element 210. If the indicating elements 108 and 210 are implemented using a single physical structure, such as a paper tube impregnated with indicating chemicals or the like, a single retaining shoulder 184 will have the effect of retaining that structure and will retain both indicating elements. If the indicating elements 108 and 210 are implemented using separate physical structures, additional retaining shoulders (not shown) may be required to keep each indicating element in a preferred location. The retaining shoulder 184 may, for example, have a radial thickness of approximately 0.010 in. The indicating element could also be secured or fixed in location with respect to the housing using other structures such as tabs, clips or burrs, adhesive attachment, other chemical attachment means, thermal, chemical, or ultrasonic welding, or any other appropriate securement means. The retaining shoulder 184 or other indicating element retaining means may be used on any of the housings described herein.

A port 136 may be provided on a portion of the housing 110 to allow fluid to be withdrawn, e.g., for other testing, examination, treatment, diagnosis, or procedures. The port 136 also allows reagents, dies, other chemicals, or other material to be added. This could be used, for example, where the indicator may furnish a measurement only with the added material, or where the indicator may furnish an additional useful measurement, or additional precision in measurement, after the added material. The port 136 may be a puncturable seal, so that material may be added or removed via a needle and syringe or the like, and the seal may be a self-reclosing seal to avoid leakage in or out through the port after use.

As best seen in FIG. 1A2, there is shown an example indicator 212 which employs a further example configuration of first and second indicating elements. Indicator 212 may be constructed in a manner similar to indicator 100 of FIGS. 1A-1F, unless otherwise described, and like reference numbers in FIG. 1A2 refer to equivalent items in FIGS. 1A-1F. A first indicating element 214 of generally tubular form, similar to the first indicating element 108 of FIGS. 1A-1F is provided. A second indicating element 216 is formed as a strip or section extending longitudinally along the length of the first indicating element 214. The second indicating element 216 may, for example, be a defined portion of the tubular first indicating element 214. Alternatively, a portion of the first indicating element 214, corresponding to the location of the second indicating element 216, may be omitted, and the second indicating element 216 may be provided as a separate strip of indicating media. Alternatively, the first indicating element 214 may be provided as a complete tube, and the second indicating element 216 may be provided as a longitudinal strip on or near the outside of the first indicating element 214 so as to visually overlap the first indicating element when viewed from the exterior of the indicator. The reference symbol “W” is used in the Figures generally to denote one or more instances of the second indicating element where the second indicating element extends longitudinally along most or all of first indicating element. Due to substantial symmetry about the longitudinal axis of the indicator 212, fluid is likely to arrive at and expose the first and second indicating elements 214 and 216 at about the same time. This configuration of first and second indicating elements corresponds to the second schematic arrangement 5110 of FIG. 51.

As best seen in FIG. 1A3, there is shown another example indicator 220 which employs a further example configuration of first and second indicating elements. Indicator 220 may be constructed in a manner similar to indicator 100 of FIGS. 1A-1F, unless otherwise described, and like reference numbers in FIG. 1A3 refer to equivalent items in FIGS. 1A-1F. A first indicating element 224 of generally tubular form, similar to the first indicating element 108 of FIGS. 1A-1F is provided. A second indicating element 226 is formed as a small indicating segment of any suitable shape near the downstream end of the first indicating element 224. The second indicating element 226 may, for example, be a formed as a small dot. The second indicating element 226 may be a defined portion of the tubular first indicating element 224. Alternatively, a portion of the first indicating element 224, corresponding to the location of the second indicating element 226, may be omitted, and the second indicating element 226 may be provided as a separate piece or strip of indicating media. Alternatively, the first indicating element 224 may be provided as a complete tube, and the second indicating element 226 may be provided as a piece or strip of indicating medium on or near the outside of the first indicating element 224 so as to visually overlap the first indicating element when viewed from the exterior of the indicator. The reference symbol “L” is used in the Figures generally to denote one or more instances of the second indicating element where the second indicating element is a small element or segment near the downstream end of the first indicating element. Because the second indicating element 226 is near the downstream end of the first indicating element 224, fluid is likely to arrive at and expose at least a portion of the first indicating element 224 before the fluid arrives at the second indicating element 226. This configuration of first and second indicating elements corresponds to the first schematic arrangement 5010 of FIG. 50.

As best seen in FIG. 1A4, there is shown another example indicator 230 which employs a further example configuration of first and second indicating elements. Indicator 230 may be constructed in a manner similar to indicator 100 of FIGS. 1A-1F, unless otherwise described, and like reference numbers in FIG. 1A4 refer to equivalent items in FIGS. 1A-1F. A first indicating element 234 of generally tubular form, similar to the first indicating element 108 of FIGS. 1A-1F is provided. A second indicating element 236 is formed as a cylindrical or tubular band near the downstream end of the first indicating element 224 and radially near or adjacent an inner surface 238 of housing 230. Second indicating element 236 may, for example, be a discrete piece of indicating medium disposed within housing fluid passage 138. Alternatively, second indicating element 236 may be a discrete piece of indicating medium applied or affixed to the inner surface 238 of housing 230, using an adhesive. Alternatively, second indicating element 236 may be an indicating-medium coating applied to the inner surface 238 of housing 230. The radial position of second indicating element 236 outward of the first indicating element 234 causes the second indicating element 236 to overlap the first indicating element when viewed from the exterior of the indicator. The reference symbol “M” is used in the Figures generally to denote one or more instances of the second indicating element where the second indicating element radially disposed outward of the first indicating element and located downstream with respect to the first indicating element. Because the second indicating element is near the downstream end of the first indicating element, fluid is likely to arrive at and expose at least a portion of the first indicating element 234 before the fluid arrives at the second indicating element 236. This configuration of first and second indicating elements corresponds to the first schematic arrangement 5010 of FIG. 50.

According to a further aspect of the invention, the housing central fluid communication chamber or passage may have a modified cross section to cooperate with the diffuser 120 to distribute fluid to the indicating elements 108 and 210.

FIG. 2B is an isometric view, and FIG. 2C is a side view, of a further embodiment 190 of a housing which is similar to the housing 110 of FIGS. 1A-1E but which provides a central fluid passage 192 of modified cross section. Housing 190 is generally similar to housing 110 of FIGS. 1A-1E as heretofore described, and except for the differences explained below, the foregoing description of housing 110 is applicable to housing 190, mutatis mutandis, and is hereby incorporated by reference. A fluid passage 192, which functions in a manner similar to the housing fluid passage 138 of the housing 110 of FIGS. 1A-1E, extends between a distal-end or intake port 198 and a proximal end opening 188. FIG. 2D is a view of housing 190 looking into the proximal end opening 188 as shown by view lines 2D-2D of FIG. 2C. FIG. 2E is a view of housing 190 looking into the distal end opening 198 as shown by view lines 2E-2E of FIG. 2C. FIG. 2F is a cutaway view of the housing 190 of FIGS. 2B and 2C, taken along the view lines 2F-2F of FIG. 2C.

As best seen in FIGS. 2B, 2D, and 2F, housing 190 has a plurality of inward-facing ribs 196 extending radially inward into fluid passage section 192, and extending longitudinally along at least a portion of the housing 190. The longitudinal ribs 196 form a plurality of complementary longitudinally-extending fluid channels 194. The diffuser tip section 162 and the longitudinal ribs 196 may be sized to substantially fill the fluid passage 192 except for the fluid channels 194, to force all the fluid to pass through the fluid channels 194. Alternatively, the diffuser tip section 162 and longitudinal ribs 196 may be sized to allow space therebetween, providing as part of fluid passage 192 an additional coaxial channel for fluid flow that extends longitudinally along housing 190. These geometries and spacing may be adapted to accommodate expected characteristics of the fluid, such as viscosity, the presence of solids, or the like. In constructing an embodiment of indicator 100, housing 190 may be substituted for housing 110. The configuration of the fluid channels 194 tends to promote laminar flow of the fluid as it approaches the first and second indicating elements 108 and 210. Laminar flow may help regulate suction, and may help control the consistency or filtration of the fluid or its components. In addition, laminar flow can reduce the risk of washing off or otherwise removing components of the indicator element, such as dies, and can reduce the risk of damaging the indicator element, which may be fragile. All of these risks may be higher when turbulent fluid flow, as opposed to the laminar flow, is present.

As best seen in FIGS. 1A-1E, diffuser 120 has a proximal end 146 and a distal end 148. In some embodiments according to an aspect of the invention, indicator 100 is assembled by telescoping the distal end 148 of the diffuser 120 into the proximal-end opening 152 of housing 110. In those embodiments, the diffuser 120, or one or more portions thereof, functions as a closure or end cap for the housing 110, and further provides a proximal-end opening or outflow port 114 into a fluid communication chamber, lumen, or fluid passage 115. In the assembled configuration of indicator 100, diffuser fluid passage 115 serves as the proximal portion of the fluid passage 105, and the housing proximal-end port 114 serves as the outflow port 104 of the indicator 100. The diffuser fluid passage 115 may include various volumes, spaces, conduits, channels, chambers, and the like in fluid communication within or adjacent to the diffuser 120. In the assembled configuration of indicator 100, the housing proximal-end port 114 is in fluid communication via the diffuser fluid passage 115 with the interior volume and housing fluid passage 138 of housing 110. In typical operation, the housing proximal-end port 104, 114 is connected to a source of suction or vacuum so as to draw fluid under test through distal end port 103, 113, housing fluid passage 138, and into the region of the indicating elements 108 and 210. Port 104, 114 preferably has an appropriate fitting 134 for secure connection to a tube or conduit coupled to a source of suction or vacuum. For example, fitting 134 may be male Luer lock fitting. Other fittings could also be used.

In some cases, the diffuser 120 may have one or more of these functions: to divide, render uniform or otherwise improve the fluid distribution to one or more of the indicating elements 108 and 210; to increase contact of the fluid with one or more of the indicating elements 108 and 210; to increase exposure or saturation of as much of one or more of the indicating elements 108 and 210; to cause exposure or saturation to the entirety of the first indicating element 108 to occur simultaneously; to cause exposure or saturation to the entirety of the second indicating element 210 to occur simultaneously; and/or to cause exposure or saturation of both of the first and second indicating elements 108 and 210 simultaneously, or in a controlled progression from distal to proximal ends, to the greatest extent possible. Thus, the structure and configuration of diffuser 120, and its relationship to housing 110, may provide these and other functions in various embodiments.

The diffuser 120 has a generally longitudinal body comprising, in series starting at the proximal end 146, a connection tube section 132, a reference indicator retaining collar 130, a first transition section 182, indicating element section 168, a second transition section 180, a tip section 162, and a distal end 148. The connection tube section 132 forms the proximal end of diffuser fluid passage 115. Fluid passage 115 continues toward the distal end of diffuser 120 as a generally tubular structure interior of and through the collar 130 and the first transition section 182 into the volume contained by the housing 110. Thus, fluid passage 115 couples the port 104, 114 to the housing fluid passage 138.

The indicating element section 168 of the diffuser 120 preferably has one or more fluid-channel-forming structures 140 to support the indicating elements 108 and 210 and to distribute the fluid entering the indicator 100 and housing 110 at the distal end 102, 112 and port 103, 113 thereof to the indicating elements 108 and 210. The fluid-channel-forming structures 140 form one or more fluid channels 154 which are operatively coupled to fluid passage 115.

The second transition section 180 (FIG. 1E) guides fluid from the portion of housing fluid passage 138 in the area of diffuser tip section 162 into the diffuser fluid channels 154.

In operation, the vacuum or suction coupled via port 104, 114 draws fluid via the housing fluid passage 138 into fluid channels 154 and into contact with or in the vicinity of the indicating elements 108 and 210. Under influence of the suction or vacuum, the fluid continues through first transition section 182 and connection tube section 132 and exits to the suction or vacuum system or reservoir. In an embodiment where one or more of indicating elements 108 and 210 are tubes or similar structures surrounding the fluid-channel-forming structure 140 of indicating element section 168, the fluid must travel through the diffuser fluid channels 154 to reach the exit, and accordingly, the fluid must come into contact with or in the vicinity of such indicating elements. If one or more of the indicating elements is generally pervious to the fluid, the fluid may pass through such indicating element. In an initial stage of operation, the fluid transport source and the indicator 100 may be filled with air, which, during continued operation, is eventually replaced by the fluid under test.

In an embodiment 120 of a diffuser constructed according to one aspect of the invention, the indicating element section 168 may have a generally star-shaped cross-section formed by spines, ridges, or vanes 140 extending radially from a central core along the length of indicating element section 168. The vanes 140 further extend longitudinally between the indicating element section 168 to the tip section 162 through the second transition section 180, tapering in diameter. The configuration of vanes 140 form a plurality of substantially independent diffuser fluid channels 154, which may be used to independently expose different indicating elements 108 and 210 or different portions thereof. The plurality of independent diffuser fluid channels 154 could also be used to route fluid from different respective fluid sources to one or more indicating elements; a modification of the housing would be needed to receive fluid from plural sources and separately convey the fluid to respective fluid channels. Other diffuser configurations may also be used, as discussed further.

The indicating elements 108 and 210 are preferably disposed adjacent to the fluid-channel-forming structure 140 of indicating element section 168, and the diffuser fluid channels 154 formed thereby. For example, one or more of indicating elements 108 and 210 may be implemented as a tubular component arranged around the fluid-channel-forming structure 140. One or more of the indicating elements may be constructed as a tube, but could also be a sheet wrapped around the fluid-channel-forming structure 140. The indicating elements 108 and 210 could take other suitable forms, such as strips, ribbons, dots, bars, or the like, and in those forms are preferably disposed adjacent the fluid-channel-forming structure 140 and the diffuser fluid channels 154 formed thereby.

A reference indicator 116 is preferably provided on the diffuser 120 adjacent collar 130. The reference indicator 116 may be constructed as a generally tubular element disposed coaxially around the body of diffuser 120, overlapping the first transition section 182. In the assembled configuration of indicator 100, the reference indicator 116 may coaxially overlap the first section 156 of housing 110 and is preferably adjacent to the indicator window 118 of the housing, so that the reference indicator 116 may be directly compared with one of more of the indicating elements 108 and 210. The reference indicator 116 preferably bears suitable indicia which may be compared with indications displayed by one or more of the indicating elements 108 and 210 to represent a measured value or state of a characteristic or property of the fluid under test.

For example, in an embodiment where the first indicating element 108 is a pH-indicating paper that indicates the measured pH by a corresponding color, the reference indicator 116 may contain a plurality of labeled color samples or indicia, such as 124, 126, and 128, against which the indicating element 108 may be compared by a user to obtain a pH measurement. Although three color samples are depicted in the figures, it will be appreciated that any number of color samples or indicia may be provided to accommodate the range of indications of which the indicating element 108 is capable and to accommodate the desired precision of measurement.

Similarly, the reference indicator 116 may contain one or more labeled color samples or indicia (not shown) that correspond to the appearance of the second indicating element 210 in the defined states to inform a user or observer how to interpret an indication from the second indicating element 210. For example, in an embodiment where the second indicating element 210 is an indicating paper that indicates prior exposure to water or another liquid, the reference indicator 116 may contain one or more labeled color samples or indicia that correspond to the appearance of the second indicating element 210 in the exposed and unexposed states.

Additional reference indicators may be provided for each of first and second indicating elements 108 and 210, and for plural instances of the indicating elements 108 and 210. Plural reference indicators may be provided to facilitate comparison between an indicating element and a corresponding, juxtaposed or proximate, reference indicator. Where multiple instances of the indicating elements are adapted to sense the same properties with the same indications, such as where the same type of indicating medium is placed in multiple locations in an indicator device, the same reference indicator may be used for such instances.

The reference indicator 116 may contain other indicia as appropriate for the types of indicating elements 108 and 210 being used and the fluid characteristic or property (or a plurality thereof) to which the indicating elements 108 and 210 are sensitive. For example, in some embodiments, the one or more of first and second indicating elements 108 and 210 could distinguish fluid characteristics or properties by displaying shapes, letters, icons, or other symbols instead of colors, and the reference indicator 116 could present reference examples of the symbols for comparison. Reference indicator 116 may also provide a legend or other indicia which may, for example, be used to interpret the symbols or other indicia displayed by indicating elements 108 and 210.

While is it usually desirable that the entirety of indicating elements 108 and 210 be uniformly saturated with the fluid under test, characteristics of the fluid, or limited availability of the fluid, could result in only a limited extent of the indicating elements 108 and 210 being exposed or providing a useful indication of the measured fluid characteristic or property. Also, in some embodiments, one of more of indicating elements 108 and 210 may not be present or visible throughout all angles about the general longitudinal axis of indicator 100. Further, in some embodiments, plural indicating elements (or plural portions or segments of a single indicating element) sensitive to different characteristics or properties may be visible at respective properties about the axis. Reference indicator 116 is preferably rotatable so that any of its samples or indicia may be brought into juxtaposition with a visible portion of a corresponding indicating element, such as 108 or 210, through indicator window 118 to enable comparison of the indication with the reference sample or indicia.

In an embodiment in which indicator 100 is constructed by assembling the diffuser 120 telescopically into the proximal-end opening 152 of the housing 110, it is preferable that in the assembled configuration the diffuser 120 occupy desired longitudinal and angular positional relationships with respect to the housing 110. Housing 110 preferably has one or more locator structures 164 which help ensure that the desired positional relationships will be achieved in the assembly process. The locator structures 164 may be formed as tabs or other sections of reduced inside diameter of the housing in the area of the proximal-end opening 152 of the housing 110. The locator structures 164 cooperate with locator structures on the diffuser. (See, for example, locator structures 320 of FIG. 3F.) For example, the locator structures 164 may be formed as tabs or other sections of increased outside diameter on a telescoping segment of the diffuser 120, having a shape or profile that compliments or mates with the shape of locator structures 164. In one embodiment constructed according to an aspect of the invention, the locator structures on at least one of the housing 110 and the diffuser 120 provide a substantially triangular socket for receiving the mating structure of the other component, as best seen in FIG. 1E. The use of the triangular mating shapes allows the components to self-align, with respect to their angular position, during telescopic assembly. This reduces or eliminates the need to begin assembly with the components in a specific angular alignment. The locator structures 164 and, and complementary locator structures on the diffuser, preferably have respective interference faces which, upon completion of the telescopic assembly, oppose one another and are in secure contact with one another. For example, locator structures 164 of housing 110 have interference faces 166. The locator structures and interference faces thereof of diffuser 120 are not visible in FIGS. 1A-1E, but similar locator structures 320 and locator structure interference faces 324 are provided on diffuser 310 (see FIG. 3F).

In embodiments in which the housing 110 and diffuser 120 are assembled to form the indicator 100, in addition to its other functions, the diffuser 120 or a portion thereof may function as a closure or end cap for the housing 110, and therefore, it is desirable that a seal be formed between the housing 110 and the diffuser 120. The seal could be formed at any interfaces of these components. Where a seal depends on contact between mating or abutting surfaces, the contact may be a tight, friction or interference contact to achieve an effective seal. In an embodiment constructed according to an aspect of the invention, the seal may be formed where a collar of the diffuser 120 is in contact with the surface of the proximal end 150 of the housing. The seal may also be formed where the outer diameter of the diffuser body 120 (see, for example, outside sealing surface 344 of FIG. 3F) is in contact with the inner diameter of the housing 110. One or both of these interfacing components may have a taper or other shape which enhances the interference fit of these components, enhancing the seal provided thereby. Further, the seal may be formed where the locator structure interference faces 166 of the housing 110 are in contact with the complementary locator structure interference faces (e.g., faces 324, FIG. 3F) of the diffuser 120. The aggregate length and surface area along which these complementary faces are in contact can enhance the effectiveness of the seal. Moreover, several or all of these sealing interfaces may cooperate or contribute to an effective seal of the housing 110 and the diffuser 120. Where a seal depends on contact between mating or abutting surfaces, the contact may be a tight, friction or interference contact to achieve an effective seal. In addition, a seal may also be achieved using a gasket, sealant, adhesive, chemical weld, thermal weld, ultrasonic weld, or the like, which may be applied or performed at or near the sealing interfaces.

FIG. 3F is a side isometric view of an alternative example embodiment of a diffuser 310 which may be used in constructing an indicator 100 in accord with a further aspect of the invention. Diffuser 310 is generally similar to the diffuser 120 of FIGS. 1C, 1F, and 1G as heretofore described, and except for the differences explained below, the foregoing description of diffuser 120 is applicable to diffuser 310, mutatis mutandis, and is hereby incorporated by reference. Diffuser 310 has a body generally similar to that of diffuser 120. Diffuser 310 has a proximal end 312 and a distal end 314. A connection tube 316 extends from a port 336 at the proximal end 312 and forms a fluid communication chamber, lumen, or fluid passage 338. FIG. 3G is an end view of the diffuser 310 taken from the distal end 314. FIG. 3H is an end view of the diffuser 310 taken from the proximal end 312. In combination, FIGS. 3F, 3G, and 3H depict some features common to diffuser 120 and diffuser 310 that are not visible in FIGS. 1A-1E, and accordingly, the description of those features here is also applicable to diffuser 120 of FIGS. 1A-1E.

Diffuser 310 has a first indicating element section 346, which is attached or connected to the distal extension of connection tube 316 by a channel-forming support 326. A transition section 348 having a tapered diameter provides a transition between the distal end of the first indicating element section 346 and a tip section 334 at the distal end of the diffuser body. Between the legs of the support 326, openings or channels 328 allow fluid communication between fluid passage 338 and the volume to the distal side of the connection tube 316. Thus, openings 328 and connection tube 316 provide a fluid passage 338 between the proximal end port 336 and the remaining volume enclosed by the housing (not shown), which corresponds to the fluid passage thereof. Channel-forming support 326 allows flow through the center of the device. The four chambers at the base allow air and fluid flow during suction and pressure. The four openings 328 allow fluid and air to flow directly in-line with the diffuser to maximize vacuum pressure and fluid flow.

Proximal end 312 preferably has an appropriate fitting 340 for a secure and sealed coupling to a source of vacuum or suction. For example, fitting 340 may be formed as male Luer lock attachment threads on the outer wall of connection tube 316. Any other appropriate coupling or fitting could also be used.

A collar 318 disposed at an intermediate position along connection tube 316 has a diameter larger than that of the connection tube 316 and approximately that of the housing. The collar 318 provides a distal surface 342 against which the proximal end of the housing can bear. In some embodiments, the distal surface 342 and the proximal end of the housing may cooperate to achieve a seal between the housing and the diffuser 310. The collar also serves to retain the reference indicator (not shown in FIGS. 3F and 3H; see reference indicator 116 of FIGS. 1A, 1D, and 1E).

The portion of the connection tube 316 extending distally from the collar 318 has an outside surface 344. When the housing and diffuser 310 are in the assembled configuration, the outside surface 344 and the inside diameter of the housing may be sized, to achieve an interference fit, and may form a seal between the housing and the diffuser 3 10. The outside surface 344 may also have a taper to enhance its interference fit with the inside diameter of the housing. One or more locator structures 320 are preferably provided on the outside surface 344 of the distal extension of connection tube 316. The locator structure 320 may be formed as structures of increased diameter which cooperate with complementary locator structures on the inner surface near the proximal end of the housing to assist in ensuring that, upon assembly, the diffuser 310 and the occupy desired longitudinal and angular positional relationships with respect to one another.

As best seen in FIG. 3F, as part of the locator structure 320, diffuser 310 has a substantially triangular socket 322 for receiving a complementary part of the locator structure of the housing (see locator structure 164, FIG. 1E). The triangular shape of the socket 322 and complementary part on the associated housing (e.g., housing 110) provide a self-aligning capability, such that it is not necessary that the parts be angularly aligned (about the longitudinal axis) prior to assembly. When a diffuser 310 and a housing are assembled as an indicator, the locator structure interference faces 324 are in contact with the complementary locator structure interference faces 166 (FIG. 1E) of the housing. These contacting faces 324 and 166 may contribute to the seal between the diffuser and the housing. The aggregate length and surface area along which these complementary faces are in contact can enhance the effectiveness of the seal.

The first indicating element section 346 has a fluid-channel-forming structure 330 which forms one or more fluid channels 332 and may support one or more indicating elements (not shown in FIG. 3F, but similar indicating elements 108 and 210 are shown in FIGS. 1A, 1D, and 1E) in close proximity thereto. In operation, vacuum or suction coupled via the proximal end port 336 draws fluid into the housing. As best seen in FIG. 3F, the fluid-channel-forming structure 330 and fluid channels 332 have a generally spiral shape in the area of transition section 348 and a generally helical shape in the area of the first indicating element section 346. The fluid-channel-forming structure 330 of diffuser 310 has six turns in the depicted embodiment. A different number of turns may be preferred depending on characteristics of the fluid to be tested, such as viscosity and the presence of solids. The presence of an indicating element or the inner wall of the housing substantially prevents the fluid from exiting except through fluid channels 332 and then through openings 328 into connection tube 316. As a result all or nearly all of the fluid flows though fluid channels 332, in contact with or in close proximity to the indicating element. The shape, limited depth, and configuration of the fluid channels 332 force the fluid in the radial-outward direction. This helps distribute the fluid so as to cause nearly all of the area of the first indicating element to be exposed to the fluid as it passes through the fluid channels 332, and this favorable distribution of fluid can occur regardless of the position or orientation of the indicator. This fluid behavior is characteristic of each of the diffusers having spiral or helical fluid channels described herein. If the first indicating element is generally pervious to the fluid, the fluid may pass through the indicating element.

Diffuser 310 has a second indicating element section 246 located near the distal end of the distal extension of connection tube 316. The second indicating element section may 246 be defined by a region 252 of slightly reduced diameter of the distal extension of the connection tube 316, between the distal end of the extension and the distal ends of locator structure 320, which may be realized as locator tabs. This reduced diameter provides a location for a second indicating element 248 to reside—i.e., between the outside surface 344 of the distal extension of the connection tube 316 and the inner surface of a housing (not shown) into which the diffuser telescopically nests in an assembled indicator that uses the diffuser 310. One or more openings 250 may be provided in the wall of the distal extension of the connection tube 316 leading to the interior fluid communication passage 338. The openings 250, if provided, may allow fluid to flow along or through the second indicating element 248 from the first indicating element section 346 into the interior fluid communication passage 338. Some indicating media which may be used to implement the second indicating element 248 may allow fluid flow through the medium. For example, the second indicating element 248 may be constructed from felt, sponge, a woven, knitted, or non-woven fabric, or other porous, fibrous, or generally pervious material through which fluid may flow or seep. Other types of indicating media may adequately absorb fluid from an edge or surface and so may not require openings such as openings 250.

The reference symbol “Y1” is used in the Figures generally to denote one or more instances of the second indicating element where the second indicating element is located at the distal end of a distal extension of a connection tube of the diffuser and is located further from the source of fluid being tested than the first indicating element. The configuration of first and second indicating elements described in connection with FIG. 3F corresponds to the first schematic arrangement 5010 of FIG. 50.

In some cases, fluid or air flow through the device, or handling of the device, may tend to dislodge the indicating elements 108, 210 from preferred locations with respect to the indicator window 118 or other elements. It is generally desirable that the indicating elements not move when the indicator is in use. The fluid-channel-forming structure 330 may have one or more radially-extending tabs 352 to assist in retaining the indicating element in a preferred location. The indicating element retaining tabs 352 may be used on any of the diffusers described herein. The indicating elements could also be secured or fixed in location with respect to the diffuser using other structures such as clips or burrs, adhesive attachment, other chemical attachment means, thermal, chemical, or ultrasonic welding, or any other appropriate securement means. Any such indicating element retaining tabs or other means may be used with any of the diffusers disclosed herein.

As best seen in FIGS. 3G and 3H, collar 318 may have a flat section 350 along a portion of the outer surface, instead of being perfectly round, to provide a key or index that may be used to align the diffuser during assembly or other manufacturing steps. A key or index may also be provided on the housing 1310 to align it during assembly or manufacturing. The flat section 350 may be used in conjunction with other locator structures (e.g., locator structures of FIGS. 3F and 3G) or without these locator structures.

FIGS. 3A, 3B, 3C, and 3D, are side isometric views of several alternative example embodiments of diffusers which may be used in constructing an indicator 100 in accord with further aspects of the invention. Any of these example embodiments may be used in the indicator 100 in place of the diffuser 120 hereinbefore described.

FIG. 3A is a side isometric view of an embodiment 360 of a diffuser constructed according to a further aspect of the invention. Diffuser 360 is generally similar to the diffuser 120 of FIGS. 1A-1E and the diffuser 310 of FIGS. 3F and 3H, as heretofore described, and except for the differences explained below, the foregoing descriptions of diffusers 120 and 310 are applicable to diffuser 360, mutatis mutandis, and are hereby incorporated by reference.

Diffuser 360 has a body generally similar to that of diffuser 310. A spiral fluid-channel-forming structure 362 forms a fluid channel 364, similar to fluid-channel-forming structure 330 and fluid channels 332 of diffuser 310. The fluid-channel-forming structure 362 and fluid channel 364 have three turns instead of six. A conical transition region 366 joins the fluid-channel-forming structure 362 to a tip section 368, which is shorter than the tip section of diffuser 310. Locator structures 369, similar in function to the locator structures 320 of diffuser 310, are formed as generally rectangular tabs with trapezoidal enlargements near their proximal ends 367 adjacent collar 361. A corresponding modification to a housing would be required to accommodate the difference in the shape of the diffuser from that of the diffusers 120 and 310.

FIG. 3B is a side isometric view of an embodiment 370 of a diffuser constructed according to a further aspect of the invention. Diffuser 370 is generally similar to the diffuser 120 of FIGS. 1A-1E and the diffuser 310 of FIGS. 3F and 3H, as heretofore described, and except for the differences explained below, the foregoing descriptions of diffusers 120 and 310 are applicable to diffuser 370, mutatis mutandis, and are hereby incorporated by reference.

Diffuser 370 has a body generally similar to that of diffuser 310. A spiral fluid-channel-forming structure 372 forms a fluid channel 374, similar to fluid-channel-forming structure 330 and fluid channels 332 of diffuser 310. The fluid-channel-forming structure 372 and fluid channel 374 have six turns. A conical transition region 376 joins the fluid-channel-forming structure 372 to a tip section 378, which is shorter than the tip section of diffuser 310. Locator structures 379, similar in function to the locator structures 320 of diffuser 310, are formed as generally rectangular tabs with trapezoidal enlargements near their proximal ends 377 adjacent collar 371. A corresponding modification to a housing would be required to accommodate the difference in the shape of the diffuser from that of the diffusers 120 and 310. A flat section 373 on the outer surface of collar 371 provides a key or index that may be used to align the diffuser 370 during assembly or other manufacturing steps.

FIG. 3C is a side isometric view of an embodiment 380 of a diffuser constructed according to a further aspect of the invention. Diffuser 380 is generally similar to the diffuser 120 of FIGS. 1A-1E and the diffuser 310 of FIGS. 3F and 3H, as heretofore described, and except for the differences explained below, the foregoing descriptions of diffusers 120 and 310 are applicable to diffuser 380, mutatis mutandis, and are hereby incorporated by reference.

Diffuser 380 has a body generally similar to that of diffuser 310. A spiral fluid-channel-forming structure 382 forms a fluid channel 384, similar to fluid-channel-forming structure 330 and fluid channels 332 of diffuser 310. The fluid-channel-forming structure 382 and fluid channel 384 have nine turns instead of six. A spiral transition region 386 joins the fluid-channel-forming structure 382 to a tip section 388 which is similar to the tip section 334 of diffuser 310. Locator structures 389, similar in function to the locator structures 320 of diffuser 310, are formed as generally rectangular tabs with trapezoidal enlargements near their proximal ends 387 adjacent collar 381.

FIG. 3D is a side isometric view of an embodiment 390 of a diffuser constructed according to a further aspect of the invention, including a further example embodiment of a second indicating element which may be used with any of the diffusers disclosed herein. FIG. 3E is a partial cutaway view of the diffuser 390 taken along the view lines 3E-3E of FIG. 3D. Diffuser 390 is generally similar to the diffuser 120 of FIGS. 1A-1E and the diffuser 310 of FIGS. 3F and 3H, as heretofore described, and except for the differences explained below, the foregoing descriptions of diffusers 120 and 310 are applicable to diffuser 390, mutatis mutandis, and are hereby incorporated by reference.

Diffuser 390 has a body generally similar to that of diffuser 310. A star, or a cross-shaped fluid-channel-forming structure 392, forms a fluid channel 394 that is similar to fluid-channel-forming structure 140 and fluid channels 154 of diffuser 120. A tapered transition region 396 joins the fluid-channel-forming structure 392 to a tip section 398, which is shorter than the tip section of diffuser 120. Locator structures 399, similar in function to the locator structures 320 of diffuser 310, are formed as generally rectangular tabs with trapezoidal enlargements near their proximal ends 397 adjacent collar 391. A corresponding modification to a housing would be required to accommodate the difference in the shape of the diffuser from that of the diffusers 120 and 310. A flat section 393 on the outer surface of collar 391 provides a key or index that may be used to align the diffuser 390 during assembly or other manufacturing steps.

Diffuser 390 has a first indicating element section 260, in the area of the fluid-channel-forming structure 392, where a first indicating element (not shown) resides. Diffuser 390 has a second indicating element section 262, where a second indicating element 264 resides. The second indicating element 264 is located near the distal end 395 of a distal extension of the connection tube 266 of the diffuser 390, and may, for example, be formed as a cylindrical or tubular element and located in the region between the locator structures 399 and the extreme distal end of the extension. The second indicating element 264 may be constructed of indicator media of any of the types and characteristics described earlier in connection with the second indicating elements, e.g., elements 210 of FIGS. 1A, 1D, and 1E, and 5020 of FIG. 50. The diameter of the distal extension of the connection tube 266 in the area of the second indicating element section 262 may be reduced to accommodate the thickness of the second indicating element 264. The configuration of first and second indicating elements described in connection with FIG. 3D corresponds to the first schematic arrangement 5010 of FIG. 50.

FIG. 3D2 is a side isometric view of a further embodiment 290 of a diffuser constructed according to an aspect of the invention, including a further example embodiment of a second indicating element which may be used with any of the diffusers disclosed herein. FIG. 3E2 is a partial cutaway view of the diffuser 290 taken along the view lines 3E2-3E2 of FIG. 3D2. Diffuser 290 is generally similar to the diffuser 390 of FIGS. 3D and 3E, diffuser 120 of FIGS. 1A-1E, and diffuser 310 of FIGS. 3F and 3H, as heretofore described, and except for the differences explained below, the foregoing descriptions of diffusers 120, 310, 390 are applicable to diffuser 290, mutatis mutandis, and are hereby incorporated by reference.

Diffuser 290 has a body generally similar to that of diffuser 310. A star, or a cross-shaped fluid-channel-forming structure 292, forms a fluid channel 294, similar to fluid-channel-forming structure 140 and fluid channels 154 of diffuser 120. A tapered transition region 296 joins the fluid-channel-forming structure 292 to a tip section 298, which is shorter than the tip section of diffuser 120. Locator structures 299, similar in function to the locator structures 320 of diffuser 310, are formed as generally rectangular tabs with trapezoidal enlargements near their proximal ends 297 adjacent collar 291. A corresponding modification to a housing would be required to accommodate the difference in the shape of the diffuser from that of the diffusers 120 and 310. A flat section 293 on the outer surface of collar 291 provides a key or index that may be used to align the diffuser 290 during assembly or other manufacturing steps.

Diffuser 290 has a first indicating element section 270, in the area of the fluid-channel-forming structure 292, where a first indicating element (not shown) resides. Diffuser 290 has a second indicating element section 272, where a second indicating element 274 resides. The second indicating element 274 is located along a distal extension of the connection tube 276 of the diffuser, and may, for example, be formed as one or more segments on the outer surface of the extension in the region of the locator structures 299 between the distal side of collar 291 and the distal end 295 of the extension of the connection tube. The second indicating element 274 may be constructed of indicator media of any of the types and characteristics described earlier in connection with the second indicating elements, e.g., elements 210 of FIGS. 1A, 1D, and 1E, and 5020 of FIG. 50. The diameter of the distal extension of the connection tube 276 in the area of the second indicating element section 272 may be reduced to accommodate the thickness of the second indicating element 274. It is desirable to provide surfaces that allow a seal between the housing and the diffuser when the diffuser 390 is nest ably assembled with a housing (not shown). If the second indicating element 274 extends only part way to the collar 291, a seal may be formed between the inner surface of the housing and the outer surface of the extension tube. In addition or as an alternative, the proximal end of the housing may seal against the distal end of collar 291.

The reference symbol “Y2” is used in the Figures generally to denote one or more instances of the second indicating element where the second indicating element is located on the outer surface a distal extension of a connection tube of the diffuser in the area of its locator structures and is located further from the source of fluid being tested than the first indicating element. The configuration of first and second indicating elements described in connection with FIG. 3D2 corresponds to the first schematic arrangement 5010 of FIG. 50.

FIGS. 4A, 4B, 5A, 5B, 5C, 5E, 6A, 6B, 6C, 6D, 6F, and 6G depict several example embodiments, according to further aspects of the invention, of apparatus for obtaining and containing a fluid obtained from a person or animal, measuring or detecting one or more characteristics of the fluid, and displaying an indication relating to the measurement. The embodiments of FIGS. 4A, 4B, 5A, 5B, 5C, 5E, 6A, 6B, 6C, 6D, 6F, and 6G, the “low-volume embodiments”, are generally similar to the earlier described embodiments of 1A-1E, 2A-2F, and 3A-3H, but encompass modifications from those earlier described that render the embodiments more suitable for use when a very limited volume of fluid is available to be tested. These latter embodiments may be appropriate for use with neonatal patients, veterinary patients, and other similar instances in which the patient is small or limited fluid is available to be tested.

In the discussion of the low-volume embodiments below, newly-introduced example embodiments may be described with reference to similarity with an earlier-described example embodiment. In each case, the foregoing description of the corresponding referenced embodiment is hereby incorporated by reference, and the newly-introduced embodiment shall be considered to have structures, features, components, and functions similar to or equivalent to the referenced embodiment, mutatis mutandis, except for: (i) items specifically identified as different in the description; and (ii) the following general low-volume modification. Each of the newly-introduced embodiments is modified from the referenced embodiment in that its size is reduced, and the volumes of fluid-transfer passages, conduits, chambers, lumina, and other interstitial spaces which fluid under test may occupy, are reduced or minimized. In addition to reducing the volume of fluid needed to accomplish a measurement, this modification also reduces both the amount of distance the fluid must travel to reach and saturate the indicating elements, and the elapsed time of such travel (compared to those required in connection with the earlier-described indicators).

FIG. 4A is an isometric view of an embodiment of an indicator device 410 constructed according to an aspect of the invention, which is generally similar to the indicator 100 of FIGS. 1A-1E, subject to the low-volume modification. Indicator 410 comprises a housing 420 and a diffuser 430, which are respectively similar to housing 110 and diffuser 120 of FIGS. 1A-1E, subject to the low-volume modification. The indicator 410 includes a proximal end 427 and a distal end 426. FIG. 4B is an end view of the indicator 410 of FIG. 4A, looking toward the proximal end 427 thereof; FIG. 4C is a side isometric view of the housing 420. A flat section 424 on the outer surfaces of both housing 420 and diffuser 430 provides one or more keys or indices that may be used to align the housing 420 and diffuser 430 during assembly or other manufacturing steps. The housing 420 has a main body section 428 and a thin distal tip 426 for receiving fluid from a fluid transport source (not shown), which may take the form of tubing, conduits, adapters, appliances, containers, reservoirs, catheters, feeding tubes, suction tubes, or the like, which may be used in a clinical environment to furnish the fluid with which indicator 410 is to be used to perform a test, or obtain a measurement, or indication of one or more characteristic or property of the fluid. The distal end 426 may have a diameter and be formed with a “catheter tip” shape suitable for mating, coupling, or connection with a catheter, such as a nasogastric tube or feeding tube which is inserted into a patient's stomach and which serves as the fluid transport source. Further, the size and shape of distal end 426 may be selected to be suitable for use with the types of tubes, catheters, or other fluid transport source that are used to treat neo-natal or other very small patients. The catheter tip can also be mated with other types of receiving connector, plugs, hubs, and the like. A distal transition section 422 provides the transition between the main body section 428 and the distal end 426. The shape of the distal transition section 422 differs from the shape of the housings 110 and 190 of FIGS. 1A-1E, 2B, 2C, and 2F in that the distal transition section 422 has a snubbed, relatively sharp taper, instead of an elongated gradual taper. The shape of distal transition section 422 is selected to minimize the internal volume so as to maximize the amount of available fluid that is routed to the indicating elements. In addition to reducing the volume of fluid needed to accomplish a measurement, this feature also reduces both the amount of distance the fluid must travel to reach and saturate the indicating elements, and the elapsed time of such travel (compared to those required in connection with the earlier-described indicators).

FIG. 5A is view of a further example embodiment of a housing 440, constructed according to an aspect of the present invention, of a housing which is similar to the housing 420 of FIGS. 4A-4C, but which provides a central fluid passage modified cross section to incorporate fluid guide channels 442 on the interior surface 444 of distal transition section 448. Housing 440 is further similar to housing 190 of FIGS. 2B-2F, subject to the low-volume modification. FIG. 5B is a cutaway view of the housing 440 of FIG. 5A taken along the view lines 5B-5B. FIG. 5C is a partial cutaway view of the housing 440 of FIGS. 5A-5B. FIG. 5D is an end view looking into the housing 440 of FIGS. 5A-5C from the proximal end thereof. FIG. 5E is an end view of the housing 440 of FIGS. 5A-5D looking toward the distal end 446 thereof.

FIG. 6F is a view of a further example embodiment 610, constructed according to an aspect of the present invention, of a diffuser which is similar to the diffuser 310 of FIGS. 3F-3H, subject to the low-volume modification, and including a further example embodiment of a second indicating element which may be used with any of the diffusers disclosed herein. FIG. 6G is a distal end view of the diffuser 610. FIG. 6H is a proximal end view of the diffuser 610. Diffuser 610 has a fluid-channel-forming structure 612 and a fluid channel 616 formed thereby. The proximal end is shown by 619. The fluid-channel-forming structure 612 and fluid channel 616 are in a spiral configuration and have five turns. Diffuser 610 further has a sharp-tapered transition section 614 and a shorter distal tip 618, which accommodates a smaller housing that more efficiently routes available fluid to the indicating elements.

Diffuser 610 has a first indicating element section 1602, in the area of the fluid-channel-forming structure 612, where a first indicating element (not shown) resides. Diffuser 610 has a second indicating element section 1604, where a second indicating element 1606 resides. The second indicating element 1606 is located near the distal end 1616 of a distal extension 1610 of the connection tube 1608 of the diffuser, and may, for example, be formed as a cylindrical or tubular element and located in the region between locator structures 1612 and the distal end 1616 of the distal extension 1610. The second indicating element 1606 may be constructed of indicator media of any of the types and characteristics described earlier in connection with the second indicating elements, e.g., elements 210 of FIGS. 1A, 1D, and 1E, 5020 of FIG. 50. The diameter of the distal extension 1610 of the connection tube 1608 in the area of the second indicating element section 1604 may be reduced to accommodate the thickness of the second indicating element 1606. The configuration of first and second indicating elements described in connection with FIG. 6F corresponds to the first schematic arrangement 5010 of FIG. 50.

FIG. 6A is a side view of a further example embodiment 630, constructed according to an aspect of the present invention, of a diffuser which is similar to the diffuser 360 of FIG. 3A, exhibiting a modification to accommodate lower fluid volume than earlier-described embodiments. Diffuser 630 has a fluid-channel-forming structure 632 and a fluid channel 636 formed thereby. The fluid-channel-forming structure 632 and fluid channel 636 are in a spiral configuration and have two turns. Diffuser 630 further has a sharp-tapered transition section 634 and a shorter distal tip 638, which accommodates a smaller housing that more efficiently routes available fluid to the indicating elements.

FIG. 6B is a view of a further example embodiment 640, constructed according to an aspect of the present invention, of a diffuser which is similar to the diffuser 360 of FIG. 3A, exhibiting a lower-fluid-volume modification. Diffuser 640 has a fluid-channel-forming structure 642 and a fluid channel 646 formed thereby. The fluid-channel-forming structure 642 and fluid channel 646 are in a spiral configuration and have four turns. Diffuser 640 further has a sharp-tapered transition section 644 and a shorter distal tip 648, which accommodates a smaller housing that more efficiently routes available fluid to the indicating elements.

FIG. 6C is a view of a further example embodiment 650, constructed according to an aspect of the present invention, of a diffuser which is similar to the diffuser 360 of FIG. 3A, exhibiting a lower-fluid-volume modification. Diffuser 650 has a fluid-channel-forming structure 652 and a fluid channel 656 formed thereby. The fluid-channel-forming structure 652 and fluid channel 656 are in a spiral configuration and have five turns. Diffuser 650 further has a sharp-tapered transition section 654 and a shorter distal tip 658, which accommodates a smaller housing that more efficiently routes available fluid to the indicating elements.

FIG. 6D is a view of a further embodiment 660, constructed according to the present invention, of a diffuser which is similar to the diffuser 390 of FIG. 3D, exhibiting a lower-fluid-volume modification, and including a further example embodiment of a second indicating element which may be used with any of the diffusers disclosed herein. FIG. 61 is an end view of the diffuser 660 of FIG. 6D, taken along the view lines 61-61 thereof, looking toward the distal end 668 thereof. Diffuser 660 has a fluid-channel-forming structure 662 and fluid channels 666 formed thereby. The fluid-channel-forming structure 662 has a star- or cross-shaped cross-section forming longitudinally-extending parallel fluid channels 666. Diffuser 660 further has a sharp-tapered transition section 664 and a shorter distal tip 668, which accommodates a smaller housing that more efficiently routes available fluid to the indicating elements.

Diffuser 660 has a first indicating element section 1622, in the area of the fluid-channel-forming structure 662, where a first indicating element (not shown) resides. Diffuser 660 has a second indicating element section 1624, where a second indicating element 1626 resides. The second indicating element 1626 is located near the distal end 1628 of a distal extension 1630 of a connection tube 1632 of the diffuser, and may, for example, be formed as a cylindrical or tubular element and located in the region between the locator structures 399 and the distal end 1628 of the distal extension 1630. The second indicating element 1626 may be constructed of indicator media of any of the types and characteristics described earlier in connection with the second indicating elements, e.g., elements 210 of FIGS. 1A, 1D, and 1E, and 5020 of FIG. 50. The diameter of the distal extension 1630 of the connection tube 1632 in the area of the second indicating element section 1624 may be reduced to accommodate the thickness of the second indicating element 1626. The configuration of first and second indicating elements described in connection with FIG. 6D corresponds to the first schematic arrangement 5010 of FIG. 50.

FIG. 6D2 is a view of a further embodiment depicting the diffuser 1660, constructed according to the present invention, which is similar to the diffuser 660 of FIG. 6d and the diffuser 390 of FIG. 3D, exhibiting a lower-fluid-volume modification, and including a further example embodiment of a second indicating element which may be used with any of the diffusers disclosed herein. FIG. 612 is an end view of the diffuser 1660 of FIG. 6D2, taken along the view lines 612-612 thereof, looking toward the distal end 1668 thereof. Diffuser 1660 has a fluid-channel-forming structure 1662 and fluid channels 1666 formed thereby. The fluid-channel-forming structure 1662 has a star, or a cross-shaped, cross-section forming longitudinally-extending parallel fluid channels 1666. Diffuser 1660 further has a sharp-tapered transition section 1664 and a shorter distal tip 1668, which accommodates a smaller housing that more efficiently routes available fluid to the indicating elements.

Diffuser 1660 has a first indicating element section 1670, in the area of the fluid-channel-forming structure 1662, where a first indicating element (not shown) resides. Diffuser 1660 has a second indicating element section 1684, where a second indicating element 1686 resides. The second indicating element 1686 is located along a distal extension 1690 of the connection tube 1692 of the diffuser, and may, for example, be formed as one or more segments on the outer surface of the distal extension 1690 in the region of the locator structures 1694 between the distal side of collar 1696 and the distal end 1688 of the extension of the connection tube. The second indicating element 1686 may be constructed of indicator media of any of the types and characteristics described earlier in connection with the second indicating elements, e.g., elements 210 of FIGS. 1A, 1D, and 1E, and 5020 of FIG. 50. The diameter of the distal extension 1690 of the connection tube 1692 in the area of the second indicating element section 1684 may be reduced to accommodate the thickness of the second indicating element 1686. It is desirable to provide surfaces that allow a seal between the housing and the diffuser when the diffuser 1660 is nestably assembled with a housing (not shown). If the second indicating element 1686 extends only part way to the collar 1696, a seal may be formed between the inner surface of the housing and the outer surface of the extension tube. In addition or as an alternative, the proximal end of the housing may seal against the distal end of collar 1696. The configuration of first and second indicating elements described in connection with FIG. 6D2 corresponds to the first schematic arrangement 5010 of FIG. 50.

FIG. 1F is a partial exploded view of another example embodiment 500, according to a further aspect of the invention, of apparatus for obtaining and containing a fluid obtained from a person or animal, measuring or detecting one or more characteristics of the fluid, and displaying an indication relating to the measurement. Indicator 500 is generally similar to the indicator 100 of FIGS. 1A-1E, but the functions of controlling/routing the flow of fluid into contact with the indicating elements are provided by plural individual components rather than a single, integrated diffuser element. Except for the differences explained hereinbelow, the foregoing description of indicator 100 is applicable to indicator 500, mutatis mutandis, and is hereby incorporated by reference.

Indicator 500 has a housing 510, a diffuser element 530 contained in the housing, and a proximal end cap 550 that closes and seals the housing. The housing 510 has a generally tubular body 568 with a distal end 512 and a proximal end 518. The distal end 512 has an intake port or opening 514. The proximal end 518 has a proximal end opening 516. A central fluid communication chamber, lumen, or housing fluid passage 566 extends between distal end port 514 and housing proximal end opening 516. The housing body 568 has, adjoined in series from the distal end 512 to the proximal end 518 a tapered main section 522, indicator window section 520, and a race section of slightly reduced diameter to accommodate a rotatable reference indicator or wheel.

A diffuser 530 is disposed within the interior of body 568 in the housing fluid passage 566. The diffuser has a distal end 532, a tip section 536 and a proximal end 534. Between the tip section 536 and the proximal end 534 are one or more fluid-channel-forming structures 538, which, in combination with any central body portion of the diffuser 530, the body 568, and the indicating elements 542 and 242 form one or more fluid channels 540. Although the diffuser 530 of FIG. 1F is depicted as having a star-shaped cross section, but other diffuser geometries could also be used. For example, the geometries of the fluid-channel-forming structures of any of diffusers 330, 360, 370, 380, 390 could be used, with appropriate modifications to accommodate the differences in the shape of the housing and the connection to proximal end cap 550.

A first indicating element 542 may be formed as a tube or roll and be disposed coaxially with the diffuser. A second indicating element 242 may also be formed as a tube or roll and be disposed coaxially with the diffuser at a location further from the source of fluid being tested than the first indicating element. The first and second indicating elements 542 and 242 may be implemented as two separate objects or indicating media, but could also be sections of a single object or indicating medium. The first and second indicating elements 542 and 242 may be constructed of indicator media of any of the types and characteristics described earlier in connection with the first and second indicating elements, e.g., elements 108 and 210 of FIGS. 1A, 1D, and 1E, 5016 and 5020 of FIG. 50. This configuration of first and second indicating elements corresponds to the first schematic arrangement 5010 of FIG. 50.

The reference indicator 546 is preferably disposed coaxially around the body 568 at the reference indicator race 564. The reference indicator 546 preferably has an inside diameter smaller than the outside diameter of the body 568 at the indicator window section 520 but larger than the diameter of the reference indicator race 564, so that the reference indicator 546 may be retained between the indicator window section 520 and a collar 552 of the proximal end cap 550. Preferably, the reference indicator 546 may be rotated about a longitudinal axis of the indicator 500 by the user.

The proximal end cap 550 acts as a closure and seal for the housing 510 and provides a proximal end or outflow port 554 in fluid communication with the housing fluid passage 566. The proximal end cap 550 has a connection tube 558 extending from proximal end port 554 through collar 552. The connection tube 558 has a portion extending distally of the collar 552. The closure and seal may be achieved by a tight friction or interference fit between the housing inner wall surface 562 and the outer surface of the connection tube extension 560. The seal may also be achieved using a gasket, sealant, adhesive, chemical weld, thermal weld, ultrasonic weld, or the like, which may be applied or performed at or near the sealing interfaces. A Luer lock fitting 556 may be provided on the proximal end port 554 to facilitate connection to a source of vacuum or suction.

A port 570 may be provided on a portion of the housing 510 to allow fluid to be withdrawn, e.g., for other testing, examination, treatment, diagnosis, or procedures. The port 570 also allows reagents, dies, other chemicals, or other material to be added. This could be used, for example, where the indicator may furnish a measurement only with the added material, or where the indicator may furnish an additional useful measurement, or additional precision in measurement, after the added material. The port 570 may be by a puncturable seal, so that material may be added or removed via a needle and syringe or the like, and the seal may be a self-reclosing seal to avoid leakage in or out through the port after use.

The indicator 500 may operate in the same way as indicator 100 of FIGS. 1A-1E.

FIG. 7A is a side view of an adapter 700 constructed according to an aspect of the present invention for converting from a catheter tip connection to a locking or threaded fitting such as a Luer lock fitting. FIG. 7B is a cutaway view of the adapter 700 of FIG. 7A. As best seen in FIG. 7B, the adapter 700 has a proximal end 718 with a female catheter tip socket or receptacle 710 and a proximal end 712 with a female threaded locking fitting 714, such as a Luer lock fitting. A fluid passage 716 allows fluid communication between the receptacle 710 and the fitting 714. FIG. 7C is an end view of the adapter 700 of FIGS. 7A-7B looking toward the proximal end 712 thereof. FIG. 7D is an end view of the adapter 700 of FIGS. 7A-7C looking toward the distal end 718 thereof. FIG. 7G is a side view of the adapter 700 showing striations 719 which may optionally be applied thereto. Striations 719 may optionally be applied to the outer wall of the adapter 700 to improve the strength of the wall and to provide a stable gripping surface when threading the adapter onto a mating fitting. FIG. 7E is a side stylized transparent view of the adapter 700 of FIGS. 7A-7D as it may be used with an example embodiment 730 of an indicator according to an aspect of the invention, prior to assembly of the adapter 700 on the indicator 730. FIG. 7F is a side stylized transparent view of two instances of the adapter 700 of FIGS. 7A-7D as they may be assembled onto each end of an example embodiment 740 of an indicator according to an aspect of the invention. As best seen in FIGS. 7E and 7F, the adapter 700 may be used, for example, with an indicator 100, or any similar indicator 730 or 740, adapt the Luer lock fitting (such as the lock fitting 134 of FIGS. 1C, 1F, and 1G) on the proximal end to a catheter tip connection. The adapter 700 may also be used, for example, to adapt the catheter tip connection on the distal end 102 to a Luer lock fitting. As best seen in FIG. 7F, two adapters could be used on the same indicator. The adapter could also be used with other devices. The adapter provides a sufficiently good seal to accommodate the pressure or vacuum/suction encountered in a clinical environment.

FIG. 8A is a partial cutaway view of another example embodiment 800, according to a further aspect of the invention, of apparatus for obtaining and containing a fluid obtained from a person or animal, measuring or detecting one or more characteristics of the fluid, and displaying an indication relating to the measurement. FIG. 8B is an exploded view of the indicator 800. Indicator 800 has a generally tubular housing 814 and an end cap 822. The indicator 800 has a distal end 810 on the housing and a proximal end 818 on the end cap 822. End cap 822 serves to close and seal the tubular housing 814 and furnishes a fluid flow connection to the fluid flow passage 816. A fluid flow passage 816 extends between respective intake and outflow ports at the distal end 810 and the proximal end 818. A diffuser 840 is disposed in an enlarged chamber or portion 820 of the fluid flow passage 816. A first indicating element 830 may be formed as a tube or roll and be disposed coaxially with or around the diffuser 840. A second indicating element 832 may also be formed as a tube or roll and be disposed coaxially with the diffuser at a location further from the source of fluid being tested than the first indicating element. The diffuser is built up from a plurality of blocks 842 disposed in series. FIG. 8C is an exploded view showing two instances of blocks 842 from which the diffuser 840 is built up. Each block has one or more apertures 844, such that, in the built-up form, the respective apertures 844 of the plurality of blocks 842 are columnar-aligned. Each columnar-aligned group of apertures forms a fluid-flow conduit, passage, or lumen extending the length of the built-up diffuser 840. Spacers 846 provided on the blocks preclude a seal between adjacent blocks, such that fluid may leak in or out of the conduits or lumina. The diffuser 840 seals against the distal end 848 of the chamber 820.

In operation, the intake port at the distal end 810 of the indicator 800 is connected to a fluid transport source, and source of vacuum or suction is applied to the outflow port at the proximal end of the end cap 822. Under the influence of vacuum or suction in chamber 820, fluid is drawn through the distal end port through passage 816 and then through the conduits or lumina in the center of the diffuser 840. Fluid flows from the central conduits or lumina outward through the spaces between blocks 842 toward and in contact with the first and second indicating elements 830 and 832. The fluid continues along the outer wall of the chamber 820 through the end cap 822 and exits through the proximal end port to the vacuum or suction system.

The first and second indicating elements 830 and 832 may be implemented as different portions of a single object or strip of indicating medium, as depicted in FIG. 8B, or could be implemented as separate objects or indicating media. The first and second indicating elements 830 and 832 may be constructed of indicator media of any of the types and characteristics described earlier in connection with the first and second indicating elements, e.g., elements 108 and 210 of FIGS. 1A, 1D, and 1E, 5016 and 5020 of FIG. 50. This configuration of first and second indicating elements corresponds to the first schematic arrangement 5010 of FIG. 50. The reference symbol “Z” is used in the Figures generally to denote one or more instances of the second indicating element where the second indicating element is a part of a cylindrical or tubular integrated indicating medium object that includes both the first and second indicating element, is disposed about a diffuser contained inside an indicator device, and is located further from the source of fluid being tested than the first indicating element.

FIG. 9 is a cross-section view of another example embodiment 900, according to a further aspect of the invention, of apparatus for obtaining and containing a fluid obtained from a person or animal, measuring or detecting one or more characteristics of the fluid, and displaying an indication relating to the measurement. Indicator 900 may be particularly suitable for use where a source of continuous vacuum or suction is not available, where it is desirable to filter the fluid before reaching the indicating elements, or where any of the indicating elements is of a type which requires that positive pressure be present to force the fluid to saturate or impregnate the indicator element. Indicator 900 has a housing 910 and a diffuser element 918 which may be generally similar to those of the housing 110 and diffuser 120 of FIGS. 1A-1E, with certain exceptions noted hereunder. A distal end 912 provides a port which opens into a fluid passage 914. A proximal end 920 provides a port which opens into a fluid passage or chamber 922. A valve 916 is provided in the fluid passage 914 and permits fluid flow only in the direction from the distal end port to the proximal end port. A plunger 924 is provided in the chamber 922. The plunger 924 may be used to draw or aspirate fluid from a fluid transport source through fluid passage 914 and valve 916 into a chamber 940 in the indicator section 942 of the housing. The plunger 924 may also be used to create positive pressure in the chamber 940 after aspiration of fluid. A filter 932 optionally may be disposed concentrically on the outside of the diffuser in the indicator section. The filter 932 may be used, for a non-limiting example, where the fluid under test is whole blood, and it is desirable that only plasma reach any of the indicating elements, substantially excluding red blood cells therefrom. The plasma has certain proteins that may adhere to antibodies present in one or more of the indicating elements—i.e., such that one or more of the indicating elements is sensitive to certain proteins in the plasma—and cause a color change.

The filter could also exclude particles or other components from reaching one or more of the indicator elements. The fluid may have components that vary in density, viscosity, flow characteristics, and may bear particulates, solids, or gel globules of various sizes, and other differences. The filter may operate as a mechanical filter such as a mesh, screen, or other media, but could also employ other means of selecting or deselecting or differentially passing components of the fluid, including without limitation electrical charge, magnetic fields, chemical bonds, receptors or other means, fluid dynamics, size, shape, density, viscosity, or the like.

As best seen in FIG. 9, the indicating element section 942 comprises a first indicating element section 944 and a second indicating element section 946. A first indicating element 934 may further be disposed concentrically on the outside of the filter 932, if present, or outside of the diffuser is no filter is present, within the first indicating element section 944. A second indicating element 948 may be disposed concentrically on the outside of the filter 932, if present, or outside of the diffuser is no filter is present, within the first indicating element section 944. The second indicating element 948 is located further from the source of fluid being tested than the first indicating element. The first and second indicating elements 934 and 948 may be constructed of indicator media of any of the types and characteristics described earlier in connection with the first and second indicating elements, e.g., elements 108 and 210 of FIGS. 1A, 1D, and 1E, 5016 and 5020 of FIG. 50, and 5116 and 5120 of FIG. 51. The reference symbol “U” is used in the Figures generally to denote one or more instances of the second indicating element where the second indicating element is a part of a cylindrical or tubular indicating medium used with a filter, and is located further from the source of fluid being tested than the first indicating element.

One or more of the indicating elements may be observed through an indicator window 938 and compared to a reference indicator 936 to obtain an indication, reading, or measurement. Pressure created by the plunger 924 forces fluid through the filter 932 and into the indicating elements 934 and 948. FIGS. 12A, 12B, and 12C depict, in schematic form, a further example embodiment of an indicator having a filter and the capability of using positive pressure in the housing to force fluid though the filter 932 and toward the indicating element. Any of the aforementioned indicators could be modified to incorporate a filter as generally shown in FIG. 9.

The shape of the indicator window may act as a magnifying lens to allow a small medium to be easily viewed. The position and size of the indicating element is preferably selected such that the condition of the indicating element is visually apparent when fluids are initially aspirated or drawn into the indicator so that the user need not take any additional steps in order to obtain a measurement. For example, when an indicator is used in an application where pH is sensed to confirm correct insertion of the nasogastric tube in the patient's stomach, the sizes and positions of the indicating element and indicator window are preferably selected so that the pH indication (or a simpler correct insertion indication that the pH is below a threshold which condition is accepted as evidence of correct insertion) is clearly observable to a user upon or shortly after aspiration of the fluid sample in the indicator without requiring the user to undertake additional steps to ascertain the measurement. However, in other applications, the user may be required to perform one or more additional operations or manipulations of or related to the indicator in order to obtain a measurement.

A suitable reference indicator, such as reference indicators 116 of FIGS. 1A, 1D, and 1E, 546 of FIG. 1F, or 936 of FIG. 9 may be provided to guide a user in interpreting the indications provided by the indicating element. In some cases, the number, pattern or complexity of the indications which may be displayed by the indicating element does not permit a comprehensive guide to interpretation to be presented in the space available on the reference indicator provided on the housing or other component of the indicator. Plural reference indicators may be provided on the housing or other component of the indicator. Also, the on-device reference indicator may be replaced by, or supplemented by, a separate reference indicator, which could be, for example, a card, nomogram, pamphlet, or the like.

FIG. 10A is a flow chart of an example embodiment of a method 1000 which may be used in conjunction with embodiments of indicators of the types disclosed herein for obtaining a measurement or detection of one or more characteristics of a fluid acquired from a person or animal. The method 1000 may be used with a “source” of vacuum or suction external to the indicator device. Although the several steps of method 1000 are shown in a particular order, some of the steps may be executed in a different order, and some of the steps may be omitted, including some steps which are not explicitly characterized as optional.

In step 1010, the user couples the indicator's intake port to a fluid transport source. This may include attaching a fitting on the fluid transport source to a mating fitting at the intake port, and where the transport source and the intake port are not directly compatible; it may include the use of an adapter, such as adapter 700 or a similar adapter. In step 1012, the user couples the indicator's outflow port to a source of vacuum or suction. The source may, for example, be a tube or other conduit connected to a vacuum or suction system. The vacuum or suction source could also be a suction bulb or syringe or a tube or conduit coupled thereto. This may include attaching a fitting on the outflow port to a mating fitting on the suction source. Where vacuum or suction source and the outflow port are not directly compatible, this may include the use of an adapter, such as adapter 700 or a similar adapter.

In step 1014 the user enables the source of vacuum or suction so as to cause fluid to be drawn into the indicator. This step is optional, in that the vacuum or suction may continuously operate. Controlling the vacuum or suction may be preferable in some applications to avoid having continuous flow past the indicator. As an alternative, a valve could be provided at or near the intake port of the indicator or in the fluid transport source, allowing the user to control the flow of fluid through the indicator. If the source of vacuum or suction is a bulb or syringe, the user may operate the bulb or syringe to cause the fluid to be drawn into the indicator.

In step 1016, the user observes the presence of fluid in or past the indicator. This step is optional. In step 1018, which is optional, the user disables the flow of vacuum or suction, or otherwise stops the flow of fluid through the indicator.

In step 1020, the user obtains a measurement displayed or exhibited or otherwise detectable from the indicator. For example, if the indicating element is operative to display a color change responsive to the presence or amount of a characteristic of the fluid, the user may observe the color of the indicator element, e.g., through the window, and compare it with the reference indicator to obtain the measurement. The measurement could also be obtained by an instrument that senses the display or other reaction of the indicating element. The term “measurement” may include the determination of a continuous-valued result that represents the characteristic (e.g., 5.5), or the determination of a range within which the result falls (e.g., in the range 5 through 6), the determination that the result exceeds a threshold (or does not) (e.g., below 6), the determination that the result corresponds to a specific indication displayed by the indicating element (e.g., the displayed characters “OK”), or another appropriate determination.

In step 1022, the user uncouples the intake and outflow ports from the fluid transport source and the source of vacuum or suction, respectively. In step 1024, the user may safely dispose of the indicator and accessories.

FIG. 10B is a flow chart of an example embodiment of a method 1050 which may be used in conjunction with embodiments of indicators of the types disclosed herein for obtaining a measurement or detection of one or more characteristics of a fluid acquired from a person or animal. The method 1050 may be used with an indicator device that incorporates a syringe or other accessory element to produce suction or vacuum, and pressure, in the indicator device. Method 1050 may be used, for example, with indicator 900 of FIG. 9 and indicators 1200, 1250 of FIGS. 12A, 12B, and 12C. Although the several steps of method 1050 are shown in a particular order, some of the steps may be executed in a different order, and some of the steps may be omitted, including some steps which are not explicitly characterized as optional.

In step 1060, the user depresses the plunger of the syringe (or similar accessory) to ready it for use in drawing fluid into the indicator. In step 1062, the user couples the indicator's intake port to a fluid transport source. This may include attaching a fitting on the fluid transport source to a mating fitting at the intake port, and where the transport source and the intake port are not directly compatible, may include the use of an adapter, such as adapter 700 or a similar adapter.

In step 1064, the user ensures an intake valve is open to allow fluid to be drawn from the fluid transport source. The intake valve may be provided as part of the indicator near the intake port thereof, or in accessory tubing or conduits. This step is optional. In some applications, the valve may not be required. And some valves may operate automatically to allow fluid to flow in only one direction, to enter the indicator, through the intake port.

In step 1066, the user withdraws the plunger of the syringe to draw fluid from the fluid transport source through the intake port and into the indicator. The user may stop operating the plunger when sufficient fluid is observed to occupy the indicator to saturate the indicating element.

In step 1068, the user ensures that the intake valve is closed. This step is optional, as the valve may not be required or may operate automatically.

In step 1070, the user depresses the plunger so as to force fluid through a filter and into contact with or the vicinity of the indicating element. If the intake valve is present, it prevents fluid from exiting through the inflow port, and in conjunction with the operation of the plunger, causes the pressure in the interior of the indicator to increase, thereby forcing the fluid through the filter. If the intake valve is not present, the movement of the fluid under the influence of the plunger may be sufficient to cause fluid to flow through the filter.

In step 1072, the user obtains a measurement displayed or exhibited or otherwise detectable from the indicator. For example, if the indicating element is operative to display a color change responsive to the presence or amount of a characteristic of the fluid, the user may observe the color of the indicator element, e.g., through the window, and compare it with the reference indicator to obtain the measurement. The measurement could also be obtained by an instrument that senses the display or other reaction of the indicating element.

In step 1074, the user uncouples the intake port from the fluid transport source. In step 1076, the user may safely dispose of the indicator and accessories. It is noted that the methods 1000 of FIG. 10A and the method 1050 of FIG. 10B may include a step of observing the second indicating element for a ‘do-not-use’ indication before or after any one of the steps. For example, an indicator may include a second indicating element that is a water-exposure do-not-use indicating element. If at any time in performance of the methods a ‘do-not-use’ indication is observed, the indicator may be disposed and replaced. This may be beneficial to observe the ‘do-not-use’ indication before drawing fluid from the patient.

FIGS. 11A through 11E are simplified cross-section diagrams of several example embodiments of indicating elements, and configurations thereof, which may be used in conjunction with any of the indicators disclosed in this application. The diagrams of FIGS. 11A through 11E generally correspond to instances of first or second indicating elements.

FIG. 11A depicts an example embodiment 1110 having an indicating element 1122 with a general configuration similar to that previously disclosed in connection with indicator 100 of FIGS. 1A, 1D, and 1E. The indicating element 1122 is constructed as a tube or cylinder and is disposed generally coaxially between diffuser 1120 and the generally tubular wall 1124 of the housing. The housing wall 1124 may be transparent to form a window through which the indicating element may be observed. The indicating element 1122 may, for non-limiting example, be realized as a tube or rolled section of a pH-indicating paper. In this configuration, the measuring component or medium, which may for non-limiting example be a pH-sensitive material that changes color responsive to pH, is impregnated as an integral part of the indicating element 1122. Other measuring components or media could also be used as hereinbefore described. The indicating element 1122 could be entirely composed of the measuring component or medium, instead of being formed as a substrate with the measuring component or medium impregnated therein.

FIG. 11B depicts an example embodiment 1130 having an indicating element 1132 with a general configuration similar to that of indicating element 1122 of FIG. 11A. The indicating element 1132 may constructed as a tubular or cylindrical substrate and is disposed generally coaxially between diffuser 1120 and the generally tubular wall 1124 of the housing. However, the measuring component or medium 1134 is disposed on the outer surface of the indicating element 1132. The measuring component or medium 1134 may, for example, be a gel, ink, or other appropriate indicating substance, and may be deposited, printed, adhered with adhesive, or otherwise applied or attached to the indicating element 1132 substrate.

FIG. 11C depicts an example embodiment 1140 having an indicating element 1142 with a general configuration similar to that of indicating element 1122 of FIG. 11A. The indicating element 1142 may constructed as a tubular or cylindrical substrate and is disposed generally coaxially between diffuser 1120 and the generally tubular wall 1124 of the housing. However, the measuring component or medium 1144 is disposed on the inner surface of the indicating element 1142. The measuring component or medium 1144 may, for example, be a gel, ink, or other appropriate indicating substance, and may be deposited, printed, adhered with adhesive, or otherwise applied or attached to the indicating element 1142 substrate. The indicating element 1142 is preferably transparent or translucent so that the state of the measuring component or medium 1144 may be observed by the user. Because the visible side of the measuring component or medium 1154 is opposite the side which is exposed to the fluid under test, it is preferably either permeable to the fluid or sufficiently thin or transparent that the effect of the fluid on the exposed side can be observed on the visible side.

FIG. 11D depicts an example embodiment 1150 in which the measuring component or medium 1154 is disposed on the inner surface of the generally tubular wall 1156 of the housing, which the tubular wall 1156 is disposed generally coaxially about the diffuser 1120. Because the measuring component or medium 1154 is disposed on the inner surface of the tubular wall 1156, a separate substrate need not be provided to support the indicating element. The measuring component or medium 1154 may, for example, be a gel, ink, or other appropriate indicating substance, and may be deposited, printed, adhered with adhesive, or otherwise applied or attached to the inner surface of tubular wall 1156. Because the visible side of the measuring component or medium 1154 is opposite the side which is exposed to the fluid under test, it is preferably either permeable to the fluid or sufficiently thin or transparent that the effect of the fluid on the exposed side can be observed on the visible side.

FIG. 11E depicts an example embodiment 1160 in which the measuring component or medium 1164 is disposed on the outer surface of the diffuser 1168. Because the measuring component or medium 1164 is disposed on the diffuser 1168, a separate substrate need not be provided to support the indicating element. The measuring component or medium 1164 may, for example, be a gel, ink, or other appropriate indicating substance, and may be deposited, printed, adhered with adhesive, or otherwise applied or attached to the diffuser 1168. The measuring component or medium 1164 is preferably visible through the generally tubular wall 1166 of the housing, which the tubular wall 1166 is disposed generally coaxially about the diffuser 1168.

In FIGS. 11B, 11C, 11D, and 11E, the measuring components or media are shown as occupying or covering only a small portion of the circumference of the substrate. However, the measuring components or media may occupy or cover any portion of the substrate, including the entire circumference.

FIG. 12A depicts a simplified schematic cross-section view of further example embodiments 1200, 1250, constructed according to aspects of the invention, of apparatus for obtaining and containing a fluid obtained from a person or animal, measuring or detecting one or more characteristics of the fluid, and displaying an indication relating to the measurement. Indicators 1200, 1250 are generally similar to indicator 900 of FIG. 9 and may be particularly suitable for use where a source of continuous vacuum or suction is not available, where it is desirable to filter the fluid before reaching the indicating element, or where the indicating element is of a type which requires that positive pressure be present to force the fluid to saturate or impregnate the indicator. Indicators 1200, 1250 are similar to one another, differing in the shapes of certain channel-forming structures 1244, 1254 and the drain channels 1246, 1256 formed thereby. FIG. 12A is a view looking toward a sectioning plane extending through a central longitudinal axis of the indicators 1200, 1250. FIG. 12A depicts a half of the view, the reflection about reflection line 1210 providing the otherwise-identical other half. FIG. 12B is a simplified cross-section view of indicator 1200, and FIG. 12C is a simplified cross-section view of indicator 1250, taken along section line 12-12 of FIG. 12A.

Indicators 1200, 1250 have a generally elongate tubular body 1220 with a distal end 1224. The body 1220 defines an interior space including an interior fluid passage 1226 and an indicator section 1236 thereof. The body 1220 provides an intake port leading into the fluid passage 1226 near the distal end 1224. A valve 1228 is optionally disposed in the interior fluid passage 1226 to control fluid passing through the port and the fluid passage 1226. Valve 1228 may be any suitable manually operated valve, such as a stopcock, ball valve, butterfly valve, clamped flexible tube, or the like. Valve 1228 may also be any suitable automatically operating valve that permits fluid to flow through the interior fluid passage 1226 substantially only one-way, into the indicator. For example, valve 1228 may be a flap valve, a needle valve, a duckbill valve, check valve, or any other appropriate valve. A diffuser 1230 of any of the types disclosed heretofore may be disposed in fluid passage 1226.

Fluid passage 1226 preferably includes a syringe section 1222 disposed near the proximal end of the body 1220. A plunger 1234 having a piston 1232 in the syringe section 1222 may be operated to provide positive pressure or negative pressure (suction or partial vacuum) in the fluid passage 1226 on the distal side of the piston.

A filter 1238 may be provided to define a filtered fluid passageway and chamber 1218 to the radial outside of the filter. Filter 1238 may, for example, be a fluid-permeable cylindrical filter disposed generally coaxially with the diffuser 1230 and the body 1220, and interposed between fluid passage 1226 and chamber 1218, allowing fluid to pass therebetween. The indicators 1200, 1250 have a first indicating element 1240 disposed in the chamber 1218 within a first indicating element section 1670, and a second indicating element 1674 disposed in the chamber 1218 within a second indicating element section 1672. The second indicating element 1674 is located further from the source of fluid being tested than the first indicating element 1240. The filter 1238 may be constructed using any appropriate filter medium as needed to allow those fluid components to which it is desired to expose the indicating element 1240 to pass, while excluding undesired fluid components. The filter 1238 may be used, for non-limiting example, where the fluid under test is whole blood, and it is desirable that only plasma reach one or more of the indicating elements, substantially excluding red blood cells therefrom. The indicating element may be sensitive to certain proteins in the plasma. The filter could also exclude particles or other components from reaching one or more of the indicating elements. The first and second indicating elements 1240 and 1674 may, for example, be implemented as sections of a cylindrical or tubular indicating medium element disposed generally coaxially with the diffuser 1230 and the body 1220, to the radial outside of filter 1238. The first and second indicating elements 1240 and 1674 could also be implemented as separate cylindrical or tubular indicating medium elements, or could take any other form appropriate for the shape of chamber 1218. The first and second indicating elements 1240 and 1674 may be constructed of indicator media of any of the types and characteristics described earlier in connection with the first and second indicating elements, e.g., elements 108 and 210 of FIGS. 1A, 1D, and 1E, 5016 and 5020 of FIG. 50, and 5116 and 5120 of FIG. 51. The configuration of first and second indicating elements described in connection with FIGS. 12A, 12A2, 12B, and 12C corresponds to the first schematic arrangement 5010 of FIG. 50.

One or more fluid-channel forming structures 1244 (FIG. 12B) or 1254 (FIG. 12C) are preferably provided on or the interior of the wall 1242 in the area of indicator section 1236. The fluid-channel forming structures 1244 and 1254, may, for example, be constructed as longitudinal ribs, ridges, striations, or corrugations, forming fluid channels 1246 between the individual ones of the structures. As best seen in FIG. 12B, fluid-channel forming structures 1244 may have rounded edges. As best seen in FIG. 12C, fluid-channel forming structures 1254 may have square or rectangular cross sections or other similar non-rounded profile. The fluid channels conduct fluid which has flowed through or around first indicating element 1240 and is present in the space 1216 to the radial outside of the first indicating element 1240 to a drain passage 1248. As best seen in FIG. 12A, drain passage 1248 is constructed to exhaust into the proximal syringe section of fluid passage 1226. However, drain passage 1248 could instead drain to an external drain, such as a drain tube (not shown).

In operation, the plunger 1234 is depressed in the syringe section 1222 to expel the air to the distal side of the piston 1232. Then the indicator 1200 or 1250 is coupled to the fluid transport source. The plunger 1234 is withdrawn to draw fluid through the distal, fluid intake port, valve 1228, and fluid passage 1226, preferably to fill the indicator section 1236 thereof. The valve 1228, if present, is closed to prevent fluid from escaping from the fluid intake port. Then plunger 1234 is again depressed to pressurize the fluid in fluid passage 1226, including indicator section 1236 thereof. If valve 1228 is an automatically-operating valve, it may close when the fluid passage 1226 is pressurized. Under the influence of this pressure, fluid is forced through the filter 1238. The filtered fluid is forced into the areas 1218, 1216 around indicating elements 1240 and 1674, and then through fluid channels 1246, 1256, drain passage 1248, and then into the syringe section 1222 on the proximal side of piston 1232. Thus, fluid channels 1246, 1256 and drain passage 1248 relieve the pressure created by plunger 1234 in fluid passage 1226 by allowing the fluid to escape. The pressure created by plunger 1234 may be needed to overcome resistance to fluid flow of filter 1238.

FIG. 12A2 depicts a simplified schematic cross-section view of further example embodiments 1680 and 1682, constructed according to aspects of the invention, of apparatus for obtaining and containing a fluid obtained from a person or animal, measuring or detecting one or more characteristics of the fluid, and displaying an indication relating to the measurement. Indicators 1680 and 1682 of FIG. 12A2 are generally similar to indicators 1200 and 1250 of FIG. 12A, respectively, as heretofore described, and except for the differences explained below, the foregoing descriptions of indicators 1200 and 1250 are applicable to indicators 1680 and 1682, mutatis mutandis, are hereby incorporated by reference, and therefore, only principal differences are described. Indicators 1680 and 1682 differ from indicators 1200 and 1250, respectively, in the locations of the second indicating element section 1692 and the second indicating element 1694. Indicators 1680 and 1682 are similar to one another, differing in the shapes of certain channel-forming structures 1244, 1254 and the drain channels 1246, 1256 formed thereby.

As best seen in FIG. 12A2, indicators 1680 and 1682 have a generally elongate tubular body 1220 with a distal end 1224. The body 1220 defines an interior space including an interior fluid passage 1226 and an indicator section 1236 thereof. A diffuser 1230 of any of the types disclosed heretofore may be disposed in fluid passage 1226. A filter 1238 may be provided to define a filtered fluid passageway and chamber 1218 to the radial outside of the filter. Filter 1238 may, for example, be a fluid-permeable cylindrical filter disposed generally coaxially with the diffuser 1230 and the body 1220, and interposed between fluid passage 1226 and chamber 1218, allowing fluid to pass therebetween. A first indicating element 1240 is disposed in the chamber 1218 within a first indicating element section 1682. One or more fluid-channel forming structures 1244 (FIG. 12B) or 1254 (FIG. 12C) are preferably provided on or the interior of the wall 1242 in the area of indicator section 1236. The fluid-channel forming structures 1244 and 1254, may, for example, be constructed as longitudinal ribs, ridges, striations, or corrugations, forming fluid channels 1246 between the individual ones of the structures. The fluid channels conduct fluid which has flowed through or around first indicating element 1240 and is present in the space 1216 to the radial outside of the first indicating element 1240 to a drain passage 1248. As best seen in FIG. 12A2, drain passage 1248 is constructed to exhaust into the proximal syringe section of fluid passage 1226. A second indicating element 1694 is disposed in the drain passage 1248 within a second indicating element section 1684. In other respects, indicators 1680 and 1682 are similar to indicators 1200 and 1250, respectively, including the mode of operation, heretofore described.

FIG. 13A depicts a partly-exploded side view of a further embodiment 1310 of apparatus for obtaining and containing a fluid obtained from a person or animal, measuring or detecting one or more characteristics of the fluid, and displaying an indication relating to the measurement, in accord with a further aspect of the invention. Indicator 1310 is generally similar to the indicator 100 of FIGS. 1A, 1D, and 1E hereinbefore described, with a housing 1312, and a diffuser 1318 which includes a distal, outflow, or exhaust port 1320 for coupling to a source of suction or vacuum. Housing 1312 and diffuser 1318 may be constructed and have functions similar to those of the housing and diffuser of indicator 100. In the assembled form of indicator 1310, diffuser 1318 is telescopically disposed in housing 1312.

The diffuser 1318 preferably has an indicating element 1322 supplied in a quantity larger than that needed for a single use. For example, the indicating element 1322 may be wrapped as a roll of several turns or layers around an outer portion or wall 1324 of the diffuser 1318 that is normally contained within the interior space of the housing 1312. Alternatively, the indicating element 1322 may be formed as a roll of several turns or layers and generally disposed in an interior storage space of the diffuser 1318. The indicating element 1322 may extend through a slit in the wall 1324 of the diffuser and then wrap around a portion of the wall 1324. In operation, fluid in the interior space of the housing 1312 exposes the part of the indicating element 1322 that is on the outside of wall 1324. A gasket or other seal may be provided at the slit to prevent fluid from exposing the portion of the indicating element contained within the interior storage space of the diffuser. A further slit 1314 may be provided in the housing 1312. A used or expended portion of the indicating element 1322 may extend through the slit 1314 to the outside of the housing 1312 where it may be examined or discarded. A gasket or seal may also be provided for slit 1314 to avoid leakage of the fluid out of the indicator, or drawing air into the indicator under the influence of vacuum or suction.

The exposed part of the indicating element 1322 could also be viewed through an indicator window (not shown), as with other embodiments of indicators hereinbefore described. The diffuser 1318 may be rotatable with respect to housing 1312 to cause an exposed portion of the indicating element 1322 to appear in the window.

After a measurement is taken, the used or expended portion of the indicating element 1322 may be drawn through the slit 1314 so as to pull an unexposed portion of the indicating element 1322 from the internal storage compartment of the diffuser 1318, readying the unexposed portion for use. Portions of the indicating element 1322 may be defined by perforations, e.g., to allow one or groups of such portions to be easily separated and removed. Measurements using newly-exposed portions of the indicating element 1322 may be observed in rapid succession by repeatedly drawing fresh, unexposed portions of the indicating element into the fluid-filled space inside within the housing. The indicator 1310 may be drained between uses, allowing measurements to be taken at different times.

FIG. 13B is a side view of an example embodiment 1330 of a cassette constructed according to an aspect of the invention for storing a supply of indicating element sheets. The cassette 1330 could be used in conjunction with the indicator 1310 of FIG. 13A to replace the rolled indicating element 1322. The cassette 1330 could be disposed within the diffuser 1318. Alternatively, a portion of the body of the diffuser could be omitted and the cassette 1330 could occupy that space within the housing 1312. The cassette 1330 may have a body 1332 which houses respective supplies of first indicating element sheets 1334 and second indicating element sheets 1336. Although two groups of such sheets are shown, the cassette could house any number of sheets in any number of groups. In operation, the sheets may be withdrawn through slits, such as 1314 and 1326, as with indicator 1310. Successive sheets may be folded in an interlocked arrangement so that the complete removal of one sheet causes a limited portion of the following sheet to extend through one or more of the slits to serve as a handle.

FIG. 13C depicts a partly-exploded side view of a further embodiment 1350 of apparatus for obtaining and containing a fluid obtained from a person or animal, measuring or detecting one or more characteristics of the fluid, and displaying an indication relating to the measurement, in accord with a further aspect of the invention. Indicator 1350 is generally similar to the indicator 100 of FIGS. 1A, 1D, 1E, and 1F, hereinbefore described, with a housing 1352 and a diffuser (not shown). The housing 1352 preferably has an indicator window 1364. An indicating element tray 1356, preferably containing a stacked supply of indicating element sheets 1358, is arranged in a manner similar to a printer paper tray. The indicating element tray 1356 may be installed in the housing 1352 so as to be visible in the indicator window 1364. The indicating element sheets 1358 may have a fluid impermeable substrate, and the tray and sheets may be cooperatively arranged so that only the top-most sheet can be exposed by fluid in the interior space of the housing 1352. Expended sheets may be removed, and a new sheet readied for use, by manually removing the diffuser from the housing, and removing the tray 1356 for access. Then the tray and diffuser may be replaced. Alternatively, a slit 1314 may be provided through which the used sheet may extend and be removed.

FIGS. 13D and 13E depict a further example embodiment 1370 of an indicator which may be constructed according to another aspect of the invention. Indicator 1370 has a housing 1372 which may be constructed in a manner similar to housings of indicator embodiments as hereinbefore described. However, indicator 1370 differs in that housing 1372 preferably has a first indicating element 1374 and a second indicating element 1376, and an indicator window 1384 selectively rendering visible a limited subset of the indicating elements, such as indicating element 1376. Housing 1372 preferably also has one or more fluid channels (not shown) adapted to expose to the fluid drawn into the housing only that limited subset of indicating elements visible in the indicator window 1384. Different ones of the indicating elements may thus be selectably exposed to the fluid, observed, and a measurement taken therefrom. FIG. 13E depicts a diffuser 1380 which may be used in conjunction with the housing of FIG. 13D. The diffuser 1380 has a distal or exhaust port 1382 for coupling to a source of suction or vacuum. A body portion 1378 of the diffuser 1380 is adapted for telescopic arrangement in housing 1372. First and second indicating elements 1374 and 1376 are disposed at various angular positions on the body portion 1378. Diffuser 1380 and housing 1372 may be adapted for rotation with respect to one another so as to selectively render visible the indicating elements 1374 and 1376 in the indicator window 1384.

Although the foregoing descriptions of FIGS. 13A-13E refer to diffusers in the form of integrated components having structures to accommodate several functions, including routing the fluid within the housing toward the indicating elements, closing the housing, and containing cassettes, trays, rolls or other supplies of indicating elements, these functions may be provided by separate components.

In some cases, the indicating medium of an indicating element is fragile in some way. For example, some pH papers operate using fluid-characteristic-sensitive dies which can be removed by the action of the fluid passing over or through the paper. The paper itself, once saturated by fluid, can also disintegrate. According to an aspect of the invention, the indicating element may include coatings, membranes, or other structures or treatments to avoid disintegration of the element or its substrate, and avoid separation of the dies or other operating components from the element or its substrate. The chamber or diffuser may also have structures of features to limit fluid velocity or turbulence to minimize disturbance of the indicating element or its substrate.

FIG. 14A is a side cross-section view of a further embodiment 1410 of an indicating element constructed according to an aspect of the invention. The indicating element 1410 includes an indicating medium 1414, which may for non-limiting example be a pH sensitive paper, but could also be any of the other indicating media or measurement media discussed elsewhere herein. A semi-permeable membrane 1412 surrounds the indicating medium 1414. Although the membrane 1412 is shown on both sides of the indicating medium 1414, it could also be provided on only one side, which may be the side facing or exposed to the fluid, or a side that does not face the fluid, if the mechanical arrangement of the indicating element so permits. Fluid 1416 flows through the membrane 1412 to expose or saturate the indicating medium 1414. The membrane may be positioned adjacent to the pH indicator or may form a coating. The membrane helps prevent washing, diluting or dissolving of pH indicator color dye. The membrane can include, but is not limited to, gel, sponge, foam, plastic, and may be formed mechanically (e.g., as a piece of material with micro holes drilled through it). The membrane could also be a stretched PTFE material of the type used in rain clothing. The membrane would also allow gastric fluid to permeate through the barrier to achieve saturation, but provide a mechanical barrier for to prevent dyes from washing out.

FIG. 14B is a simplified cross-section view of a further example embodiment of an indicator 1420 constructed according to the invention, which may be similar to the embodiments of FIGS. 1A, 1D, and 1E but for modifications explained here. Indicator 1420 has in an enclosure 1432, which may be formed as part of the housing or may be a separate piece. The enclosure 1432 forms a space or chamber 1428. A first indicating element 1424 resides within a first indicating element section 1450 of space or chamber 1428. A second indicating element 1454 resides within a second indicating element section 1452 of space or chamber 1428. The second indicating element is located further from the source of fluid being tested than the first indicating element. In operation, fluid 1422, under pressure supplied from a source, or under the influence of vacuum applied at port 1456, flows through the space or chamber 1428 in which the indicating elements reside. An air exit port 1426 allows air to leave the space, but impedes to at least some extent the flow of fluid, which may also take other paths. Thus the fluid is impeded or retarded in flowing past the indicating elements, thereby avoiding disturbing its structure or removing dye therefrom. The first and second indicating elements 1424 and 1454 may be constructed of indicator media of any of the types and characteristics described earlier in connection with the first and second indicating elements, e.g., elements 108 and 210 of FIGS. 1A, 1D, and 1E, 5016 and 5020 of FIG. 50, and 5116 and 5120 of FIG. 51. The reference symbol “S” is used in the Figures generally to denote one or more instances of the second indicating element where the second indicating element is located in a chamber having a fluid-flow-restricting exit port, and is located further from the source of fluid being tested than the first indicating element.

FIG. 14C is a simplified cross section view of a further example embodiment 1440 of an indicator constructed according to the invention, which may be similar to the embodiments of FIGS. 1A, 1D, and 1E but for modifications explained here. A diffuser of one of the types earlier described is shown at 1444. A mechanical barrier 1446 is provided in the fluid path, and is located closer to the proximal end of the indicator device. The mechanical barrier 1446 may be an inline spiral barrier. The indicator element (not shown) may surround the diffuser. Fluid 1442 flows past the diffuser to expose or saturate the indicator element, but is then stopped or impeded by the mechanical barrier 1446, which thereby helps avoid disturbing the structure of or removing die from the indicating element.

FIG. 52 is a side elevation view of a combination of apparatus 8300 comprising any of the fluid characteristic indicators disclosed herein (collectively denoted by reference number 8310), a tee adapter 8200, and other equipment, for use in obtaining measurements of characteristics of fluid, such as that which may be obtained from a human or veterinary patient, or any other source. A first end tubing connector 8230 of tee adapter 8200 is connected to a source 8330 of fluid to be measured, such as a nasogastric tube. A bottom end connector 8314 of fluid characteristic indicator 8310 is inserted into accessory port 8222 of tee adapter 8200. A syringe 8320 is coupled to indicator 8310 by inserting its fluid port 8322 into the top end port 8312 of the indicator 8310. Plunger 8324 may be raised to aspirate fluid from fluid source tube 8330 into indicator 8310.

Indicator 8310 has a first indicating element 8344 and a second indicating element 8346. The second indicating element 8346 is located further from the source of fluid being tested than the first indicating element. When aspirated, the fluid saturates the first and second indicating elements 8344 and 8346. The indicating elements 8344 and 8346 may be compared to reference samples of reference indicator 8340 by viewing them through reference indicator window 8342. The tee adapter 8200 may be reused for multiple measurements by connecting a new fluid characteristic measurement apparatus, e.g., using the accessory port 8222, for each measurement. The user of the tee adapter 8200, and its reuse for multiple measurements, may help retain bodily fluids within the adapter, fluid characteristic measurement apparatus and related accessories and tubing, thereby helping to avoid exposure of personnel to bodily fluids. The reference symbol “T” is used in the Figures generally to denote one or more instances of first and second indicating elements where the indicating elements are visible in the same indicator window with an adjacent reference indicator.

FIG. 52 depicts several other locations where the second indicating element may be placed. The second indicating element may be placed, alternately or additionally, at the originally described location denoted by reference number 8346, or any one or more of these locations.

As denoted by reference number 8350, the second indicating element may be placed at a location on the indicator 8310 displaced from the indicator window 8342. The body of the indicator is preferably transparent, or a transparent window is preferably provided, to allow the second indicating element to be viewed therethrough. The reference symbol “0” is used in the Figures generally to denote one or more instances of the second indicating element where the second indicating element is housed in an indicator that also houses the first indicator, and the second indicator is displaced from an indicator window in which the first indicating element is visible.

As denoted by reference number 8352, the second indicating element may be placed on the neck of syringe 8320. For example, the second indicating element may be realized as an indicating-medium coating on the inside surface of the syringe neck. The syringe neck is preferably transparent, or a transparent window is preferably provided, to allow the second indicating element to be viewed therethrough. The reference symbol “Q” is used in the Figures generally to denote one or more instances of a second indicating element used as part of an assembly comprising an indicator and a component that mates with the indicator, where the second indicating element is located on the mating part of the component.

As denoted by reference number 8354, second indicating element may be placed in the body of syringe 8320. For example, second indicating element may be realized as an indicating-medium coating on the inside surface of the syringe body. The syringe body is preferably transparent, or a transparent window is preferably provided, to allow the second indicating element to be viewed therethrough. The reference symbol “P” is used in the Figures generally to denote one or more instances of a second indicating element used as part of an assembly comprising an indicator and a component that mates with the indicator, where the second indicating element is located on a part of the component other than the part that mates with the indicator.

The first and second indicating elements 8344, 8346, 8350, 8352, and 8354 may be constructed of indicator media of any of the types and characteristics described earlier in connection with the first and second indicating elements, e.g., elements 108 and 210 of FIGS. 1A, 1D, and 1E, 5016 and 5020 of FIG. 50, and 5116 and 5120 of FIG. 51.

FIG. 53 is a set of three schematic depictions of an indicator constructed according to an aspect of the invention wherein the indicator includes a movable cover to aid a user in interpreting information displayed by a second indicating element thereof so as to obtain a measurement or indication from a first indicating element when that measurement or indication is ready. The indicator is shown in an arrangement, assembly, or system of components, including certain environmental components with which the indicator may be used in operation. In the first depiction, the arrangement 1710 is shown with the movable cover omitted. In the second depiction, the arrangement 1740, which is otherwise equivalent to arrangement 1710, is shown with the movable cover is in a first position. In the third depiction, the arrangement 1750, which is otherwise equivalent to arrangements 1710 and 1740, is shown with the movable cover is in a first position.

Each of arrangements 1710, 1740, and 1750 include a source 1712 of fluid to be tested, an indicator 1716, and a source of suction or vacuum 1726. The fluid source 1712 is in fluid communication with indicator 1716 via a fluid conduit 1714, and the indicator 1716 is in fluid communication with suction or vacuum source 1726 via a fluid conduit 1724. Indicator 1716 is generally similar to the indicator 100 of FIGS. 1A-1E as heretofore described, and the foregoing description of indicator 100 is applicable to indicator 1716, mutatis mutandis, and is hereby incorporated by reference. Indicator 1716 has a first indicating element 1720 and a second indicating element 1722 in distinguishable, externally-visible locations. A cover 1732 (omitted from the depiction of arrangement 1710) is adapted for movement from a first position 1728, in which it shields from external view the first indicating element 1720, to the second position 1730, in which it shields from external view the second indicating element 1722. Thus, the cover 1732 is adapted to alternately shield from external view either the first indicating element 1720 or the second indicating element 1722.

A one-way stop 1718 allows the cover 1732 to move from the first position to the second position, but once the cover has been moved to the second position, the stop prevents the cover from moving back to the first position. In the schematic depictions of FIG. 53, the movement is along the longitudinal axis of the indicator. The stop 1718 is shown as a wedge with a ramp surface that the cover may ride over when moving from the first position to the second position, and a vertical blocking surface that interferes with the cover when the cover urged to move from the second position to the first position. Either or both of the cover 1732 and the stop 1718 may be adapted for movement in a direction normal to the longitudinal axis, and either or both of the cover and the top may be equipped with components (not shown), such as a spring, to urge the cover and the stop to bear against one another to aid the effective operation of the stop. The prevention action of the stop 1718 need not be absolute; the stop may simply make it difficult to urge the cover from the second position to the first position, and the stop may be adapted to allow a user intentionally to defeat the prevention action. The stop could be realized using other appropriate structures, including a ratchet or latch device.

In the arrangement 1740, the cover 1732 is shown in the first position 1728. In an example embodiment, the second indicating element 1722 is selected and so located within the indicator 1716 that when fluid reaches the second indicator, the fluid necessarily has also reached and exposed or saturated the first indicator in a way that an indication then produced by the first indicator is ready for use and may be relied upon. In operation of the example embodiment, the cover is preferably initially located in the first position, whereby the first indicating element 1720 is not externally visible, but the second indicating element 1722 is externally visible. The suction source 1726 is operated to draw fluid from source 1712 into the indicator 1716. Upon observing that second indicating element 1722 has been activated to show its indication that fluid has reached it, and therefore that an indication from the first indicating element 1720 may be relied upon, the user moves the cover 1732 from the first position 1728 to the second position 1730, e.g., as shown in arrangement 1750. This has the effect of revealing the first indicating element 1720, so that the user may make an observation or measurement from the first indicating element. In the second position, the cover also shields from view the second indicating element 1720, preventing the user from confusing an indication from the second indicating element as though that indication was from the first indicating element.

Although the schematic depictions of the indicator and cover shown in FIG. 53 show the first and second indicating elements displaced longitudinally, and therefore the cover operates by moving longitudinally, other configurations of the indicating elements and the cover could also be used. For example, the first and second indicating elements could be arranged so as to be visible at different angles about the longitudinal axis of the indicator, and the cover could be adapted to rotate about the axis and be equipped with a window that alternately presents the first or second indicating element for view.

The first and second indicating elements 1720 and 1722 may be constructed of indicator media of any of the types and characteristics described earlier in connection with the first and second indicating elements, e.g., elements 108 and 210 of FIGS. 1A, 1D, and 1E, 5016 and 5020 of FIG. 50, and 5116 and 5120 of FIG. 51.

FIGS. 54-60 are detail views of additional example embodiments of diffusers showing example arrangements of a second indicating element and certain structures or features related to its function. The diffusers shown in FIGS. 54-60 and discussed in connection therewith are generally similar to diffuser 120 of FIGS. 1A-1E, and diffuser 310 of FIGS. 3F and 3H, as heretofore described, and except for the differences explained below, the foregoing descriptions of diffusers 120 and 310 are applicable to diffuser 360, mutatis mutandis, and are hereby incorporated by reference. Accordingly, in the discussion of the diffusers of FIGS. 54-60, only the particular components needed to understand the second indicating element or related structures and features will be specifically discussed.

FIG. 54 is a detail view of a further example embodiment of a diffuser 1760. Diffuser 1760 has a collar 1774, connection tube 1762 extending from one side of the collar, and a generally cylindrical proximal extension 1776 of the connection tube extending from the opposite side of the collar. A fluid communication chamber, lumen, or passage 1778 extends through the interior of the extension 1776, collar 1774, and connection tube 1762. Locator structures 1766 adapted to mate with corresponding structures on a nesting housing component (not shown) of an indicator device are provided on the outer surface 1764 of the extension 1776. An indicator element of the type generally referred to herein as a second indicator element 1768 is provided on the outer surface of the extension 1776 in a second indicating element section or region 1770 near the proximal end 1772 of the extension. The second indicator element 1768 may be located between the proximal ends of the locator structures 1766 and the proximal end 1772 of the extension. The diameter of the wall of the extension forming the outer surface 1764 may be reduced in the area of the second indicating element section 1770 to provide a space for the second indicating element 1768 to reside between the outer surface 1764 and the inner surface of the housing when the housing is mated with the diffuser.

FIG. 55 is a further detail view of the diffuser 1760 of FIG. 54, omitting the second indicating element 1768, so that several alternate or optional structures or features that otherwise would be covered by the second indicating element 1768 may be seen. Any of the following features, which preferably promote fluid flow through or in contact with the second indicating element, may be provided alone or in combination with others of the features, and the need for these features may depend on the particular indicating media and substrates selected to implement the second indicating element. Although each feature is shown for example in some small plurality and occupying only a small fraction of the total angular extent of the outer surface 1764 of extension 1776, it will be appreciated that the few examples shown may be replicated in greater number and over a greater or the whole angular extent of the surface. A first set of apertures 1780 near the proximal end of second indicating element section 1770 may optionally be provided in the outer surface 1764 of extension 1776, extending into the fluid communication chamber 1778. A second set of apertures 1786, outside of and proximal to the second indicating element section 1770 may optionally be provided, extending into the fluid communication chamber 1778. A set of notches, grooves, or depressions 1784 may optionally be provided on the outer surface 1764 of the extension along some or all of the longitudinal extent of the second indicating element section 1770. A third set of apertures 1782 may optionally be provided, coinciding with the proximal ends of grooves 1784, and extending into the fluid communication chamber. Any of the apertures, if provided, allow fluid to flow from a fluid chamber of the indicator in the area on the distal end of the extension 1776 into the internal fluid flow chamber 1778. If the second indicating element 1768 is constructed of a porous or pervious material, the fluid may flow through the indicating element so as to enhance the extent or rate of saturation of the indicating element by the fluid. If the second indicating element 1768 is constructed of a non-porous and non-pervious material, the fluid may flow along and in contact with the indicating element so as to enhance the extent or area of contact between the indicating element and the fluid.

FIG. 56 is a detail view of a further example embodiment of a diffuser 1800, which is generally similar to the diffuser 1760 of FIGS. 53 and 54, except for the location and extent of the second indicating element. Except for the differences explained below, the foregoing description of diffuser 1760 is applicable to diffuser 1800, mutatis mutandis, and is hereby incorporated by reference. Accordingly, only the differences particular to the diffuser 1800, the second indicating element, and certain structures or features related to its function, will be described.

Collar 1774 has a distal facing surface 1806. An indicator element of the type generally referred to herein as a second indicator element 1804 is provided on the outer surface of the extension 1776 in a second indicating element section or region 1802 of the extension. The second indicator element 1804 may be located between the distal ends of the locator structures 1766 and the distal surface 1806 of collar 1774. The diameter of the wall of the extension forming the outer surface 1764 may be reduced in the area of the second indicating element section 1802 to provide a space for the second indicating element 1768 to reside between the outer surface 1764 and the inner surface of the housing when the housing is mated with the diffuser. Although the second indicator element 1804 is depicted as extending between the distal surface 1806 of the collar to the distal ends of the locator structures 1766, the second indicator element could extend further, e.g., to the proximal end 1772 of the extension, or less far, e.g., along only part of the locator tabs. The second indicator element also may extend a shorter distance in the proximal direction.

FIGS. 57 and 58 are further detail views of the diffuser 1800 of FIG. 56, omitting the second indicating element 1804, so that several alternate or optional structures or features that otherwise would be covered by the second indicating element 1768 may be seen. Any of the following features, which preferably promote fluid flow through or in contact with the second indicating element, may be provided alone or in combination with others of the features, and the need for these features may depend on the particular indicating media and substrates selected to implement the second indicating element. Although each feature is shown for example in some small plurality and occupying only a small fraction of the total angular extent of the outer surface 1764 of extension 1776, it will be appreciated that the few examples shown may be replicated in greater number and over a greater or the whole angular extent of the surface.

A first set of apertures 1808 (see FIG. 57) or 1814 (see FIG. 58) may optionally be provided in the outer surface 1764 of extension 1776, near the distal ends of the locator structures 1766, and extending into the fluid communication chamber 1778. A second set of apertures 1810 (see FIG. 57) or 1818 (see FIG. 58) may optionally be provided in the outer surface 1764 of extension 1776, at an intermediate distal location between the distal ends of the locator structures and the distal surface 1806, and extending into the fluid communication chamber 1778. A third set of apertures 1820 (see FIG. 58) may optionally be provided in the outer surface 1764, near the distal surface 1806, and extending into the fluid communication chamber 1778. A fourth set of apertures 1812 (see FIG. 57) or 1822 (see FIG. 58) may optionally be provided in the distal surface 1806 of collar 1774, near the outer surface 1764 of extension 1776, and extending into the fluid communication chamber. A set of notches, grooves, or depressions 1816 may optionally be provided on the outer surface 1764 of the extension along some or all of the longitudinal extent of the second indicating element section 1802. Any of the apertures may coincide with the grooves 1816. Any of the apertures, if provided, allow fluid to flow from a fluid chamber of the indicator in the area on the distal end of the extension 1776 into the internal fluid flow chamber 1778. If the second indicating element 1804 is constructed of a porous or pervious material, the fluid may flow through the indicating element so as to enhance the extent or rate of saturation of the indicating element by the fluid. If the second indicating element 1804 is constructed of a non-porous and non-pervious material, the fluid may flow along and in contact with the indicating element so as to enhance the extent or area of contact between the indicating element and the fluid.

FIG. 59 is a detail view of a further example embodiment of a diffuser 1840, which is generally similar to the diffuser 1760 of FIGS. 53 and 54, except for the shapes of certain locator structures adapted to mate with corresponding structures on a nesting housing component of an indicator device and the extent of the second indicating element. Except for the differences explained below, the foregoing description of diffuser 1760 is applicable to diffuser 1840, mutatis mutandis, and is hereby incorporated by reference. Accordingly, only the differences particular to the diffuser 1840, the second indicating element, and certain structures or features related to its function, will be described.

Diffuser 1840 has locator structures 1846 adapted to mate with corresponding structures on a nesting housing component (not shown) of an indicator device on the outer surface 1764 of the extension 1776. An indicator element of the type generally referred to herein as a second indicator element 1844 is provided on the outer surface of the extension 1776 in a second indicating element section or region 1842 near the proximal end 1772 of the extension. The second indicator element 1842 may be located between the proximal ends of the locator structures 1846 and the proximal end 1772 of the extension. The diameter of the wall of the extension forming the outer surface 1764 may be reduced in the area of the second indicating element section 1842 to provide a space for the second indicating element 1844 to reside between the outer surface 1764 and the inner surface of the housing when the housing is mated with the diffuser.

FIG. 60 is a further detail view of the diffuser 1840 of FIG. 59, omitting the second indicating element 1844, so that several alternate or optional structures or features that otherwise would be covered by the second indicating element 1844 may be seen. Any of the following features, which preferably promote fluid flow through or in contact with the second indicating element, may be provided alone or in combination with others of the features, and the need for these features may depend on the particular indicating media and substrates selected to implement the second indicating element. Although each feature is shown for example in some small plurality and occupying only a small fraction of the total angular extent of the outer surface 1764 of extension 1776, it will be appreciated that the few examples shown may be replicated in greater number and over a greater or the whole angular extent of the surface. A first set of apertures 1848 near the proximal end of second indicating element section 1842 may optionally be provided in the outer surface 1764 of extension 1776, extending into the fluid communication chamber 1778. A set of notches, grooves, or depressions 1850 may optionally be provided on the outer surface 1764 of the extension along some or all of the longitudinal extent of the second indicating element section 1842. Any of the apertures may coincide with the grooves 1850. The apertures, if provided, allow fluid to flow from a fluid chamber of the indicator in the area on the distal end of the extension 1776 into the internal fluid flow chamber 1778. If the second indicating element 1844 is constructed of a porous or pervious material, the fluid may flow through the indicating element so as to enhance the extent or rate of saturation of the indicating element by the fluid. If the second indicating element 1844 is constructed of a non-porous and non-pervious material, the fluid may flow along and in contact with the indicating element so as to enhance the extent or area of contact between the indicating element and the fluid.

FIG. 61 depicts a side-view of an indicator with a micro-needle collector obtaining a fluid sample from a fluid source, in accordance with an embodiment of the present disclosure. In particular, FIG. 61 depicts the indicator 100, which may be realized by any of the indicators disclosed herein, disassembled from the micro-needle collector 6100. In some embodiments, the indicator 100 includes an image of an identifier 6110 located in proximity to the indicator window 118. The identifier 6110 may be realized by a barcode, a QR code, a printed serial number or the like. The identifier 6110 may be a unique identifier and associated with only one indicator 100. The identifier 6110 may be linked with a patient's medical record, for example, when the test is shipped or otherwise presented to the patient for use. The identifier 6110 is located in proximity to the indicator window 118 such that both the indicator window 108 and the identifier 6110 are simultaneously identifiable in a single view. Thus, a single image may be captured, for example by a personal electronic device like a smart phone, that includes both the indicator window 108 and the identifier 6110. This enables a remote interpretation of the test results with a single image.

A proximal end 6106 of the micro-needle collector 6100 attaches to the distal end 102 of the housing 110. While depicted with a locking fitting, any number of methods may be utilized to attach the micro-needle collector 6100 to the distal end 102 of the housing 110, such as a Luer lock fitting, a friction seal, threaded connections, adhesives, and the like. The micro-needle collector 6100 includes a plurality of pipettes 6102. When the distal end 6108 of the micro-needle collector 6100 is placed at the source 6104, the pipettes 6102 draw fluid 1416 via a capillary action from a source 6104 to the distal end 102 of the housing 110. The fluid 1416 is then able to proceed through the fluid chamber 1218 to interact with the indicating element sections 108, 210.

The source 6104 may be a person's or patient's skin, such as an inner forearm, a thigh, or the like. The pipettes 6102 make microscopic punctures in the skin and are able to obtain a blood plasma from the patient. Thus the fluid 1416, which in some embodiments is the patient's plasma obtained from the source 6104, proceeds from the micro-needle collector 6100, to the fluid chamber 1218, and to the indicating element sections 108, 210 to detect the characteristics and presence of various chemicals, proteins, particulates, granules, powders, other solids, and the like.

FIG. 62 depicts a partially exploded view of the micro-needle collector, in accordance with an embodiment of the present disclosure. In particular, FIG. 62 depicts the micro-needle collector 6100 having the plurality of pipettes 6102 located inside of the shell 6202. In some embodiments, the micro-needle collector 6100 has a divergent shape. Each of the pipettes 6102 may be tightly packed together and touching each other at the proximal end 6106 of the micro-needle collector 6100. The pipettes 6102 expand apart from each other towards the distal end 6108 of the micro-needle collector 6100. The pipettes 6102 may extend through holes 6210 located in the plate 6204. Thus, the cross-sectional area of the shell 6202 at its proximal end 6106 may be less than the cross-sectional area of the shell 6202 at its distal end 6108. The spacing of the pipettes 6102 at the distal end 6108 allows for sufficient spacing between respective pipettes 6102 when obtaining the fluid. The cross-sectional area of the pipettes 6102 at the proximal end 6106 may be such that it matches a cross-sectional area of the distal end 102 of the housing 110. The distal ends 6206 of the pipettes 6102 extend beyond the plate 6204 to interact with the source 6104. Although not depicted, an end cap may cover the distal end 6108 of the micro-needle collector 6100. This end cap may ensure sterilization of the indicator 100 before use.

In some embodiments, the distal ends 6206 of the pipettes 6102 may retract in to the shell 6202 behind the plate 6204 after the indicator 100 has been used to draw the fluid from the source 6104. Such a retraction prevents the distal ends 6206 of the pipettes 6102 from further interacting with another source 6104 and may assist in making the micro-needle collector 6100 suitable for disposal. The retraction may occur after a user initiates a retraction procedure, for example by twisting the shell or engaging a device to either extend the plate 6204 distally or to retract the pipettes 6102 proximally. In other embodiments, the plate 6204 may extend distally (relative to the pipettes 6102) when disengaging from the source 6104. This may occur due to an adhesive being placed on the distal surface of the plate 6204 that maintains the plate adhered to the source 6104. As the user pulls the indicator 100 away from the source 6104, the pipettes 6102 disengage from the source 6104 while the plate initially stays engaged with the source 6104. This relative motion between the pipettes 6102 and the plate 6204 causes the distal ends 6206 of the pipettes 6102 to retract into the shell 6202 behind the plate 6204. Further pulling of the indicator 100 from the source 6104 causes the adhesive to break away from the source 6104 and the plate 6204 to disengage from the source 6104 with the distal ends 6206 of the pipettes retracted into the shell 6202.

FIG. 63 depicts an end view of the micro-needle collector, in accordance with an embodiment of the present disclosure. In particular, FIG. 63 depicts the distal end 6108 of the micro-needle collector 6100. Extending along the outer ring is the shell 6202. The plate 6204 includes the holes 6210 that respective pipettes 6102 extend through. Each pipette 6102 includes the channel 6302 through which the fluid is carried through the pipette 6102. In some embodiments, the plate 6204 is planar, although in other embodiments, the plate 6204 may have a curvature to conform a contoured shape of the source 6104. Respective lengths of the pipettes 6102 may be altered such that each pipette 6102 extends beyond the plate 6204. A micro-needle collector 6100 with a curved plate 6204 may be coupled with an indicator 100 that requires a larger volume of fluid. The micro-needle collector 6100 with the curved plate 6204 may have more pipettes 6102 than compared to a micro-needle collector 6100 with a planar plate 6204. The additional pipettes may result in an increased rate of obtaining the fluid to reduce a time required to obtain the larger volume of fluid. The curved plate 6204 may be curved to wrap around the curvature of a patient's forearm.

FIG. 64 depicts a block diagram of a system, in accordance with an embodiment of the present disclosure. In particular, FIG. 64 depicts the system 6400 that includes the communication interface 6402, the processor 6404, the memory 6406, and the system bus 6422 to communicatively couple the components of the system 6400. The communication interface 6402 can be any hardware or software that can communicate with a network to communicate with other computer systems, user devices, and the like. The communication interface 6401 can use a variety of protocols, including but not limited to, Session Initiation Protocol (SIP), Ethernet, TCP/IP, UDP, H.323 WebRTC, Short Message Service (SMS), Multimedia Messaging Service (MMS), video protocols, text protocols, Integrated Services Digital Network (ISDN), wireless communication protocols like Wi-Fi and Bluetooth, wired communication protocols, a combination of these protocols, and the like.

The processor 6404 may be any hardware or software configured to process signals and to execute program instructions. The processor 6404 may be realized as a single processor or a plurality of processors and may further include processors located at a variety of locations. The memory 6406 stores information for use by the processor 6404. The memory 6406 may be random access memory (RAM) and/or be a non-volatile hard drive/disk magnetic and/or optical disk memory storage. Generally, the memory 6406 may provide non-volatile storage of computer readable instructions, data structures, program modules, objects, service configuration data and other data for use by the processor 6404.

The memory 6406 includes information for patient demographics 6408, indicator tests 6410, identifier information 6412, and indicator mapping information 6414. The patient demographics 6408 may include any information related to the patient, such as the patient's name, address, health information, and the like. The indicator tests 6410 stores information related to the various tests that may be performed by the indicators 100. For example, the indicator tests 6410 may store information related to how to interpret results from one or both of the first and second indicating elements that may be included with the indicator 100. The identifier information 6412 may store information related to the identifier 6110 on each of the indicators 100. The indicator mapping information 6414 may include a mapping between locations of the first and second indicating elements 108, 210 and their respective locations on the indicator.

The system 6400 may be able to receive an indicator image 6416, an indicator request 6418, and output test results 6420. The indicator image 6416 is an image of the indicator 100 after a test has been performed, such as the image 6700 depicted in FIG. 67. The indicator image 6416 may be captured via a user device (e.g., a smart phone, a digital camera, a web-cam) and transmitted to the communication interface 6402 by way of a MMS message or as an attachment to an e-mail via a cellular or other network. In other embodiments, a user device may have an application installed, the application configured to capture the indicator image 6416 and transmit it to the system 6400. In yet other embodiments, the application may capture the indicator image 6416 and receive information from the memory 6406 to interpret the indicator image 6416 locally on the user device. Thus, the results of interpreting the indicator image 6416 may only be stored locally on the user device without the results being shared or transmitted to another computing device other than the device used by the patient.

In some embodiments, the indicator image 6416 may be realized by a video rather than a still image. A patient may utilize a user device to video capture the performance of the fluid collection with the indicator 100 as well as the indicator image after the fluid collection is complete. Additional data may be obtained to ensure accurate results from the indicator. For example, the video may include voice verification information from the patient, video images depicting proper skin surface preparation, proper placement of the indicator 100 relative to the patient, appropriate time delays are observed, and the like. The indicator image 6416 may also be a video conference between a medical provider and the patient. In the video conference, the medical provider may be able to converse with the patient during the fluid collection with the indicator.

The system 6400 may also be able to output the test results 6420. Outputting the test results 6420 may comprise sending, to the user device, a plain-language interpretation of the test results. Thus, the patient is able to receive near real-time results. Outputting the test results 6420 may further include updating a patient's health record with information from the test results. The information may include the interpretation of the first indicating element section 108, whether the test was a valid test per the second indicating element section 210, a date and time of the results, and the like. The patient's health record may be accessible by one or both of the patient and the patient's medical care provider.

### INDUSTRIAL APPLICABILITY

The systems and methods disclosed herein may be used in a wide variety of applications, particularly in the field of providing diagnostic tests. In one non-limiting example, a patient may be otherwise remote from a medical provider and receiving tele-health services. To provide adequate medical care, the medical care provider providing the tele-health services to the patient may desire for a diagnostic test to be performed on the patient. The diagnostic test may require a fluid sample to be analyzed. However, the patient may be remotely located from a diagnostic testing center, unable to leave their home setting, or the like. The health care provider may cause an indicator 100 to be received by the patient. The indicator 100 is configured with the appropriate indicating elements to perform the diagnostic test. The patient may self-administer the collection of the fluid into the indicator 100 for performance of the diagnostic test. With the use of a user device, such as a cellular phone, the patient is able to capture an image of the indicator and subsequently receive the results of the diagnostic test.

By way of an example, the method 6500 of FIG. 65 is implemented with the indicator 100 and micro-needle collector 6100 of FIG. 61. However, any one of the indicators disclosed herein may be utilized to obtain the fluid to perform the diagnostic test, with the method 6500 being modified for use with the selected indicator.

FIG. 65 depicts a method, in accordance with an embodiment of the present disclosure. In particular, FIG. 65 depicts the method 6500 that includes a plurality of steps, including preparing the skin for a sample at block 6502, attaching a micro-needle collector to the indicator at block 6504, applying the micro-needle collector to the skin at block 6506, capturing an image of the indicator at block 6508, transmitting the image at block 6510, interpreting the image to generate results at block 6512, and outputting the results at block 6514. Although the blocks of the method 6500 are depicted in order, it is understood that the actions of the blocks may be performed out of the depicted order, not all blocks need to be performed, and additional blocks may be added to the method 6500.

FIG. 66 depicts a perspective view 6600 of an indicator 100 attached to a micro-needle collector 6100 obtaining a fluid from a patient forearm 6602. Further, FIG. 67 depicts the image 6700 of the indicator 100 after a fluid has been collected by the indicator. FIGS. 66 and 67 will be utilized in the discussion of the method 6500.

Returning to the method 6500, at block 6502, the skin is prepared for a sample. The skin may be disinfected or sanitized. If necessary or desired, a topical anesthetic, such as Lidocaine, may be applied to the skin. As shown in FIG. 66, the fluid may be obtained from the patient forearm 6602. Thus, the area of application of the indicator 100 may be the area that is prepared. Other methods of preparing the skin for sample may vary based on the method the fluid is to be obtained. For example, if a blood draw via a finger prick is to be performed, preparation may only include disinfecting the surface. For a urine sample, the patient may clean around the urethra with a cleansing wipe prior to collecting the urine sample. For a saliva sample, the patient may be directed not to eat or drink for a period of time before collecting the saliva.

At block 6504, the micro-needle collector 6100 is attached to the indicator 100. In some embodiments, the micro-needle collector 6100 is pre-attached to the indicator 100, or the micro-needle collector 6100 may be substituted with a different collector suited for the type of fluid to be collected.

At block 6506, the micro-needle collector 6100 is applied to the skin. As seen in the view 6600 of FIG. 66, the micro-needle collector 6100 is attached to the indicator 100 and it is applied to the patient forearm 6602. A sufficient pressure may need to be applied to provide for sufficient engagement of the pipettes 6102 to the skin to obtain the fluid sample. The pipettes 6102 cause the fluid, here plasma, to be directed towards the indicator 100 via a capillary action. After a sufficient period of time, the micro-needle collector 6100 is removed from the patient forearm 6602. The patient may be directed to remove the indicator 100 after a pre-set period of time, or after observing a visible indication on the indicator 100 (e.g., within the indicator window 118) that means that sufficient fluid has been collected into the indicator.

At block 6508, after a sufficient time has elapsed, the patient may capture an image of the indicator 100. The image 6700 of FIG. 67 may depict the image captured by the patient. The image 6700 includes both the identifier 6110, shown here as a barcode, and the indicating window 118. The indicating window includes an image of the first and second indicating element sections 108, 210. The indicator 100 may also include a control image of known shapes and colors in close proximity to the identifier 6110 and/or the indicating window 118. The control images may aide in interpreting the image 6700 by a remote party to compensate for different light levels and colors and camera optics of the various user devices. For example, the reference indicator 116 depicted in FIG. 1E, or another similar reference indicator, may be displayed in the image 6700. Thus even if the image 6700 is taken in different lighting conditions and with different camera optics, the data of how the control image appears within the image 6700 may be used to compensate or otherwise correct for how the first and second indicating element sections 108, 210 appear within the image 6700.

At block 6510, the image captured at block 6508 is transmitted. For example, the image 6700 may serve as the image 6416 of FIG. 64 transmitted to the system 6400. In other embodiments, the image captured at block 6508 is maintained on the user device and the user device receives information to interpret the image to generate the diagnostic test results locally on the user device.

At block 6512, the image is interpreted to generate results of the diagnostic test. In the present example, the indicator may include a first indicating element section 108 that is able to detect the presence of a protein. If the protein is present, the first indicating element section 108 may display as a specific color, otherwise, the first indicating element section 108 may not appear. The second indicating element section 210 indicates whether or not the first indicating element section 108 is suitable for use. In the image 6700, the first indicating element section 108 indicates that the protein was present in the plasma fluid and the second indicating element section 210 indicates that the reading from the first indicating element section 108 is suitable for use. The identifier 6110 is also visible and able to be interpreted by the system 6400. Thus, the system 6400 is able to combine the identifier information 6412 with information from the indicator tests 6410 to generate test results 6420 for the patient.

In some embodiments, the indicator 100 may include a plurality of different indicating element sections 108, 210. Or in other embodiments, either one or both of the first and second indicating element sections 108, 210 may be broken up into a plurality of different indicating element sections that are each positioned at different locations within the indicator window 118. The indicator mapping information 6414 may store information for the location of each of the different indicating element sections 108, 210 on each indicator 108. Thus when interpreting the image to generate the results, the interpretation may be based on a mapping between the indicator mapping information 6414 and the locations of the indicating element sections 108, 210 as depicted in the image 6700.

At block 6514, the results of the diagnostic test performed by the indicator 100 are received. Thus, the system 6400 may transmit the test results 6420 to the patient, to the patient's medical records, or the like.

FIG. 68 depicts an indicator kit, in accordance with an embodiment of the present disclosure. In particular, FIG. 68 depicts the kit 6800 that includes the indicator 100, a micro-needle collector 6100, surface preparation components 6802, instructions 6808, a suction source 6810, and disposal equipment 6812. The kit 6800 may be delivered to the patient discussed in conjunction with the method 6500 above, or the patient may obtain the kit 6800 from a medical office, a pharmacy, or the like.

While the kit 6800 is generally shown as including the indicator 100, it is to be understood that any indicator disclosed herein may be included with the kit 6800. The kit further includes the micro-needle collector 6100, although other collectors may be substituted.

The surface preparation components 6802 may include sterilization chemical and applicators 6804 and a topical anesthetic 6806, as desired for the different methods to obtain the fluids.

Instructions 6808 may be printed for use and included in the kit. In other embodiments, the kit may include a link (e.g., a QR code) to direct the patient to online instructions specific to the components of the kit 6800. In other embodiments, instructions are made available with an application on the user device. The instructions may be video instructions, picture instructions, text instructions, and the like. In some embodiments, the kit 6800 may include a suction source 6810 (e.g., plunger 924).

Disposal equipment 6812 is included to ensure safe disposal of the indicator 100 and the associated accessories (e.g., micro-needle collector 6100, suction source 6810) after the fluid is obtained and the image is captured.

The indicators 100 described above may take many different form factors in accordance with various embodiments of the present disclosure. Some embodiments may be more suitable with one type of body fluid over another. For example, a form factor having a needle may be more suitable for collecting different types of fluid as the body fluid. For example, some may be suited to collect intravenous blood as the body fluid. The various form factors may be paired with first and second indicating elements associated with the type of fluid collected. FIGS. 69-74 depict various different form factors of the indicator 100.

FIG. 69 depicts an indicator in a bulb-syringe configuration, in accordance with an embodiment of the present disclosure. In particular, FIG. 69 depicts the bulb-syringe indicator 6900 that includes a bulb 6902, a nozzle 6904, a one-way valve 6906, and an opening 6910. The bulb 6902 may comprise a transparent and elastically deformable plastic. When the bulb 6902 is squeezed, air from the inside of the bulb 6902 is expelled through the one-way valve 6906. When the bulb 6902 is released, the bulb 6902 causes a suction through the first opening 6908, which may cause the fluid to be drawn through the nozzle 6904 and into the interior space of the bulb 6902.

The bulb 6902 further includes the first and second indicating element sections 108, 210. The fluid interacts with the first and second indicating element sections 108, 210 to produce results visible through the transparent bulb 6902. As with the other indicators 100, the first indicating element section 108 indicates a measurement related to the body fluid and the second indicating element section 210 indicates that the first indicating element section 108 is suitable for use. In the bulb-syringe indicator 6900 the second indicating element section 210 is disposed above (as shown in FIG. 69) the first indicating element section 108. This arrangement may provide that enough fluid has been drawn into the bulb 6902 to render the first indicating element section 108 usable when the second indicating element section 210 properly interacts with the fluid.

FIG. 70 depicts a needle and syringe configuration, in accordance with an embodiment of the present disclosure. In particular, FIG. 70 depicts the needle and syringe indicator 7000 that includes a needle 7002, an interior chamber 7004, and a plunger 7006. As compared to the bulb-syringe indicator 6900, the needle and syringe indicator 7000 may be suitable for obtaining an intravenous blood sample from a patient. Here, the needle 7002 is inserted into the patient and the plunger 7006 draws blood into the interior chamber 7004. The blood interacts with the first and second indicating element sections 108, 210.

FIG. 71 depicts a test-tube configuration of an indicator, in accordance with an embodiment of the present disclosure. In particular, FIG. 71 depicts the test tube 7100 having the first opening 7102. The first and second indicating element sections 108, 210 may comprise a color-change indicator that is adhered or affixed to an inside surface of the test tube 7100. These indicating element sections 108, 210 are visible through the surface of the test tube 7100. Fluid is added into the test tube 7100 by way of the first opening 7102.

FIG. 72 depicts a vacuum container configuration, in accordance with an embodiment of the present disclosure. In particular, FIG. 72 depicts the vacuum container 7200 that includes the membrane 7202 that initially maintains a negative pressure inside the vial 7204. The membrane 7202 may be pierced by a needle that is configured to introduce the body fluid into the interior volume of the vial 7204. The fluid then interacts with the first and second indicating element sections 108, 210. In the embodiment depicted in FIG. 72, the first indicating element sections 108 may wrapped around a perimeter of the vial 7204. FIG. 72 depicts three first indicating element sections 108, these indicating element sections may each be configured to indicate a different measurement related to a body fluid. Although FIG. 72 depicts three first indicating element sections 108, certainly other numbers of first indicating element sections may be utilized. The second indicating element section 210 is similarly disposed on the interior surface of the vial 7204, but is disposed transverse to the first indicating element sections 7204 that extend around the circumference of the vial 7204. Thus, a single second indicating element section 210 may be utilized to determine if a plurality of first indicating element sections 108 are suitable for use.

FIG. 73 depicts a molded and encapsulated indicator, in accordance with an embodiment of the present disclosure. In particular, FIG. 73 depicts the encapsulated indicator 7300 that includes the first and second indicating element sections 108, 210 encapsulated in a wall 7302. Fluid passes through the interior surface 7306 of the housing 7308. One or more openings 7304 allow the fluid to interact with the first and second indicating element sections 108, 210.

FIG. 74 depicts a cross-sectional view another embodiment of an indicator, in accordance with an embodiment of the present disclosure. In particular, FIG. 74 depicts the indicator 7400 that includes an interior chamber 7402 having a divider 7404. The divider 7404 creates a channel 7406. The first and second indicating element sections 108, 210 are disposed in the channel 7406. Fluid from the interior chamber 7402 accesses the first and second indicating element sections 108, 210 via the opening 7408. In different embodiments, the first and second indicating element sections 108, 210 may located left and right of each other (as shown in the cross-sectional view), or one may be placed behind (e.g., going into the page as shown in FIG. 74), each other.

It is appreciated that the combinations of the various components described herein may serve to filter components from the fluid as the fluid transfers from the fluid source to the first and second indicating element sections. The filtering may remove particles, cells, proteins, and the like that would otherwise interfere with visual observation of the first and second indicating element sections. Thus, components desired to be tested via the indicating element sections are able to be passed through the indicator 100 whereas components of the fluid that may interfere with reading the measurements are filtered.

For example, in an embodiment with an indicator 100 having a micro-needle collector 6100, the pipettes 6102 may server to filter whole blood cell components of the body fluid and permit plasma to pass through to the indicating element sections. In yet another embodiment, an indicator 100 having a diffuser and/or housing having channels (e.g., 154, 194, 328, and the like) extending along the fluid passage 192 may serve to filter components of the body fluid by way of the small size of the channels. In yet another embodiment, the indicating element section comprises a medium on a paper. Body fluid saturating the paper to reach the medium that indicates the measurement related to the body fluid may have components filtered out by way of passing through the paper to reach the medium. These components may be combined to selectively filter components of the body fluid while permitting testable-components of the body fluid to interact with the first and second indicating element sections.

In accordance with aspects of the invention, plural example embodiments of indicators, and housings, diffusers, end caps and closures have been described herein in several variations. Although some features have been described in the context of particular example indicators, housings, diffusers, end caps, closures, and the like, it is intended that the features may also be applied to other elements of similar function described herein, with no or routine modifications, even though all possible permutations of such features and elements have not been individually mentioned.

Those skilled in the art will recognize that the teachings of this disclosure may be modified, extended and/or applied in a variety of ways. While the foregoing has described what are considered to be the best mode and other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications, and variations that fall within the true scope of the present teachings.

What is claimed is:

1. An apparatus comprising:
a diffuser having:
a generally longitudinal shape with a proximal end opposite a distal end;
a connection tube section forming a tubular fluid passage extending from the proximal end toward the distal end along at least a part of a distance between the proximal end and the distal end;
a first indicating element section disposed between the distal end and the connection tube section, the first indicating element section indicating a measurement related to a body fluid; and
a second indicating element section, the second indicating element section indicating that the first indicating element section is suitable for use;
a housing having a generally tubular body, the diffuser is at least partially disposed in the generally tubular body;
a micro-needle collector attachable to the distal end of the housing, the micro-needle collector having:
a plurality of pipettes to draw fluid from a source to the distal end of the housing;
a divergent shape having a first end with a first cross-sectional area and a second end with a second cross-sectional area that is larger than the first cross-sectional area; and
a plate having a plurality of holes to retain a respective one of the pipettes into a respective one of the holes, the plate disposed at the second end; and
a fluid chamber cooperatively defined by the housing and the diffuser between a portion of the distal end and the proximal end, the fluid chamber adapted to receive the fluid at the distal end, wherein:
the first indicating element section is disposed in a first section of the housing;
the first section of the housing allows visibility of the first indicating element section from the outside of the housing; and
the second indicating element section disposed within the fluid chamber.

2. The apparatus of claim 1, wherein the first indicating element section is unusable if exposed to water prior to being exposed to the body fluid and the second indicating element section displays an unusable indication when exposed to water.

3. The apparatus of claim 1, wherein the body fluid is a fluid selected from the group consisting of blood, plasma, urine, and gastric aspirates.

4. The apparatus of claim 1, the housing further comprising a surface treatment on the distal end to form a seal with a fluid transport source.

5. The apparatus of claim 1, the housing further comprising an indicator window, the first indicating element section and the second indicating element section being visible through the indicator window.

6. The apparatus of claim 1, the housing further comprising a plurality of inward-facing ribs that extend radially inward into the fluid chamber to form a fluid channel between a respective pair of the inward-facing ribs.

7. The apparatus of claim 1, wherein the fluid chamber is configured to minimize a volume of fluid to saturate one or both of the first and second indicating element sections.

8. The apparatus of claim 1, further comprising a valve disposed between the distal end and the first indicating element section, the valve configured for one-way flow from the distal end towards the proximal end.

9. The apparatus of claim 1, further comprising a filter disposed between the distal end and the first indicating element section, wherein the fluid comprises blood and the filter is configured to permit passage of a plasma component of the blood.

10. The apparatus of claim 1, wherein:
the fluid comprises blood, and
the plurality of pipettes are configured to filter whole blood cell components of the fluid and pass a plasma component of the fluid.

11. The apparatus of claim 1, wherein the first indicating element section comprises a medium that changes color when exposed to the fluid, the color being associated with the measurement related to the body fluid.

12. The apparatus of claim 11, wherein the medium is disposed on a radially outer surface of the indicating element section.

13. The apparatus of claim 1, wherein the source comprises a skin surface and the micro-needle collector is configured to make punctures in the skin surface to draw the fluid from the source.

14. A method comprising:
preparing a skin surface for a sample;
attaching a micro-needle collector to an indicator;
applying the micro-needle collector to the skin surface;
capturing an image of the indicator, the indicator having:
a housing having a generally tubular body; and
a diffuser having:
a generally longitudinal shape with a proximal end opposite a distal end;
a tip section at the distal end;
a connection tube section forming a tubular fluid passage extending from the proximal end toward the distal end along at least a part of a distance between the proximal end and the distal end;
a first indicating element section disposed in a first section between the tip section and the connection tube section, the first indicating element section indicating a measurement related to a body fluid; and
a second indicating element section, the second indicating element section indicating that the first indicating element section is suitable for use; wherein:
the housing and the diffuser cooperatively define a fluid chamber between a portion of the distal end and the proximal end;
the fluid chamber is adapted to receive fluid at the distal end from the micro-needle collector;
the first section of the housing allows visibility of the first indicating element section from the outside of the housing; and
the second indicating element section is disposed within the fluid chamber;
transmitting the image; and
receiving test results, the test results indicative of the first indicating element section and the second indicating element section.

15. The method of claim 14, further comprising interpreting the image to generate the test results.

16. The method of claim 15, wherein interpreting the image to generate the test results comprises determining a location of the first indicating element section and mapping the location with indicator mapping information.

17. The method of claim 15, wherein interpreting the image to generate the test results comprises compensating for light levels, colors, and/or camera optics based on an appearance within the captured image of a control image proximate the first indicating element section, the control image having known shapes and colors.

18. The method of claim 15, wherein:
the first section of the housing further allows visibility of the second indicating element section from the outside of the housing,
capturing the image of the indicator comprises capturing an image of the first indicating element section and the second indicating element section, and
interpreting the image to generate the test results comprises interpreting the image of the first indicating element section and the second indicating element section.

19. The method of claim 15, wherein:
the first indicating element section comprises a medium that changes color when exposed to the fluid received from the micro-needle collector, the color being associated with the measurement related to the body fluid, and
interpreting the image to generate the test results comprises interpreting a color of the medium of the first indicating element section.

20. The method of claim 14, further comprising obtaining a fluid sample by the micro-needle collector through a plurality of pipettes causing the fluid sample to be directed toward the indicator.

21. The method of claim 20, wherein the fluid comprises blood, and obtaining the fluid sample further comprises the plurality of pipettes filtering whole blood cell components of the fluid and passing plasma through to the indicator.

22. The method of claim 20, wherein causing the fluid sample to be directed toward the indicator comprises capillary action.

23. The method of claim 20, wherein causing the fluid sample to be directed toward the indicator comprises suction.

24. The method of claim 14, wherein the applying the micro-needle collector to the skin surface comprises:
making a plurality of microscopic punctures in the skin surface, and
obtaining a body fluid sample by drawing the fluid from the plurality of microscopic punctures.

25. The method of claim 24, wherein drawing the fluid comprises capillary action.

26. The method of claim 24, wherein drawing the fluid comprises suction.
